(19) United States
(12) Patent Application Publication
(10) Pub. No.: US 2026/0199294 A1
(43) Pub. Date: Jul. 16, 2026

(54) PEPTIDE DRUG CONJUGATES

(71) Applicant: Oncopeptides Innovation AB, Stockholm (SE)

(72) Inventors: Kristin HAMMER, Stockholm (SE); Peter TEODOROVIC, Stockholm (SE); Rune RINGOM, Uppsala (SE)

(73) Assignee: Oncopeptides Innovation AB, Stockholm (SE)

(21) Appl. No.: 19/139,305

(22) PCT Filed: Dec. 15, 2023
(86) PCT No.: PCT/EP2023/086195

§ 371 Date: Jun. 13, 2025

(30) Foreign Application Priority Data
Dec. 16, 2022 (GB) 2219097.9

(51) Int. Cl. A61K 31/4184; A61K 45/06; A61K 47/12; A61K 47/40; A61P 35/00

(52) U.S. Cl. CPC A61K 31/4184; A61K 45/06; A61K 47/12; A61K 47/40; A61P 35/00

(57) ABSTRACT

The invention provides a pharmaceutical preparation comprising a negatively charged cyclodextrin or a pharmaceutically acceptable derivative of a negatively charged cyclodextrin, and a compound according to formula (I) as specified in the application, and the use of such preparations in the treatment of diseases such as cancer. The invention also provides related compositions, kits, methods of treatment and methods of manufacture. The invention finds particular use in the treatment of haematological cancers.

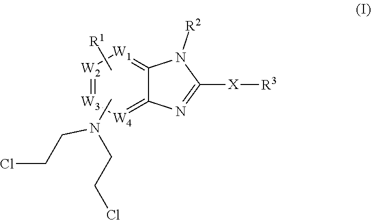

### FIELD OF THE INVENTION

The invention relates to pharmaceutical preparations comprising a negatively charged cyclodextrin or derivatives thereof and compounds that are peptide drug conjugates (PDCs), and the use of such preparations in the treatment of diseases such as cancer.

### BACKGROUND OF THE INVENTION

It has been found that compounds of formula (I) as defined below are potent anticancer agents. In particular, compounds of formula (I) display excellent in vitro cytotoxicity towards various haematological cancer cell lines. Compounds of formula (I) are also effective at reducing tumor growth in an in ovo chicken embryo xenograft model of lymphoma. Furthermore, compounds of formula (I) are readily hydrolysed within cancer cells to form metabolites that are preferentially sequestered and retained within cells and have strong alkylating activity.

There remains a need to provide a stable pharmaceutical preparation comprising the compounds of formula (I), as well as pharmaceutically acceptable salts, esters, amides or carbamates thereof, and the salts of such an ester, amide or carbamate.

### SUMMARY OF THE INVENTION

The invention provides a pharmaceutical preparation comprising a negatively charged cyclodextrin or a pharmaceutically acceptable derivative of a negatively charged cyclodextrin, and a compound according to formula (I), or a pharmaceutically acceptable salt, ester, amide or carbamate thereof, or a salt of such an ester, amide or carbamate.

The invention further provides a composition comprising a pharmaceutical preparation according to the invention and a physiologically acceptable aqueous solvent or diluent.

The invention further provides a kit comprising a pharmaceutical preparation according to the invention and a physiologically acceptable aqueous solvent or diluent.

The invention further provides a pharmaceutical preparation, composition, or kit according to the invention for use as a medicament.

The invention further provides a pharmaceutical preparation, composition, or kit according to the invention for use in the treatment or prophylaxis of cancer.

The invention further provides a method for treating a patient which comprises administering a pharmaceutically effective amount of a pharmaceutical preparation or composition according to the invention.

The invention further provides a method for the treatment or prophylaxis of cancer, comprising administering an effective amount of a pharmaceutical preparation or composition according to the invention, for example wherein said cancer is any one of hematologic/blood cell cancers such as a leukemia (for example, acute lymphoblastic leukemia including adult and childhood acute lymphoblastic leukemia; acute myeloid leukemia including adult and childhood acute myeloid leukemia; chronic lymphocytic leukemia such as B Cell chronic lymphocytic leukemia; chronic myelogenous leukemia; and hairy cell leukemia); lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; primary mediastinal large B cell lymphoma; mantle cell lymphoma; diffuse large B cell lymphoma; and primary central nervous system lymphoma); and other hematologic cancers (for example, chronic myeloproliferative disorders; multiple myeloma/plasma cell neoplasm; myelodysplastic syndromes; and myelodysplastic/myeloproliferative disorders); osteosarcoma, ovarian cancer, breast cancer; lung cancer; glioblastoma; retinoblastoma and metastases of the aforementioned cancers.

The invention further provides a composition as described hereinabove, for example a pharmaceutical composition, or a kit as described hereinabove, for use in the treatment and/or prophylaxis of a haematological cancer, for example haematological cancers and related clonal disorders (such as MGUS or amyloidosis). Haematological cancers may include for example plasma cell neoplasms and myelomas (for example MGUS, plasmocytoma, smouldering myeloma, multiple myeloma, relapsed/refractory multiple myeloma, light chain myeloma, or non-secretory myeloma and plasma cell leukemia), B-cell leukaemias (for example, acute lymphoblastic leukemia including adult and childhood acute lymphoblastic leukemia; chronic lymphocytic leukemia; and hairy cell leukemia), and B-cell derived lymphoid malignancies (for example AIDS-related lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; Waldenstrom macroglobulinemia; primary mediastinal large B cell lymphoma; diffuse large B cell lymphoma; follicular lymphoma; mantle cell lymphoma; hairy cell lymphoma; and primary central nervous system lymphoma).

Haematological cancers may include for example plasma cell neoplasms and myelomas (for example multiple myeloma or relapsed/refractory multiple myeloma), B-cell leukaemias (for example, chronic lymphocytic leukaemia (CLL), acute lymphoblastic leukaemia or hairy cell leukaemia), and B-cell derived lymphoid malignancies (for example diffuse large B cell lymphoma; follicular lymphoma; mantle cell lymphoma; hairy cell lymphoma; and primary central nervous system lymphoma). Haematological cancers may for example be selected from the group consisting of a plasma cell neoplasm or myeloma, a B-cell leukaemia, or a B-cell derived lymphoid malignancy, for example multiple myeloma, chronic lymphocytic leukaemia (CLL), or diffuse large B-cell lymphoma. In an embodiment of the invention, the haematological cancer is multiple myeloma (for example relapsed/refractory multiple myeloma) or diffuse large B-cell lymphoma, for example multiple myeloma or for example diffuse large B-cell lymphoma.

The invention further provides the use of a pharmaceutical preparation, composition, or kit according to the invention for the manufacture of a medicament for the treatment or prophylaxis of cancer, for example wherein said cancer is any one of hematologic/blood cell cancers such as a leukemia (for example, acute lymphoblastic leukemia including adult and childhood acute lymphoblastic leukemia; acute myeloid leukemia including adult and childhood acute myeloid leukemia; chronic lymphocytic leukemia such as B Cell chronic lymphocytic leukemia; chronic myelogenous leukemia; and hairy cell leukemia); lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; primary mediastinal large B cell lymphoma; mantle cell lymphoma; diffuse large B cell lymphoma; and primary central nervous system lymphoma); and other hematologic cancers (for example, chronic myeloproliferative disorders; multiple myeloma/plasma cell neoplasm; myelodysplastic syndromes; and myelodysplastic/myeloproliferative disorders); osteosarcoma, ovarian cancer, breast cancer; lung cancer; glioblastoma; retinoblastoma and metastases of the aforementioned cancers.

### BRIEF DESCRIPTION OF THE DRAWINGS

FIG. 1 shows the results of intracellular and extracellular concentration measurements of the metabolite compound (Example compound 18) (identified after treatment with Example compound 1 described herein) (FIG. 1A), and the bendamustine (FIG. 1A) and melphalan (FIG. 1B) comparators following in vitro treatment of MM.1S cells.

FIG. 2 shows the effect of Example compound 1 (left panel) and Bendamustine (right panel) on tumour growth on xenografts initiated from SU-DHL-4 human lymphoma cell line in in ovo chicken embryo model.

FIGS. 3(A) to (G) show the results of intracellular and extracellular concentration measurements of compounds described herein and their metabolites following in vitro treatment of MM.1S cells.

FIGS. 4 (A) to (C) show the results of an assay assessing DNA damage caused by compounds described herein and controls.

FIGS. 5 (A) and (B) show the results of an assay assessing DNA fragmentation caused by compounds described herein and controls. In FIG. 5B there are shown representative FACS histograms of Alexa fluor 488 intensity after treatment with a compound of Formula (I) (upper panel), Bendamustine (middle panel) or control (bottom panel).

### DETAILED DESCRIPTION OF THE INVENTION

The invention provides a pharmaceutical preparation comprising a negatively charged cyclodextrin or a pharmaceutically acceptable derivative of a negatively charged cyclodextrin, and a compound according to formula (I), or a pharmaceutically acceptable salt, ester, amide or carbamate thereof, or a salt of such an ester, amide or carbamate.

The present inventors have found that pharmaceutical preparations comprising a negatively charged cyclodextrin or a pharmaceutically acceptable derivative of a negatively charged cyclodextrin, and a compound according to formula (I), or a pharmaceutically acceptable salt, ester, amide or carbamate thereof, or a salt of such an ester, amide or carbamate are surprisingly stable.

### Compounds of Formula (I)

The compounds of formula (I) are new class of PDC that are highly cytotoxic towards human cancer cells, and particularly human haematological cancer cells. These have been disclosed in detail in International Patent Application No. PCT/EP2022/066756, which was published under the number WO2022/263679.

The compounds described as preferred in International Patent Application No. PCT/EP2022/066756 are also preferred compounds for use in the presently claimed preparations, compositions, kits, methods of treatments and uses. Preferably, the compound of formula (I) or an ester, amide or carbamate thereof, is present as a pharmaceutically acceptable salt thereof. Most preferably, the compound of formula (I) or an ester, amide or carbamate thereof, is present as a hydrochloride salt.

### PCT/EP2022/066756

It has been found a new class of PDC that are highly cytotoxic towards human cancer cells, and particularly human haematological cancer cells. As described in the Examples section (including cross-reference to PCT application number PCT/EP2022/066756 which was published under the number WO2022/263679), various example compounds described herein have been synthesised and their cytotoxicity towards several haematological cancer cell lines was tested in an in vitro cytotoxicity assay. It has been found that the compounds described herein were highly potent and display selectivity towards haematological cancer cells, as shown by the lower cytotoxicity of the compounds towards the fibroblast cell line, BJ. It has been found that Example compound 1 is particularly effective at inhibiting tumor growth in an in ovo chicken embryo xenograft model using the human lymphoma cell line, SU-DHL-4.

In a further study, it has been found that example compounds described herein were readily hydrolysed within MM.1S cancer cells to form metabolites that were sequestered and retained within the cells and had strong alkylating activity, thus demonstrating that the compounds described herein are an effective new class of PDC for the treatment or prophylaxis of cancer, and particularly the treatment or prophylaxis of haematological cancers.

Described herein are compounds of formula (I):

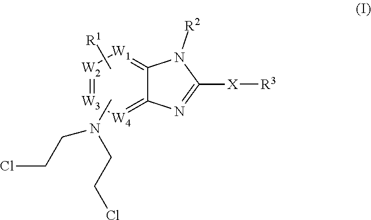

W₁, W₂, W₃ and W₄ are each CH, or one of W₁, W₂, W₃ and W₄ is N and the others are CH.

In an embodiment, W₂, W₃ and W₄ are each CH, and W₁ is N.

It is readily seen that the —N(CH₂CH₂Cl)₂ group is attached to the core part of the molecule through a carbon atom on the ring. For the relevant W group, the H in the CH group is correspondingly absent.

In preferred embodiments, W₁, W₂, W₃ and W₄ are each CH and so formula (I) has the formula (Ia):

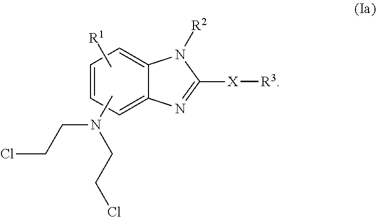

In preferred embodiments, formula (I) is according to formula (Ib),

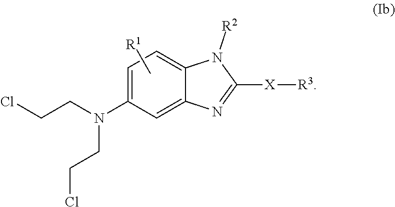

In the compounds of formula (I), X is C₁₋₆alkylene. For example, X may be C₁₋₄alkylene, C₁₋₃alkylene, C₁₋₂alkylene, C₂alkylene or C₁alkylene. X may be a linear or branched alkylene. The alkylene at position X forms a link between the imidazole moiety and the peptide portion of the compounds described herein (i.e. the R³ portion of the compound, as described below). Alkylene linkers at the X position that are one carbon or two carbons in length have been found to be particularly effective. As such, in preferred embodiments, X is a C₁alkylene (i.e. —CH₂—) or linear C₂alkylene (i.e. —CH₂—CH₂—).

In the compounds of formula (I), R¹ is selected from the group consisting of H; C₁₋₄alkyl, optionally substituted by 1, 2 or 3 groups independently selected from halogen; and halogen (for example selected from the group consisting of H; C₁₋₄alkyl; and halogen.). For example, R¹ may be selected from the group consisting of H, methyl, ethyl, propyl, isopropyl, butyl, isobutyl, sec-butyl, tert-butyl, F, Cl, Br and I. Optionally, those groups may be substituted by 1, 2 or 3 groups independently selected from halogen. In certain embodiments, R¹ is selected from the group consisting of H, methyl, ethyl, propyl, isopropyl, butyl, isobutyl, sec-butyl, tert-butyl, F and Cl. In certain preferred embodiments, R¹ is H.

In the compounds of formula (I), R² is selected from the group consisting of H; phenyl optionally substituted by 1, 2 or 3 groups independently selected from halogen; and —C₁₋₆alkyl optionally substituted by 1, 2 or 3 groups independently selected from halogen. For example, R² may be selected from the group consisting of H; phenyl optionally substituted by 1, 2 or 3 F or Cl; and —C₁₋₄alkyl (for example, methyl, ethyl, propyl, isopropyl, butyl, isobutyl, sec-butyl or tert-butyl) optionally substituted by 1, 2 or 3 F or Cl. In an embodiment, R² is selected from the group consisting of phenyl optionally substituted by 1, 2 or 3 groups independently selected from halogen; and —C₁₋₆alkyl optionally substituted by 1, 2 or 3 groups independently selected from halogen, for example —C₁₋₄alkyl (for example methyl) optionally substituted by 1, 2 or 3 groups selected from halogen. In certain embodiments, R² is an unsubstituted —C₁₋₂alkyl (for example, methyl) or an unsubstituted phenyl.

In the compounds of formula (I), R³ is a group according to formula (II):

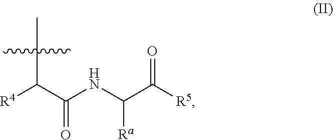

or formula (III):

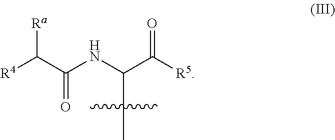

In preferred embodiments, R³ is a group according to formula (IIa):

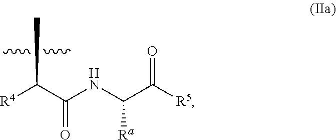

or formula (IIIa):

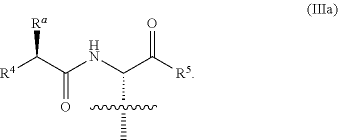

For the avoidance of doubt, [symbol] in formulas (II), (III), (IIa) and (IIIa) denotes the point of attachment of formula (II), (III), (IIa) or (IIIa) to formula (I), (Ia) or (IIa). In formulas (II), (IIa), (III) and (IIIa), R⁴ is selected from the group consisting of N(R^{c})(R^{d}) and formula (IV):

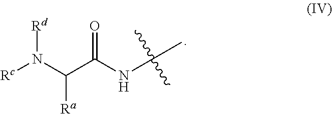

In preferred embodiments, R⁴ is selected from the group consisting of N(R^{c})(R^{d}) and formula (IVa):

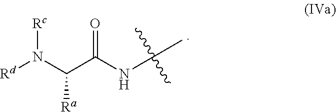

For the avoidance of doubt, [symbol] in formula (IV) and (IVa) denotes the point of attachment of formula (IV) or (IVa) to formula (II), (III), (IIa) or (IIIa).

In compounds of formula (I), when R⁴ is formula (IV) or (IVa), R⁵ is R^{b}.

R^{b} is selected from the group consisting of —OH; —N(R^{e})(R^{f}); and —OC₁₋₆alkyl optionally substituted by one or more groups selected from halogen, —OH, —CN, —N(R^{e})(R^{f}), —C₆₋₁₀aryl, or a 3 to 12 membered heterocycle comprising one or more O, N or S atoms and optionally substituted by 1, 2 or 3 halogens, and/or wherein said alkyl is optionally interrupted by 1, 2 or 3 O, N or S atoms.

For example, when R⁴ is formula (IV) or (IVa), R⁵ (which is R^{b}) may be selected from the group consisting of —OH and —OC₁₋₄alkyl (for example, methoxy, ethoxy, propoxy, isopropoxy, butoxy, isobutoxy, sec-butoxy, or tert-butoxy). Preferably, when R⁴ is formula (IV) or (IVa), R⁵ is selected from the group consisting of —OH, and —OC₁₋₃alkyl (for example, methoxy, ethoxy, propoxy, or isopropoxy). More preferably, when R⁴ is formula (IV) or (IVa), R⁵ is selected from the group consisting of methoxy, ethoxy or isopropoxy.

In compounds of formula (Ia), when R⁴ is N(R^{c})(R^{d}), R⁵ is selected from the group consisting of R^{b} and formula (V):

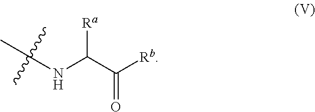

In preferred embodiments, when R⁴ is N(R^{c})(R^{d}), R⁵ is —OH, —OC₁₋₆alkyl or formula (Va):

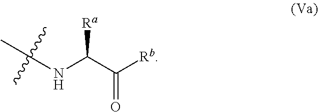

For the avoidance of doubt, [symbol] in formula (V) and (Va) denotes the point of attachment of formula (V) or (Va) to formula (II), (III), (IIa) or (IIIa).

In embodiments wherein R⁴ is N(R^{c})(R^{d}), R⁵ (being R^{b}) may be selected from the group consisting of —OH, —OC₁₋₄alkyl (for example, methoxy, ethoxy, propoxy, isopropoxy, butoxy, isobutoxy, sec-butoxy or tert-butoxy), or R⁵ may be selected from formula (V) and formula (Va). Preferably, when R⁴ is N(R^{c})(R^{d}), R⁵ is selected from the group consisting of —OH, —OC₁₋₃alkyl (for example, methoxy, ethoxy, propoxy or isopropoxy) and formula (Va). More preferably, when R⁴ is N(R^{c})(R^{d}), R⁵ is selected from the group consisting of methoxy, ethoxy or isopropoxy.

It has been found that compounds of formula (I), in which R⁴ is N(R^{c})(R^{d}), formula (IV) or (IVa), and R⁵ (being R^{b}) is —OC₁₋₃alkyl (for example, methoxy, ethoxy or isopropoxy), are especially cytotoxic towards cancer cells. Thus, in certain preferred embodiments, in the compounds of formula (I), R⁵ is —OC₁₋₃alkyl (for example, methoxy, ethoxy or isopropoxy). That is to say that, when R⁴ is N(R^{c})(R^{d}), formula (IV) or (IVa), R⁵ is preferably —OC₁₋₃alkyl (for example, methoxy, ethoxy or isopropoxy).

In certain embodiments, R⁵ is —OH.

In compounds of formula (I), each R¹ is independently selected from the group consisting of H; C₁₋₆alkyl; —CH₂-phenyl; or —CH₂-3 to 12-membered heterocyclyl comprising 1, 2, 3 or 4 heteroatoms selected from N, O and S; wherein said C₁₋₆alkyl is optionally substituted by 1, 2 or 3 substituents independently selected from the group consisting of-OH, —OC₁₋₆alkyl, —NH₂, —NHC(═NH)NH₂, —C(O)OH, —C(O)NH₂, —SH, —SCH₃, and halogen; and said phenyl or heterocyclyl is optionally substituted by 1, 2 or 3 substituents independently selected from the group consisting of halogen, —NH₂, —OH, —OC₁₋₆alkyl and —NO₂. In certain embodiments, each R^{a} is independently selected from the group consisting of H; —C₁₋₆alkyl; —CH₂-indolyl; —CH₂-phenyl; and —CH₂-5-membered heteroaryl comprising 1, 2, 3 or 4 N or S atoms; wherein C₁₋₆alkyl is optionally substituted by —OH; —NH₂; —NHC(═NH)NH₂; —C(O)OH; —C(O)NH₂; —SH; —SCH₃; or halogen (for example, F, Cl, Br or I); and phenyl is optionally substituted by 1, 2 or 3 substituents independently selected from the group consisting of halogen (for example, F, Cl, Br or I); —NH₂; —OH; —OC₁₋₆alkyl; and —NO₂. Preferably, each R^{a} is independently selected from the group consisting of H; C₁₋₄alkyl; and —CH₂-phenyl; wherein alkyl or phenyl is optionally substituted by 1 or 2 halogen (for example, F or Cl). For example, each R^{a} may independently be methyl, ethyl, propyl, isopropyl, butyl, isobutyl, sec-butyl, —CH₂-phenyl, —CH₂-fluorophenyl, —CH₂-chlorophenyl, —CH₂-difluorophenyl, or —CH₂-dichlorophenyl. In certain preferred embodiments, each R^{a} may independently be isopropyl, isobutyl, sec-butyl, —CH₂-phenyl, or —CH₂-fluorophenyl (i.e. 2-fluorobenzyl, 3-fluorobenzyl or 4-fluorobenzyl).

In compounds of formula (I), R^{b} is selected from the group consisting of —OH; —N(R^{e})(R^{f}); and —OC₁₋₆alkyl optionally substituted by one or more groups selected from halogen, —OH, —CN, —N(R^{e})(R^{f}), —C₆₋₁₀aryl, or a 3 to 12 membered heterocycle comprising one or more O, N or S atoms and optionally substituted by 1, 2 or 3 halogens, and/or wherein said alkyl is optionally interrupted by 1, 2 or 3 O, N or S atoms. In certain embodiments, R^{b} is —OH or —OC₁₋₆alkyl, wherein said alkyl is optionally interrupted by 1, 2 or 3 O or N atoms. In certain further embodiments, R^{b} is —OH or —OC₁₋₆alkyl, for example —OC₁₋₆alkyl. Preferably, R^{b} is —OC₁₋₄alkyl. For example, R^{b} may be methoxy, ethoxy, propoxy, isopropoxy, butoxy, isobutoxy or sec-butoxy. In certain preferred embodiments, R^{b} is methoxy, ethoxy or isopropoxy.

More preferably, R^{b} is ethoxy.

In certain embodiments, R^{b} is —OH.

R^{c} and R^{d} are each independently selected from the group consisting of H, —C₁₋₆alkyl, —C(O)C₁₋₆alkyl, and —CH₂-phenyl, wherein said alkyl or said phenyl is optionally substituted by 1, 2 or 3 groups selected from halogen. In preferred compounds, R^{c} is H and R^{d} is selected from H, —C₁₋₆alkyl and —C(O)C₁₋₆alkyl, wherein said alkyl is optionally substituted by 1, 2 or 3 groups selected from halogen. For example, R^{c} is H and R^{d} is selected from H, —C₁₋₄alkyl and C(O)C₁₋₄alkyl. For example, R^{c} is H and R^{d} is selected from H, methyl, ethyl, propyl, isopropyl, butyl, isobutyl, sec-butyl, —C(O)methyl, —C(O)ethyl, —C(O)propyl, —C(O)isopropyl, —C(O)butyl, —C(O)isobutyl and —C(O)sec-butyl. Preferably, R^{c} is H and R^{d} is selected from H, methyl and —C(O)methyl. Preferably, R^{c} is H and R^{d} is H.

In compounds of formula (I), R^{e} and R^{f} are each independently selected from the group consisting of H and —C₁₆alkyl, wherein said alkyl is optionally substituted by 1, 2 or 3 groups selected from halogen; or R^{e} and R^{f} together with the nitrogen atom to which they are attached form a 4-, 5- or 6-membered heterocycle which is optionally substituted by 1, 2 or 3 groups selected from halogen. In preferred compounds, R^{e} and R^{f} are each independently selected from the group consisting of H, methyl, ethyl, propyl, isopropyl, butyl, isobutyl and sec-butyl. For example, R^{e} and R^{f} are each independently selected from the group consisting of H and methyl. Preferably, R^{e} and R^{f} are both H, both methyl, or one of R^{e} and R^{f} is H and the other is methyl.

In certain embodiments, the compounds described herein are according to formula (Ia), wherein,
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIa) or (IIIa);
R⁴ is selected from the group consisting of N(R^{c})(R^{d}) and formula (IVa);
when R⁴ is formula (IVa), R⁵ (being R^{b}) is —OC₁₋₄alkyl; and when R⁴ is N(R^{c})(R^{d}), R⁵ is —OC₁₋₄alkyl or formula (Va);
each R^{a} is independently selected from the group consisting of H; —C₁₆alkyl; CH₂-indolyl; CH₂-phenyl; or —CH₂-5-membered heteroaryl comprising 1, 2, 3 or 4 N or S atoms; wherein said —C₁₋₆alkyl is optionally substituted by OH; NH₂;
—NHC(═NH)NH₂; —C(O)OH; —C(O)NH₂; —SH; —SCH₃; or halogen; and said phenyl is optionally substituted by 1, 2 or 3 substituents independently selected from the group consisting of halogen; —NH₂; —OH; —O—C₁₋₆alkyl; and —NO₂;
R^{b} is —OC₁₋₆alkyl; and
R^{c} is H and R^{d} is selected from H, —C₁₋₄alkyl and —C(O)C₁₋₄alkyl; or a pharmaceutically acceptable salt, ester, amide or carbamate thereof, including a salt of such an ester, amide or carbamate.

In certain other embodiments, the compound described herein is according to formula (Ia), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is N(R^{c})(R^{d}) or formula (IVa);
R⁵ (being R^{b}) is —OC₁₋₃alkyl; and
each R^{a} is independently selected from the group consisting of H; —C₁₆alkyl; CH₂-indolyl; CH₂-phenyl; or —CH₂-5-membered heteroaryl comprising 1, 2, 3 or 4 N or S atoms; wherein C₁₋₆alkyl is optionally substituted by OH; NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —OC₁₋₆alkyl; and —NO₂;
or a pharmaceutically acceptable salt, ester, amide or carbamate thereof, including a salt of such an ester, amide or carbamate.

It has been found that compounds described herein are especially cytotoxic towards haematological cancer cells in an in vitro cytotoxicity assay and in ovo chicken embryo xenograft model of lymphoma, when the compound is according to formula (Ia), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is N(R^{c})(R^{d}) or formula (IVa);
R⁵ (being R^{b}) is —OC₁₋₃alkyl; and
each R^{a} is independently selected from the group consisting of isopropyl, isobutyl and —CH₂-phenyl optionally substituted by 1 F (for example, 2-fluorobenzyl, 3fluorobenzyl or 4-fluorobenzyl);
or a pharmaceutically acceptable salt, ester, amide or carbamate thereof, including a salt of such an ester, amide or carbamate.

In certain other embodiments, the compounds described herein are according to formula (Ia), wherein,
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIa);
R⁴ is NH₂;
R⁵ (being R^{b}) is —OC₁₋₄alkyl; and
each R^{a} is independently selected from the group consisting of H; —C₁₆alkyl; CH₂-indolyl; CH₂-phenyl; or —CH₂-5-membered heteroaryl comprising 1, 2, 3 or 4 N or S atoms; wherein C₁₋₆alkyl is optionally substituted by OH; NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂;
or a pharmaceutically acceptable salt, ester, amide or carbamate thereof, including a salt of such an ester, amide or carbamate.

In certain other embodiments, the compounds described herein are according to formula (Ia), wherein,
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIIa);
R⁴ is NH₂;
R⁵ is —OC₁₋₄alkyl; and
each R^{a} is independently selected from the group consisting of H; —C₁₆alkyl; CH₂-indolyl; CH₂-phenyl; or —CH₂-5-membered heteroaryl comprising 1, 2, 3 or 4 N or S atoms; wherein C₁₋₆alkyl is optionally substituted by OH; NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂;
or a pharmaceutically acceptable salt, ester, amide or carbamate thereof, including a salt of such an ester, amide or carbamate.

In certain other embodiments, the compounds described herein are according to formula (Ia), wherein
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIIa);
R⁴ is formula (IVa);
R⁵ is —OC₁₋₄alkyl; and
each R^{a} is independently selected from the group consisting of H; —C₁₆alkyl; CH₂-indolyl; CH₂-phenyl; or —CH₂-5-membered heteroaryl comprising 1, 2, 3 or 4 N or S atoms; wherein —C₁₋₆alkyl is optionally substituted by OH; NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂;
or a pharmaceutically acceptable salt, ester, amide or carbamate thereof, including a salt of such an ester, amide or carbamate.

In certain preferred embodiments, the compound is:
Ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1);
Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate (Example compound 2);
Ethyl (2S)-2-[[(2S)-2-amino-4-[5-[bis(2-chloroethyl)amino]-1-methyl-benzimidazol-2-yl]butanoyl]amino]-3-methyl-butanoate (Example compound 3);
Ethyl (2S)-2-[[(2S)-2-amino-3-[5-[bis(2-chloroethyl)amino]-1-methyl-benzimidazol-2-yl]propanoyl]amino]-4-methyl-pentanoate (Example compound 4);
Ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate (Example compound 5);
Ethyl (2S)-2-[[(2S)-2-amino-3-[5-[bis(2-chloroethyl)amino]-1-methyl-benzimidazol-2-yl]propanoyl]amino]-3-methyl-butanoate (Example compound 6);
Ethyl (2S)-2-[[(2S)-2-amino-4-methyl-pentanoyl]amino]-4-[5-[bis(2-chloroethyl)amino]-1-methyl-benzimidazol-2-yl]butanoate (Example compound 7);
Ethyl (2S)-2-[[(2S)-2-amino-3-(4-fluorophenyl)propanoyl]amino]-4-[5-[bis(2-chloroethyl)amino]-1-methyl-benzimidazol-2-yl]butanoate (Example compound 8);
Ethyl (2S)-2-[[(2S)-2-amino-3-methyl-butanoyl]amino]-4-[5-[bis(2-chloroethyl)amino]-1-methyl-benzimidazol-2-yl]butanoate (Example compound 9);
Ethyl (2S)-2-[[(2S)-2-amino-4-methyl-pentanoyl]amino]-3-[5-[bis(2-chloroethyl)amino]-1-methyl-benzimidazol-2-yl]propanoate (Example compound 10);
Ethyl (2S)-2-[[(2S)-2-amino-3-(4-fluorophenyl)propanoyl]amino]-3-[5-[bis(2-chloroethyl)amino]-1-methyl-benzimidazol-2-yl]propanoate (Example compound 11);
Ethyl (2S)-2-[[(2S)-2-amino-3-methyl-butanoyl]amino]-3-[5-[bis(2-chloroethyl)amino]-1-methyl-benzimidazol-2-yl]propanoate (Example compound 12);
Ethyl (2S)-2-[[(2S)-2-amino-4-[5-[bis(2-chloroethyl)amino]-1-phenyl-benzimidazol-2-yl]butanoyl]amino]-4-methyl-pentanoate (Example compound 13);
Methyl (2S)-2-[[(2S)-2-amino-3-[5-[bis(2-chloroethyl)amino]-1-methyl-benzimidazol-2-yl]propanoyl]amino]-4-methyl-pentanoate (Example compound 14);
Isopropyl (2S)-2-[[(2S)-2-amino-3-[5-[bis(2-chloroethyl)amino]-1-methyl-benzimidazol-2-yl]propanoyl]amino]-4-methyl-pentanoate (Example compound 15);
Ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 16);
Ethyl (2S)-2-[[(2R)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 21)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-3-(4-fluorophenyl)propanoate (Example compound 22)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-4-methyl-pentanoate (Example compound 23)
2-Morpholinoethyl (2S)-2-[[(2S)-2-amino-4-[5-[bis(2-chloroethyl)amino]-1-methyl-benzimidazol-2-yl]butanoyl]amino]-4-methyl-pentanoate (Example compound 24)
2-Isopropoxyethyl (2S)-2-[[(2S)-2-amino-4-[5-[bis(2-chloroethyl)amino]-1-methyl-benzimidazol-2-yl]butanoyl]amino]-4-methyl-pentanoate (Example compound 25)
Isopropyl (2S)-2-[[(2S)-2-amino-4-[5-[bis(2-chloroethyl)amino]-1-methyl-benzimidazol-2-yl]butanoyl]amino]-4-methyl-pentanoate (Example compound 26)
Methyl (2S)-2-[[(2S)-2-amino-4-[5-[bis(2-chloroethyl)amino]-1-methyl-benzimidazol-2-yl]butanoyl]amino]-4-methyl-pentanoate (Example compound 27)
3-(Dimethylamino)propyl (2S)-2-[[(2S)-2-amino-4-[5-[bis(2-chloroethyl)amino]-1-methyl-benzimidazol-2-yl]butanoyl]amino]-4-methyl-pentanoate (Example compound 28)
(2-Methoxy-1-methyl-ethyl) (2S)-2-[[(2S)-2-amino-4-[5-[bis(2-chloroethyl)amino]-1-methyl-benzimidazol-2-yl]butanoyl]amino]-4-methyl-pentanoate (Example compound 29)
(2S)-2-[[(2S)-2-Amino-4-[5-[bis(2-chloroethyl)amino]-1-methyl-benzimidazol-2-yl]butanoyl]amino]-N,N,4-trimethyl-pentanamide (Example compound 30)
(2S)-2-[[(2S)-2-amino-4-[5-[bis(2-chloroethyl)amino]-1-methyl-benzimidazol-2-yl]butanoyl]amino]-N,4-dimethyl-pentanamide (Example compound 31)
Ethyl (2S)-2-[[(2S)-4-[5-[bis(2-chloroethyl)amino]-1-methyl-benzimidazol-2-yl]-2-(methylamino)butanoyl]amino]-4-methyl-pentanoate (Example compound 32)
Ethyl (2S)-2-[[(2S)-2-acetamido-4-[5-[bis(2-chloroethyl)amino]-1-methyl-benzimidazol-2-yl]butanoyl]amino]-3-(4-fluorophenyl)propanoate (Example 15 compound 34)
Ethyl (2S)-2-[[(2S)-2-acetamido-3-[5-[bis(2-chloroethyl)amino]-1-methyl-benzimidazol-2-yl]propanoyl]amino]-4-methyl-pentanoate (Example compound 35)
Ethyl (2S)-2-[[(2S)-2-acetamido-3-[5-[bis(2-chloroethyl)amino]-1-methyl-benzimidazol-2-yl]propanoyl]amino]-3-(4-fluorophenyl)propanoate (Example compound 36)
Ethyl (2S)-2-amino-4-[5-[bis(2-chloroethyl)amino]-1-methyl-benzimidazol-2-yl]butanoate (Example compound 38)
Ethyl (2R)-2-[[(2S)-2-amino-4-[5-[bis(2-chloroethyl)amino]-1-methyl-benzimidazol-2-yl]butanoyl]amino]-4-methyl-pentanoate (Example compound 45)
Ethyl (2S)-2-[[(2R)-2-amino-4-[5-[bis(2-chloroethyl)amino]-1-methyl-benzimidazol-2-yl]butanoyl]amino]-4-methyl-pentanoate (Example compound 46)
Ethyl (2R)-2-[[(2R)-2-amino-4-[5-[bis(2-chloroethyl)amino]-1-methyl-benzimidazol-2-yl]butanoyl]amino]-4-methyl-pentanoate (Example compound 47)
Ethyl (2S)-2-[[(2S)-2-amino-4-[6-[bis(2-chloroethyl)amino]-3-methyl-imidazo[4,5-b]pyridin-2-yl]butanoyl]amino]-4-methyl-pentanoate (Example compound 48); and
Ethyl (2S)-2-[[(2S)-2-amino-4-[5-[bis(2-chloro-1,1,2,2-tetradeuterio-ethyl)amino]-1-methyl-benzimidazol-2-yl]butanoyl]amino]-4-methyl-pentanoate (Example compound 50)
   or a pharmaceutically acceptable salt, amide or carbamate thereof, including a salt of such a amide or carbamate.

The compounds described herein may be prepared using methods known to those skilled in the art of organic chemistry. Exemplary procedures for the preparation of compounds of formula (I) are described in the Examples section (including cross-reference to PCT application number PCT/EP2022/066756 which was published under the number WO2022/263679).

In embodiments, the compound described herein may comprise an isotope atom. As defined herein, an isotope atom is an atom of an element that is not the most common naturally occurring isotope. Deuterium is a safe and stable isotope of hydrogen. In one embodiment, the compound described herein has a deuterium abundance level greater than the naturally occurring abundance of deuterium. The naturally occurring abundance of deuterium is 0.0156 mol %, wherein mol % is the percentage of the total moles of a sample's hydrogen that is deuterium. Therefore, in 1 mole of naturally occurring hydrogen 0.156 mmol is deuterium, or in a sample of 6.022×10²³ naturally occurring hydrogen atoms there are 9.39×10¹⁹ atoms of deuterium, or in a sample of 6413 naturally occurring hydrogen atoms there is one atom of deuterium. A deuterium abundance level greater than the naturally occurring abundance of deuterium may be at least 1 mol %, 5 mol %, 10 mol %, 50 mol %, 90 mol % or 98 mol % deuterium. In certain embodiments, the compound described herein has a deuterium abundance level of at least 1 mol %, 5 mol %, 10 mol %, 50 mol %, 90 mol % or 98 mol % deuterium. Procedures for preparing deuterated compounds are known in the art. See for example Sajiki, New Horizons of Process Chemistry (2017), Springer, pg 29-40, and Hanson, The Organic Chemistry of Isotopic Labelling (2011), Chapter 3, RSC Publishing.

Depending upon the substituents present in compounds described herein, the compounds may form esters, amides, carbamates and/or salts. Salts of compounds described herein which are suitable for use in medicine are those wherein a counterion is pharmaceutically acceptable. However, salts having non-pharmaceutically acceptable counterions may, for example, be used as intermediates in the preparation of the compounds described herein and their pharmaceutically acceptable salts, and physiologically functional derivatives. The term “physiologically functional derivative” refers to a chemical derivative of a compound described herein that has the same physiological function as the compound described herein, for example, by being convertible in the body thereto. Esters, amides and carbamates are examples of physiologically functional derivatives.

Suitable salt forms of the compounds described herein include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, or such as saturated or unsaturated dicarboxylic acids, or such as hydroxycarboxylic acids, or such as amino acids, or with organic sulfonic acids, such as (C₁-C₄)alkyl or arylsulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids such as oxalic, while not in themselves pharmaceutically acceptable, may be useful as intermediates in obtaining the compounds described herein and their pharmaceutical acceptable acid addition salts.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono, di or trilower alkylamine, for example ethyl, tertbutyl, diethyl, diisopropyl, triethyl, tributyl or dimethyl-propylamine, or a mono, di or trihydroxy-lower alkylamine, for example mono, di or triethanolamine. Corresponding internal salts may furthermore be formed.

Preferred salts of a compound described herein include acid addition salts such as those formed from hydrochloric, hydrobromic, acetic, p-toluenesulfonic, tartaric, sulphuric, succinic, phosphoric, oxalic, nitric, methanesulfonic, malic, maleic and citric acid. More preferably, the salt of a compound described herein is the hydrochloride salt (i.e. the addition salt formed from hydrochloric acid).

A compound which is itself inactive, but which, upon administration to the recipient, is capable of being converted into an active drug compound is known as a “prodrug”. A prodrug may, for example, be converted within the body, e.g. by hydrolysis in the blood, into an active form that has medical effects. Pharmaceutically acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987. Examples of prodrugs include esters, amides and carbamates.

Compounds described herein may have an appropriate group converted to an ester, an amide or a carbamate. Thus typical ester and amide groups formed from an acid group in a compound described herein include —COOR^{G}, CONR^{G}₂, SO₂OR^{G}, or SO₂N(R^{G})₂, while typical ester and amide and carbamate groups formed from an OH or —NHR^{G} group in the compound described herein include OC(O)R^{G}, NR^{G}C(O)R^{G}, NR^{G}CO₂R^{G}, OSO₂R^{G}, and —NR^{G}SO₂R^{G}, where R^{G} is selected from the group consisting of C₁₋₈alkyl, C₂₋₈alkenyl, C₂₋₈alkynyl, C₃₋₈cycloalkyl and C₃₋₈cycloalkylC₁₋₈alkyl, haloC₁₈alkyl, dihaloC₁₋₈alkyl, trihaloC₁₈alkyl, phenyl and phenylC₁₄alkyl; more preferably R^{G} is selected from the group consisting of C₁₋₆alkyl, C₂₆alkenyl, C₂₆alkynyl, C₃₋₈cycloalkyl and C₃₋₈cycloalkylC₁₋₆alkyl.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2^{nd} edn, R. Lui, CRC Press, page 553, and Byrn et al., Pharm. Res., 12(7), 1995, 945-954. Before it is made up in solution, a compound described herein, as well as esters, amides, carbamates and/or salts thereof, may be in the form of a solvate. Solvates of a compound described herein that are suitable for use as a medicament are those wherein the associated solvent is pharmaceutically acceptable. For example a hydrate is pharmaceutically acceptable solvate.

It has been found that compounds described herein in which R⁵ is selected from the group consisting of —OC₁₋₆alkyl, formula (V)

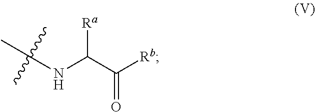

and formula (Va)

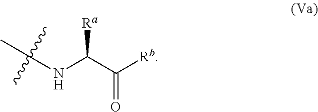

and R^{b} is a group selected from —N(R^{e})(R^{f}) and —OC₁₋₆alkyl, are readily hydrolysed within cancer cells to form metabolites that are preferentially sequestered and retained within cells and have strong alkylating activity.

Thus, also described herein is a metabolite, wherein said metabolite has a structure according to formula (I), (Ia) or (Ib), wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen;
R³ is a group according to formula (II), (III), (IIa) or (IIIa);
R⁴ is selected from the group consisting of N(R^{c})(R^{d}), formula (IV) or (IVa);
when R⁴ is formula (IV) or (IVa), R⁵ is —OH; and when R⁴ is N(R^{c})(R^{d}), R⁵ is —OH, formula (V) or (Va);
each R^{a} is independently selected from the group consisting of H; —C₁₆alkyl; CH₂-phenyl; or —CH₂-3 to 12-membered heterocyclyl comprising 1, 2, 3 or 4 heteroatoms selected from N, O and S; wherein said —C₁₋₆alkyl is optionally substituted by 1, 2 or 3 substituents independently selected from the group consisting of —OH, —OC₁₋₆alkyl, NH₂, —NHC(═NH)NH₂, —C(O)OH, —C(O)NH₂, —SH, —SCH₃, and halogen; and said phenyl or heterocyclyl is optionally substituted by 1, 2 or 3 substituents independently selected from the group consisting of halogen, —NH₂, —OH, —OC₁₋₆alkyl and —NO₂ and
R^{b} is OH;
or a salt thereof.

Preferred radicals within the metabolite compounds as described immediately above are as described hereinabove regarding compounds of Formula (I), (Ia) and (Ib). For example, in certain embodiments, the metabolite has a structure according to formula (I), (Ia) or (Ib), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is selected from the group consisting of N(R^{c})(R^{d}) or formula (IVa);
when R⁴ is formula (IVa), R⁵ is —OH; and when R⁴ is N(R^{c})(R^{d}), R⁵ is —OH or formula (Va);
each R^{a} is independently selected from the group consisting of H; —C₁₆alkyl; CH₂-indolyl; CH₂-phenyl; or —CH₂-5-membered heteroaryl comprising 1, 2, 3 or 4 N or S atoms; wherein —C₁₋₆alkyl is optionally substituted by OH; NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂; and
R^{b} is OH;
or a salt thereof.

In certain embodiments, the metabolite has a structure according to formula (I), (Ia) or (Ib), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is NH₂ or formula (IVa);
R⁵ is —OH;
each R^{a} is independently selected from the group consisting of H; —C₁₆alkyl; CH₂-indolyl; CH₂-phenyl; or —CH₂-5-membered heteroaryl comprising 1, 2, 3 or 4 N or S atoms; wherein C₁₋₆alkyl is optionally substituted by OH; NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —OC₁₋₆alkyl; and —NO₂. Preferably, each R^{a} is independently selected from the group consisting of isopropyl, isobutyl and —CH₂-phenyl optionally substituted by 1 F (for example, 2-fluorobenzyl, 3fluorobenzyl or 4-fluorobenzyl);
or a salt thereof.

In certain preferred embodiments, the metabolite is:
(2S)-2-[[(2S)-2-amino-4-[5-[bis(2-chloroethyl)amino]-1-methyl-benzimidazol-2-yl]butanoyl]amino]-3-methyl-butanoic acid (Example compound 19); and
(2S)-2-[[(2S)-2-amino-4-[5-[bis(2-chloroethyl)amino]-1-methyl-benzimidazol-2-yl]butanoyl]amino]-4-methyl-pentanoic acid (Example compound 20);
(2S)-2-[[(2S)-4-[5-[bis(2-chloroethyl)amino]-1-methyl-benzimidazol-2-yl]-2-(methylamino)butanoyl]amino]-4-methyl-pentanoic acid (Example compound 33)
(2S)-2-[[(2S)-2-Amino-4-methyl-pentanoyl]amino]-4-[5-[bis(2-chloroethyl)amino]-1-methyl-benzimidazol-2-yl]butanoic acid (Example compound 37)
(2S)-2-[[(2S)-2-Amino-4-[5-[bis(2-chloroethyl)amino]-1-methyl-benzimidazol-2-yl]butanoyl]amino]-3-(4-fluorophenyl)propanoic acid (Example compound 39)
(2S)-2-[[(2S)-2-Amino-4-[5-[bis(2-chloroethyl)amino]-1-phenyl-benzimidazol-2-yl]butanoyl]amino]-4-methyl-pentanoic acid (Example compound 40)
2S)-2-[[(2S)-2-amino-3-[5-[bis(2-chloroethyl)amino]-1-methyl-benzimidazol-2-yl]propanoyl]amino]-3-methyl-butanoic acid (Example compound 42)
(2S)-2-[[(2S)-2-amino-4-methyl-pentanoyl]amino]-3-[5-[bis(2-chloroethyl)amino]-1-methyl-benzimidazol-2-yl]propanoic acid (Example compound 43)
(2S)-2-[[(2S)-2-amino-3-[5-[bis(2-chloroethyl)amino]-1-methyl-benzimidazol-2-yl]propanoyl]amino]-4-methyl-pentanoic acid (Example compound 44); and
(2S)-2-[[(2S)-2-amino-4-[5-[bis(2-chloro-1,1,2,2-tetradeuterio-ethyl)amino]-1-methyl-benzimidazol-2-yl]butanoyl]amino]-4-methyl-pentanoic acid (Example compound 51),
   or a salt thereof.

It has been found that compounds described herein are readily further hydrolysed within cancer cells to form further, more advanced metabolites that are preferentially sequestered and retained within cells and have strong alkylating activity. Thus, also described herein is a metabolite that has a structure according to formula (I), (Ia) or (Ib), wherein,
X is C₁₋₆alkylene;
W₁, W₂, W₃ and W₄ are each CH, or one of W₁, W₂, W₃ and W₄ is N and the others are CH;
R¹ is selected from the group consisting of H; —C₁₋₄alkyl, optionally substituted by 1, 2 or 3 groups independently selected from halogen; and halogen (for example, F or Cl);
R² is selected from the group consisting of H; phenyl optionally substituted by 1, 2 or 3 groups independently selected from halogen (for example, F or Cl); and —C₁₋₆alkyl optionally substituted by 1, 2 or 3 groups independently selected from halogen (for example, F or Cl); and
R³ is formula (VIa)

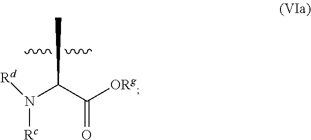

wherein, R^{c} and R^{d} are each independently selected from the group consisting of H, —C₁₋₆alkyl, —C(O)C₁₋₆alkyl and —CH₂-phenyl, wherein said alkyl or said phenyl is optionally substituted by 1, 2 or 3 groups selected from halogen (preferably, R^{c} and R^{d} are both H); and
R^{g} is selected from the group consisting of H and —C₁₋₆alkyl, wherein said alkyl is optionally substituted by 1, 2 or 3 groups selected from halogen (preferably R^{g} is H).

For the avoidance of doubt, [symbol] in formulas (VI) and (VIa) denotes the point of attachment of formula (VI) or (VIa) to formula (I), (Ia) or (Ib).

In certain embodiments, the metabolite has a structure according to formula (I), (Ia) or (Ib), wherein,
X is C₁₋₆alkylene;
R¹ is selected from the group consisting of H; —C₁₋₄alkyl; and halogen (for example, F or Cl);
R² is selected from the group consisting of H; phenyl substituted by 1, 2 or 3 halogens (for example, F or Cl); and —C₁₋₆alkyl substituted by 1, 2 or 3 halogens (for example, F or Cl) (for example selected from the group consisting of phenyl substituted by 1, 2 or 3 halogens (for example, F or Cl); and —C₁₋₆alkyl substituted by 1, 2 or 3 halogens (for example, F or Cl));
R³ is formula (VIa); and
R^{c} and R^{d} are both H;
or a salt thereof.

In certain other embodiments, the metabolite has a structure according to formula (I), (Ia) or (Ib), wherein,
X is C₁alkylene (i.e. —CH₂—) or C₂alkylene (i.e. —CH₂—CH₂—);
R¹ is selected from the group consisting of H; —C₁₋₄alkyl; and halogen (for example, F or Cl);
R² is selected from the group consisting of H; phenyl optionally substituted by 1, 2 or 3 halogens (for example, F or Cl); and —C₁₋₆alkyl optionally substituted by 1, 2 or 3 halogens (for example, F or Cl); and
R³ is formula (VIa); and
R^{c} and R^{d} are both H;
or a salt thereof.

Preferably, the compound is one of formula (Ia) or (Ib).

Thus, also described herein is a compound that has a structure according to formula (I), (Ia) or (Ib), wherein,
X is C₂₋₆alkylene;
W₁, W₂, W₃ and W₄ are each CH, or one of W₁, W₂, W₃ and W₄ is N and the others are CH;
R¹ is selected from the group consisting of H; —C₁₋₄alkyl, optionally substituted by 1, 2 or 3 groups independently selected from halogen; and halogen (for example, F or Cl);
R² is selected from the group consisting of H; phenyl optionally substituted by 1, 2 or 3 groups independently selected from halogen (for example, F or Cl); and —C₁₋₆alkyl optionally substituted by 1, 2 or 3 groups independently selected from halogen (for example, F or Cl); and
R³ is formula (VI)

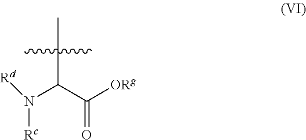

wherein, R^{c} and R^{d} are each independently selected from the group consisting of H, —C₁₋₆alkyl, —C(O)C₁₋₆alkyl and —CH₂-phenyl, wherein said alkyl or said phenyl is optionally substituted by 1, 2 or 3 groups selected from halogen (for example R^{c} and R^{d} are both H); and
R^{g} is selected from the group consisting of H and —C₁₋₆alkyl, wherein said alkyl is optionally substituted by 1, 2 or 3 groups selected from halogen (for example R^{g} is H).

### Negatively Charged Cyclodextrins

A negatively charged cyclodextrin is a cyclodextrin or a derivative of a cyclodextrin which bears one or more negative charge.

For example, the negatively charged cyclodextrin or derivative thereof bears 4 to 10 negative charges. Preferably, the negatively charged cyclodextrin or derivative thereof bears 5 to 8 negative charges.

Preferably, the cyclodextrin or derivative thereof bears one or more negative charge at a pH above 2, for example pH 2 to pH 8 or pH 3 to pH 5.5.

Preferably, the negative charge is found in a substituent of the cyclodextrin, for example in a carboxylic acid group or a sulfoalkyl group, preferably in a sulfoalkyl group, for example a sulfobutyl group.

The average number of sulfoalkyl groups (e.g. sulfobutyl groups) per cyclodextrin ring, expressed as average degree of substitution is from 4 to 10. Preferably, the average number of sulfoalkyl groups (e.g. sulfobutyl groups) per cyclodextrin ring, expressed as average degree of substitution is from 5 to 8.

The negatively charged cyclodextrin or derivative thereof can be of any size, e.g. a negatively charged α-cyclodextrin or derivative thereof, a negatively charged β-cyclodextrin or derivative thereof, or a negatively charged γ-cyclodextrin or derivative thereof. Preferably, the negatively charged cyclodextrin or derivative thereof is a negatively charged β-cyclodextrin or derivative thereof.

A preferred negatively charged cyclodextrin is a sulfoalkyl ether β-cyclodextrin wherein the number of sulfoalkyl groups per cyclodextrin ring, expressed as average degree of substitution is from 4 to 10. More preferably, negatively charged cyclodextrin is a sulfobutyl ether β-cyclodextrin wherein the average number of sulfobutyl groups per cyclodextrin ring, expressed as average degree of substitution is from 5 to 8. The structural formula of such a sulfobutyl ether β-cyclodextrin is shown below, with an exemplary value of m for the degree of substitution.

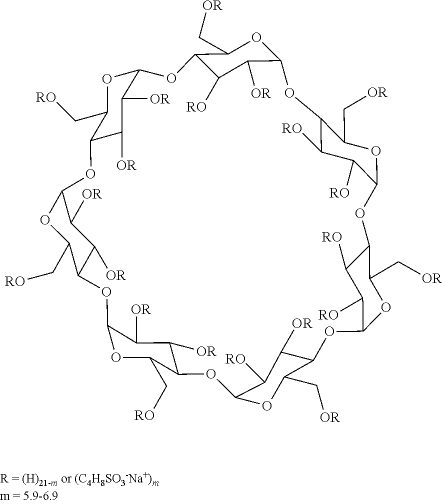

One such negatively charged cyclodextrin, which is particularly preferred is Betadex Sulfobutyl Ether Sodium (also known as “Dexolve” or “Captisol”), as described in Handbook of Pharmaceutical excipients. 8th edn. London: Pharmaceutical Press (2017) pages 112-117.

### Pharmaceutical Preparations

It has been found that preparations comprising compounds of formula (I) and negatively charged cyclodextrins as described above are particularly stable. Thus, the present invention provides pharmaceutical preparations comprising a negatively charged cyclodextrin or a pharmaceutically acceptable derivative of a negatively charged cyclodextrin, and a compound according to formula (I), or a pharmaceutically acceptable salt, ester, amide or carbamate thereof, or a salt of such an ester, amide or carbamate.

Preferably, the pharmaceutical preparation of the invention comprises a negatively charged cyclodextrin or a pharmaceutically acceptable derivative of a negatively charged cyclodextrin which bears 4 to 10 negative charges, more preferably, 5 to 8 negative charges.

Preferably, the pharmaceutical preparation of the invention comprises a negatively charged cyclodextrin or a pharmaceutically acceptable derivative of a negatively charged cyclodextrin bears one or more negative charge at a pH above 2, for example pH 2 to pH 8 or pH 3 to pH 5.5.

Preferably, the pharmaceutical preparation of the invention comprises a pharmaceutically acceptable derivative of a negatively charged cyclodextrin, for example a carboxylic acid derivatized cyclodextrin or a sulfoalkyl ether derivatized cyclodextrin. More preferably, the pharmaceutical preparation of the invention comprises a sulfoalkyl ether derivatized cyclodextrin, for example a sulfobutyl ether derivatized cyclodextrin.

When the pharmaceutical preparation of the invention comprises a sulfoalkyl ether derivatized cyclodextrin (for example a sulfobutyl ether derivatized cyclodextrin), the average number of sulfoalkyl groups (e.g. sulfobutyl groups) per cyclodextrin ring, expressed as average degree of substitution is from preferably from 4 to 10, more preferably from 5 to 8.

Preferably, the pharmaceutical preparation of the invention comprises a negatively charged α-cyclodextrin or a pharmaceutically acceptable derivative of a negatively charged α-cyclodextrin, a negatively charged β-cyclodextrin or a pharmaceutically acceptable derivative of a β-cyclodextrin, or a negatively charged γ-cyclodextrin or a pharmaceutically acceptable derivative of a γ-cyclodextrin. More preferably, the pharmaceutical preparation of the invention comprises a negatively charged β-cyclodextrin or a pharmaceutically acceptable derivative of a β-cyclodextrin.

Preferably, the pharmaceutical preparation of the invention comprises a sulfoalkyl ether β-cyclodextrin wherein the number of sulfoalkyl groups per cyclodextrin ring, expressed as average degree of substitution is from 4 to 10.

More preferably, the pharmaceutical preparation of the invention comprises a sulfobutyl ether β-cyclodextrin wherein the average number of sulfobutyl groups per cyclodextrin ring, expressed as average degree of substitution is from 5 to 8.

More preferably again, the pharmaceutical preparation of the invention comprises a sulfobutyl ether β-cyclodextrin with the structure shown below (with an exemplary value of m for the degree of substitution):

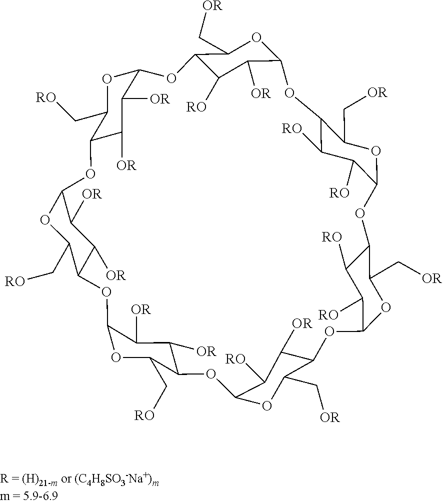

Most preferably, the pharmaceutical preparation of the invention comprises a Betadex Sulfobutyl Ether Sodium. The pharmaceutical preparation of the invention comprises a compound according to formula (I), or a pharmaceutically acceptable salt, ester, amide or carbamate thereof, or a salt of such an ester, amide or carbamate

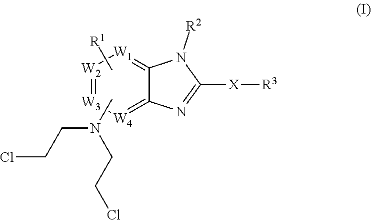

wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is a group according to formula (11):

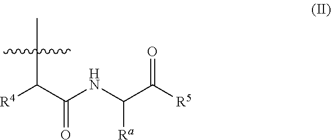

or formula (III):

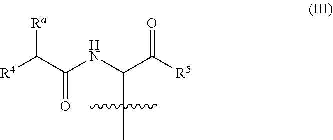

wherein,
R⁴ is selected from the group consisting of N(R^{c})(R^{d}) and formula (IV):

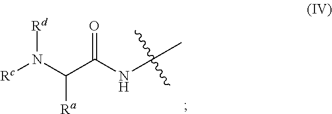

and
when R⁴ is formula (IV), R⁵ is R^{b}; and when R⁴ is N(R^{c})(R^{d}), R⁵ is selected from the group consisting of R^{b} and formula (V):

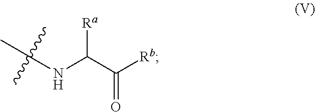

wherein each R^{a} is independently selected from the group consisting of H; C₁alkyl; CH₂-phenyl; or —CH₂-3 to 12-membered heterocyclyl comprising 1, 2, 3 or 4 heteroatoms selected from N, O and S; wherein said C₁₋₆alkyl is optionally substituted by 1, 2 or 3 substituents independently selected from the group consisting of-OH, —OC₁₋₆alkyl, NH₂, —NHC(═NH)NH₂, —C(O)OH, —C(O)NH₂, —SH, —SCH₃, and halogen; and said phenyl or heterocyclyl is optionally substituted by 1, 2 or 3 substituents independently selected from the group consisting of halogen, —NH₂, —OH, —OC₁₋₆alkyl and —NO₂;
R^{b} is selected from the group consisting of —OH; —N(R^{e})(R^{f}); and —OC₁₋₆alkyl optionally substituted by one or more groups selected from halogen, —OH, —CN, —N(R^{e})(R^{f}), —C₆₋₁₀aryl, or 3 to 12 membered heterocycle comprising one or more O, N or S atoms and optionally substituted by 1, 2 or 3 halogens, and/or wherein said alkyl is optionally interrupted by 1, 2 or 3 O, N or S atoms;
R^{c} and R^{d} are each independently selected from the group consisting of H; —C₁₆alkyl, C(O)C₁₆alkyl; and —CH₂-phenyl, wherein said alkyl or said phenyl is optionally substituted by 1, 2 or 3 groups selected from halogen; and
R^{e} and R^{f} are each independently selected from the group consisting of H and —C₁₆alkyl, wherein said alkyl is optionally substituted by 1, 2 or 3 groups selected from halogen; or R^{e} and R^{f} together with the nitrogen atom to which they are attached form a 4-, 5- or 6-membered heterocycle which is optionally substituted by 1, 2 or 3 groups selected from halogen.

Preferably, the pharmaceutical preparation of the invention comprises a compound of formula (I) or a pharmaceutically acceptable salt, ester, amide or carbamate thereof, or a salt of such an ester, amide or carbamate has the structure (Ia):

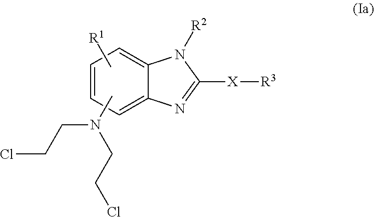

for example structure (Ib):

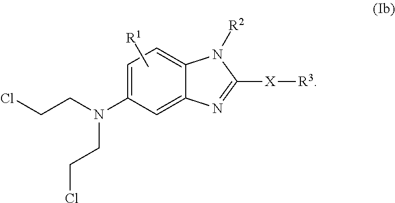

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein R¹ is H.

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein R² is selected from the group consisting of phenyl optionally substituted by 1, 2 or 3 halogens; and —C₁₋₆alkyl optionally substituted by 1, 2 or 3 halogens; for example —C₁₋₄alkyl optionally substituted by 1, 2 or 3 halogens, for example methyl.

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein X is —CH₂— or —CH₂—CH₂—.

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein R³ is a group according to formula (IIa):

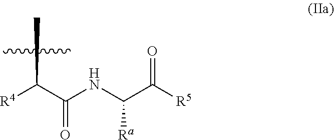

or formula (IIIa):

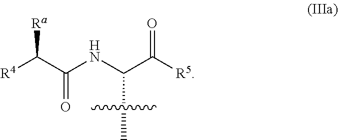

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein R⁴ is selected from the group consisting of N(R^{c})(R^{d}) and formula (IVa):

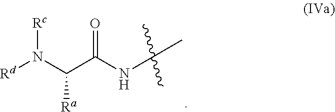

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein, when R⁴ is formula (IV) or (IVa), R⁵ is R^{b}; and when R⁴ is N(R^{c})(R^{d}), R⁵ is selected from the group consisting of R^{b} and formula (Va), wherein formula (Va) has the following

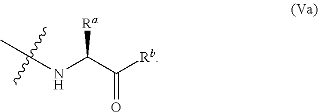

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein each R⁴ is independently selected from the group consisting of C₁alkyl and CH₂-phenyl; wherein said C₁₋₆alkyl is optionally substituted by OH; NH₂; —NHC(═NH)NH₂; —C(O)OH; —C(O)NH₂; —SH; —SCH₃; or halogen (for example, F or Cl); and said phenyl is optionally substituted by 1, 2 or 3 substituents independently selected from the group consisting of halogen (for example, F or Cl); —NH₂; —OH; —O—C₁₋₆alkyl; and —NO₂.

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein each R^{a} is selected from the group consisting of —C₁₆alkyl and CH₂-phenyl; wherein said phenyl is optionally substituted by 1, 2 or 3 halogen substituents.

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein when R⁴ is formula (IV) or (IVa), R⁵ is —OC₁₋₆alkyl; and when R⁴ is NH₂, R⁵ is —OC₁₋₆alkyl or formula (V) or (Va).

Preferably, the pharmaceutical preparation of the invention comprises a compound of formula (I), (Ia) or (Ib), or a pharmaceutically acceptable salt, ester, amide or carbamate thereof, or a salt of such an ester, amide or carbamate wherein when R^{b} is —OC₁₋₆alkyl, it is selected from the group consisting of methoxy, ethoxy and isopropoxy.

Preferably, the pharmaceutical preparation of the invention comprises a compound of formula (I), or a pharmaceutically acceptable salt, ester, amide or carbamate thereof, or a salt of such an ester, amide or carbamate selected from the group consisting of:
Ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1);
Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate (Example compound 2);
Ethyl (2S)-2-[[(2S)-2-amino-4-[5-[bis(2-chloroethyl)amino]-1-methyl-benzimidazol-2-yl]butanoyl]amino]-3-methyl-butanoate (Example compound 3);
Ethyl (2S)-2-[[(2S)-2-amino-3-[5-[bis(2-chloroethyl)amino]-1-methyl-benzimidazol-2-yl]propanoyl]amino]-4-methyl-pentanoate (Example compound 4);
Ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate (Example compound 5);
Ethyl (2S)-2-[[(2S)-2-amino-3-[5-[bis(2-chloroethyl)amino]-1-methyl-benzimidazol-2-yl]propanoyl]amino]-3-methyl-butanoate (Example compound 6);
Ethyl (2S)-2-[[(2S)-2-amino-4-methyl-pentanoyl]amino]-4-[5-[bis(2-chloroethyl)amino]-1-methyl-benzimidazol-2-yl]butanoate (Example compound 7);
Ethyl (2S)-2-[[(2S)-2-amino-3-(4-fluorophenyl)propanoyl]amino]-4-[5-[bis(2-chloroethyl)amino]-1-methyl-benzimidazol-2-yl]butanoate (Example compound 8);
Ethyl (2S)-2-[[(2S)-2-amino-3-methyl-butanoyl]amino]-4-[5-[bis(2-chloroethyl)amino]-1-methyl-benzimidazol-2-yl]butanoate (Example compound 9);
Ethyl (2S)-2-[[(2S)-2-amino-4-methyl-pentanoyl]amino]-3-[5-[bis(2-chloroethyl)amino]-1-methyl-benzimidazol-2-yl]propanoate (Example compound 10);
Ethyl (2S)-2-[[(2S)-2-amino-3-(4-fluorophenyl)propanoyl]amino]-3-[5-[bis(2-chloroethyl)amino]-1-methyl-benzimidazol-2-yl]propanoate (Example compound 11);
Ethyl (2S)-2-[[(2S)-2-amino-3-methyl-butanoyl]amino]-3-[5-[bis(2-chloroethyl)amino]-1-methyl-benzimidazol-2-yl]propanoate (Example compound 12);
Ethyl (2S)-2-[[(2S)-2-amino-4-[5-[bis(2-chloroethyl)amino]-1-phenyl-benzimidazol-2-yl]butanoyl]amino]-4-methyl-pentanoate (Example compound 13);
Methyl (2S)-2-[[(2S)-2-amino-3-[5-[bis(2-chloroethyl)amino]-1-methyl-benzimidazol-2-yl]propanoyl]amino]-4-methyl-pentanoate (Example compound 14);
Isopropyl (2S)-2-[[(2S)-2-amino-3-[5-[bis(2-chloroethyl)amino]-1-methyl-benzimidazol-2-yl]propanoyl]amino]-4-methyl-pentanoate (Example compound 15);
ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 16);
Ethyl (2S)-2-[[(2R)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 21)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-3-(4-fluorophenyl)propanoate (Example compound 22)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-4-methyl-pentanoate (Example compound 23)
2-Morpholinoethyl (2S)-2-[[(2S)-2-amino-4-[5-[bis(2-chloroethyl)amino]-1-methyl-benzimidazol-2-yl]butanoyl]amino]-4-methyl-pentanoate (Example compound 24)
2-Isopropoxyethyl (2S)-2-[[(2S)-2-amino-4-[5-[bis(2-chloroethyl)amino]-1-methyl-benzimidazol-2-yl]butanoyl]amino]-4-methyl-pentanoate (Example compound 25)
Isopropyl (2S)-2-[[(2S)-2-amino-4-[5-[bis(2-chloroethyl)amino]-1-methyl-benzimidazol-2-yl]butanoyl]amino]-4-methyl-pentanoate (Example compound 26)
Methyl (2S)-2-[[(2S)-2-amino-4-[5-[bis(2-chloroethyl)amino]-1-methyl-benzimidazol-2-yl]butanoyl]amino]-4-methyl-pentanoate (Example compound 27)
3-(Dimethylamino)propyl (2S)-2-[[(2S)-2-amino-4-[5-[bis(2-chloroethyl)amino]-1-methyl-benzimidazol-2-yl]butanoyl]amino]-4-methyl-pentanoate (Example compound 28)
(2-Methoxy-1-methyl-ethyl) (2S)-2-[[(2S)-2-amino-4-[5-[bis(2-chloroethyl)amino]-1-methyl-benzimidazol-2-yl]butanoyl]amino]-4-methyl-pentanoate (Example compound 29)
(2S)-2-[[(2S)-2-Amino-4-[5-[bis(2-chloroethyl)amino]-1-methyl-benzimidazol-2-yl]butanoyl]amino]-N,N,4-trimethyl-pentanamide (Example compound 30)
(2S)-2-[[(2S)-2-amino-4-[5-[bis(2-chloroethyl)amino]-1-methyl-benzimidazol-2-yl]butanoyl]amino]-N,4-dimethyl-pentanamide (Example compound 31)
Ethyl (2S)-2-[[(2S)-4-[5-[bis(2-chloroethyl)amino]-1-methyl-benzimidazol-2-yl]-2-(methylamino)butanoyl]amino]-4-methyl-pentanoate (Example compound 32)
Ethyl (2S)-2-[[(2S)-2-acetamido-4-[5-[bis(2-chloroethyl)amino]-1-methyl-benzimidazol-2-yl]butanoyl]amino]-3-(4-fluorophenyl)propanoate (Example compound 34)
Ethyl (2S)-2-[[(2S)-2-acetamido-3-[5-[bis(2-chloroethyl)amino]-1-methyl-benzimidazol-2-yl]propanoyl]amino]-4-methyl-pentanoate (Example compound 35)
Ethyl (2S)-2-[[(2S)-2-acetamido-3-[5-[bis(2-chloroethyl)amino]-1-methyl-benzimidazol-2-yl]propanoyl]amino]-3-(4-fluorophenyl)propanoate (Example compound 36)
Ethyl (2S)-2-amino-4-[5-[bis(2-chloroethyl)amino]-1-methyl-benzimidazol-2-yl]butanoate (Example compound 38)
Ethyl (2R)-2-[[(2S)-2-amino-4-[5-[bis(2-chloroethyl)amino]-1-methyl-benzimidazol-2-yl]butanoyl]amino]-4-methyl-pentanoate (Example compound 45)
Ethyl (2S)-2-[[(2R)-2-amino-4-[5-[bis(2-chloroethyl)amino]-1-methyl-benzimidazol-2-yl]butanoyl]amino]-4-methyl-pentanoate (Example compound 46)
Ethyl (2R)-2-[[(2R)-2-amino-4-[5-[bis(2-chloroethyl)amino]-1-methyl-benzimidazol-2-yl]butanoyl]amino]-4-methyl-pentanoate (Example compound 47)
Ethyl (2S)-2-[[(2S)-2-amino-4-[6-[bis(2-chloroethyl)amino]-3-methyl-imidazo[4,5-b]pyridin-2-yl]butanoyl]amino]-4-methyl-pentanoate (Example compound 48); and
Ethyl (2S)-2-[[(2S)-2-amino-4-[5-[bis(2-chloro-1,1,2,2-tetradeuterio-ethyl)amino]-1-methyl-benzimidazol-2-yl]butanoyl]amino]-4-methyl-pentanoate (Example compound 50)
   or a pharmaceutically acceptable salt, amide or carbamate thereof, including a salt of such a amide or carbamate.

Preferably, the pharmaceutical preparation of the invention comprises a compound of formula (I), or a pharmaceutically acceptable salt, ester, amide or carbamate thereof, or a salt of such an ester, amide or carbamate selected from the group consisting of:
(2S)-2-[[(2S)-2-amino-4-[5-[bis(2-chloroethyl)amino]-1-methyl-benzimidazol-2-yl]butanoyl]amino]-3-methyl-butanoic acid (Example compound 19); and
(2S)-2-[[(2S)-2-amino-4-[5-[bis(2-chloroethyl)amino]-1-methyl-benzimidazol-2-yl]butanoyl]amino]-4-methyl-pentanoic acid (Example compound 20);
(2S)-2-[[(2S)-4-[5-[bis(2-chloroethyl)amino]-1-methyl-benzimidazol-2-yl]-2-(methylamino)butanoyl]amino]-4-methyl-pentanoic acid (Example compound 33)
(2S)-2-[[(2S)-2-Amino-4-methyl-pentanoyl]amino]-4-[5-[bis(2-chloroethyl)amino]-1-methyl-benzimidazol-2-yl]butanoic acid (Example compound 37)
(2S)-2-[[(2S)-2-Amino-4-[5-[bis(2-chloroethyl)amino]-1-methyl-benzimidazol-2-yl]butanoyl]amino]-3-(4-fluorophenyl)propanoic acid (Example compound 39)
(2S)-2-[[(2S)-2-Amino-4-[5-[bis(2-chloroethyl)amino]-1-phenyl-benzimidazol-2-yl]butanoyl]amino]-4-methyl-pentanoic acid (Example compound 40)
2S)-2-[[(2S)-2-amino-3-[5-[bis(2-chloroethyl)amino]-1-methyl-benzimidazol-2-yl]propanoyl]amino]-3-methyl-butanoic acid (Example compound 42)
(2S)-2-[[(2S)-2-amino-4-methyl-pentanoyl]amino]-3-[5-[bis(2-chloroethyl)amino]-1-methyl-benzimidazol-2-yl]propanoic acid (Example compound 43)
(2S)-2-[[(2S)-2-amino-3-[5-[bis(2-chloroethyl)amino]-1-methyl-benzimidazol-2-yl]propanoyl]amino]-4-methyl-pentanoic acid (Example compound 44); and
(2S)-2-[[(2S)-2-amino-4-[5-[bis(2-chloro-1,1,2,2-tetradeuterio-ethyl)amino]-1-methyl-benzimidazol-2-yl]butanoyl]amino]-4-methyl-pentanoic acid (Example compound 51)
   or a salt thereof.

Preferably, the pharmaceutical preparation of the invention comprises a compound of formula (I), or a pharmaceutically acceptable salt, ester, amide or carbamate thereof, or a salt of such an ester, amide or carbamate, wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is formula (VIa):

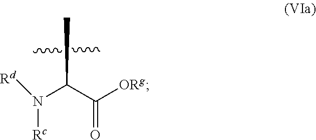

wherein, R^{c} and R^{d} are each independently selected from the group consisting of H, C₁₆alkyl, C(O)C₁alkyl and —CH₂-phenyl, wherein said alkyl or said phenyl is optionally substituted by 1, 2 or 3 groups selected from halogen; and
R^{g} is selected from the group consisting of H and C₁alkyl, wherein said alkyl is optionally substituted by 1, 2 or 3 groups selected from halogen.

Preferably, the pharmaceutical preparation of the invention comprises a compound of formula (Ia) or (Ib), or a pharmaceutically acceptable salt, ester, amide or carbamate thereof, or a salt of such an ester, amide or carbamate,

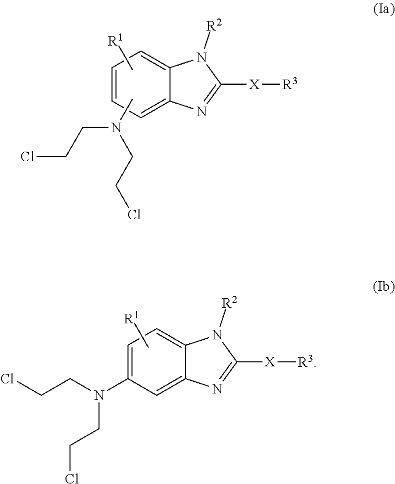

wherein, X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is formula (VIa):

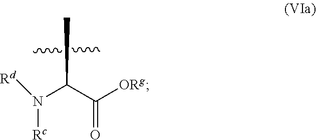

wherein, R^{c} and R^{d} are each independently selected from the group consisting of H, C₁alkyl, C(O)C₁alkyl and —CH₂-phenyl, wherein said alkyl or said phenyl is optionally substituted by 1, 2 or 3 groups selected from halogen;
R^{g} is selected from the group consisting of H and C₁alkyl, wherein said alkyl is optionally substituted by 1, 2 or 3 groups selected from halogen.

Preferably, the pharmaceutical preparation of the invention comprises a compound of formula (I), or a pharmaceutically acceptable salt, ester, amide or carbamate thereof, or a salt of such an ester, amide or carbamate, wherein,
X is C₁₋₆alkylene;
W₁, W₂, W₃ and W₄ are each CH, or one of W₁, W₂, W₃ and W₄ is N and the others are CH;
R¹ is H;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is formula (VIa):

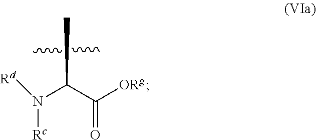

wherein, R^{c} and R^{d} are each H; and
R^{g} is H.

Preferably, the pharmaceutical preparation of the invention comprises a compound of formula (I), or a pharmaceutically acceptable salt, ester, amide or carbamate thereof, or a salt of such an ester, amide or carbamate, wherein, X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of phenyl optionally substituted by 1, 2 or 3 halogens; and C₁₋₆alkyl optionally substituted by 1, 2 or 3 halogens (for example —C₁₋₄alkyl optionally substituted by 1, 2 or 3 halogens, for example methyl); and
R³ is formula (VIa):

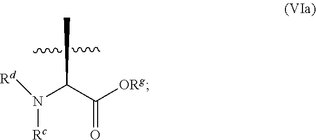

wherein, R^{c} and R^{d} are each H; and
R^{g} is H.

Preferably, the pharmaceutical preparation of the invention comprises a compound of formula (I), or a pharmaceutically acceptable salt, ester, amide or carbamate thereof, or a salt of such an ester, amide or carbamate,
wherein, X is —CH₂— or —CH₂—CH₂—;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is formula (VIa):

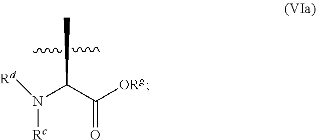

wherein, R^{c} and R^{d} are each H; and
R^{g} is H.

Preferably, the pharmaceutical preparation of the invention comprises a compound according to formula (I), or a salt or solvate thereof, wherein,
X is C₁₋₆alkylene;
W₁, W₂, W₃ and W₄ are each CH, or one of W₁, W₂, W₃ and W₄ is N and the others are CH;
R¹ is selected from the group consisting of H; —C₁₋₄alkyl optionally substituted by 1, 2 or 3 groups independently selected from halogen; and halogen;
R² is selected from the group consisting of H; phenyl optionally substituted by 1, 2 or 3 groups independently selected from halogen; and —C₁₋₆alkyl optionally substituted by 1, 2 or 3 groups independently selected from halogen;
R³ is formula (VI)

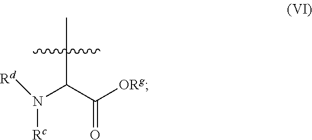

wherein, R^{c} and R^{d} are each independently selected from the group consisting of H, C₁₆alkyl, —C(O)C₁alkyl and —CH₂-phenyl, wherein said alkyl or said phenyl is optionally substituted by 1, 2 or 3 groups selected from halogen; and
R^{g} is selected from the group consisting of H and C₁alkyl, wherein said alkyl is optionally substituted by 1, 2 or 3 groups selected from halogen.

Preferably, the pharmaceutical preparation of the invention comprises a compound of formula (I) or the pharmaceutically acceptable salt, ester, amide or carbamate thereof, or a salt of such an ester, amide or carbamate selected from the group consisting of:
(2S)-2-amino-3-[5-[bis(2-chloroethyl)amino]-1-methyl-benzimidazol-2-yl]propanoic acid (Example compound 17);
(S)-2-amino-4-(5-(bis(2-chloroethyl)amino)-1-methyl-1H-benzo[d]imidazol-2-yl)butanoic acid (Example compound 18);
Ethyl (2S)-2-amino-4-[5-[bis(2-chloroethyl)amino]-1-methyl-benzimidazol-2-yl]butanoate (Example compound 38)
(2S)-2-Amino-4-[5-[bis(2-chloroethyl)amino]-1-phenyl-benzimidazol-2-yl]butanoic acid (Example compound 41); and
(2S)-2-amino-4-[5-[bis(2-chloro-1,1,2,2-tetradeuterio-ethyl)amino]-1-methyl-benzimidazol-2-yl]butanoic acid (Example compound 49);
   or a salt thereof.

Preferably, the pharmaceutical preparation of the invention comprises ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1).

Preferably, the compound of formula (I) or an ester, amide or carbamate thereof is present in the pharmaceutical preparation of the invention as a pharmaceutically acceptable salt, more preferably the compound of formula (I) or an ester, amide or carbamate thereof is present as a hydrochloride salt.

Preferably, the pharmaceutical preparation of the invention comprises Betadex Sulfobutyl Ether Sodium and ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1).

Preferably, the pharmaceutical preparation of the invention comprises compound of formula (I) or the pharmaceutically acceptable salt, ester, amide or carbamate thereof, or a salt of such an ester, amide or carbamate (excluding the mass of any counterion) in an amount of about 1 mg to 150 mg per administration. For example, 1 mg, 2 mg, 3 mg, 5 mg, 10 mg, 15 mg, 20 mg, 25 mg, 40 mg, 50 mg, 60 mg, 70 mg, 80 mg, 90 mg, 100 mg, 110 mg, 120 mg, 130 mg, 140 mg or 150 mg. Alternatively, a compound of the invention, or salt and/or solvate thereof (excluding the mass of any counterion or solvent), may be administered in a single high dose. A single high dose may be about 150 mg to 800 mg. For example, 150 mg, 200 mg, 300 mg, 400 mg, 500 mg, 600 mg, 700 mg or 800 mg.

Preferably, the pharmaceutical preparations of the invention comprise a negatively charged cyclodextrin or a pharmaceutically acceptable derivative of a negatively charged cyclodextrin at a weight ratio (w/w) of the negatively charged cyclodextrin or a pharmaceutically acceptable derivative of a negatively charged cyclodextrin to a compound of formula (I), or a pharmaceutically acceptable salt, ester, amide or carbamate thereof, or a salt of such an ester, amide or carbamate of about 1:1 to about 100:1. Typically, the pharmaceutical preparation comprises a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 4:1 to about 80:1, about 3:1 to about 60:1, about 2:1 to about 50:1, about 4:1 to about 30:1, or about 5:1 to about 20:1.

Preferably, the pharmaceutical preparations of the invention comprise a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 5:1 to about 20:1. In exemplary embodiments, the pharmaceutical preparations of the invention may comprise a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of at least about 5:1. The pharmaceutical preparations of the invention may comprise a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, or about 25:1.

Preferably, the pharmaceutical preparations of the invention comprise a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 5:1, about 10:1, or about 20:1.

Preferably, the pharmaceutical preparation of the invention comprises Betadex Sulfobutyl Ether Sodium and ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1) in a weight ratio of from 5:1 to 20:1.

Preferably, the pharmaceutical preparation of the invention comprises Betadex Sulfobutyl Ether Sodium and ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1) in a weight ratio of 10:1.

In exemplary embodiments, wherein the pharmaceutical preparation of the invention comprises a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 5:1 to about 20:1, the pharmaceutical preparation may comprise, for example, 1 mg to 50 mg of a compound of formula (I), or a pharmaceutically acceptable salt, ester, amide or carbamate thereof, or a salt of such an ester, amide or carbamate (excluding the mass of any counterion) and about 5 mg to about 1000 mg of a negatively charged cyclodextrin or a pharmaceutically acceptable derivative of a negatively charged cyclodextrin. For example, a pharmaceutical preparation comprising 20 mg of a compound of formula (I), or a pharmaceutically acceptable salt, ester, amide or carbamate thereof, or a salt of such an ester, amide or carbamate (excluding the mass of any counterion) may comprise about 100 mg to about 400 mg of a negatively charged cyclodextrin or a pharmaceutically acceptable derivative of a negatively charged cyclodextrin (for example, about 200 mg to about 300 mg of a negatively charged cyclodextrin or a pharmaceutically acceptable derivative of a negatively charged cyclodextrin).

Additional excipients may be included in pharmaceutical preparations of the invention. A further excipient may be included in the pharmaceutical preparation, for example, to improve the dissolution rate of the compound of formula (I), or a pharmaceutically acceptable salt, ester, amide or carbamate thereof, or a salt of such an ester, amide or carbamate into an aqueous solution and/or improve the stability of the compound of formula (I), or a pharmaceutically acceptable salt, ester, amide or carbamate thereof, or a salt of such an ester, amide or carbamate when in its lyophilized form.

The one or more further pharmaceutically acceptable excipients may be selected from the group consisting of polysorbate 80, propylene glycol, sucrose, trehalose dihydrate, MgCl₂, CaCl₂, citric acid, salts of citric acid (such as trisodium citrate dihydrate), lactic acid and salts of lactic acid, tartaric acid and salts of tartaric acid, phosphoric acid and salts of phosphoric acid, glycine, maleic acid, succinic acid and salts of succinic acid, aspartic acid, benzoic acid and salts of benzoic acid. Preferably, the further pharmaceutically acceptable excipients selected from citric acid and salts of citric acid (such as trisodium citrate dihydrate).

Typically, when the pharmaceutical preparation of the invention comprises one or more further excipients in addition to a negatively charged cyclodextrin or a pharmaceutically acceptable derivative of a negatively charged cyclodextrin, the one or more further excipients are each independently present in the preparation at a weight ratio (w/w) of excipient to the compound of formula (I), or a pharmaceutically acceptable salt, ester, amide or carbamate thereof, or a salt of such an ester, amide or carbamate of about 0.1:1 to about 100:1. For example, about 1:1 to about 50:1, for example, about 1:1 to about 30:1, about 1:1 to about 20:1 or about 1:1 to about 10:1, for example about 1.0:1 to about 7.5:1, about 1.0:1 to about 5:1, about 1.0:1 to about 2.0:1.

In certain embodiments, the pharmaceutical preparation of the invention is a lyophilized powder. It has been found that a negatively charged cyclodextrin or a pharmaceutically acceptable derivative of a negatively charged cyclodextrin can be included in a lyophilized formulation of a compound of formula (I), or a pharmaceutically acceptable salt, ester, amide or carbamate thereof, or a salt of such an ester, amide or carbamate. Reconstitution of the lyophilized powder provides an aqueous solution in which compound of formula (I), or a pharmaceutically acceptable salt, ester, amide or carbamate thereof, or a salt of such an ester, amide or carbamate is especially stable.

A pharmaceutical preparation of the invention, when in the form of a lyophilized powder, may essentially consist of a compound formula (I), or a pharmaceutically acceptable salt, ester, amide or carbamate thereof, or a salt of such an ester, amide or carbamate and a negatively charged cyclodextrin or a pharmaceutically acceptable derivative of a negatively charged cyclodextrin. Alternatively, a pharmaceutical preparation of the invention, when in the form of a lyophilized powder, comprises one or more further excipient in addition to the negatively charged cyclodextrin or pharmaceutically acceptable derivative of a negatively charged cyclodextrin. The one or more further excipient(s) may be selected from the group consisting of sucrose, polysorbate 80, propylene glycol, trehalose dihydrate, MgCl₂, CaCl₂, citric acid, or salt of citric acid (such as trisodium citrate dihydrate), lactic acid and salts of lactic acid, tartaric acid and salts of tartaric acid, phosphoric acid and salts of phosphoric acid, glycine, maleic acid, succinic acid and salts of succinic acid, aspartic acid, benzoic acid and salts of benzoic acid. Preferably, the further excipient is selected from citric acid, or salt of citric acid (such as trisodium citrate dihydrate).

Preferably, the pharmaceutical preparation of the invention comprises Betadex Sulfobutyl Ether Sodium, ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1), citric acid and trisodium citric dihydrate.

Preferably, the pharmaceutical preparation of the invention comprises Betadex Sulfobutyl Ether Sodium, ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1), citric acid and trisodium citric dihydrate, wherein the Betadex Sulfobutyl Ether Sodium and ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1) are present in a weight ratio of from 5:1 to 20:1. The further excipient of citric acid and salt of citric acid (such as trisodium citrate dihydrate) is, for example, present in a weight ratio to the compound of formula (I), or a pharmaceutically acceptable salt, ester, amide or carbamate thereof, or a salt of such an ester, amide or carbamate (for example Example compound 1) of about 0.1:1 to about 100:1. For example the ratio may be about 1:1 to about 50:1, for example, about 1:1 to about 30:1, about 1:1 to about 20:1 or about 1:1 to about 10:1, for example about 1.0:1 to about 7.5:1, about 1.0:1 to about 5:1, about 1.0:1 to about 2.0:1.

Preferably, the pharmaceutical preparation of the invention comprises Betadex Sulfobutyl Ether Sodium, ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1), citric acid and trisodium citric dihydrate, wherein the Betadex Sulfobutyl Ether Sodium and ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1) are present in a weight ratio of 10:1. The further excipient of citric acid and salt of citric acid (such as trisodium citrate dihydrate) is, for example, present in a weight ratio to the compound of formula (I), or a pharmaceutically acceptable salt, ester, amide or carbamate thereof, or a salt of such an ester, amide or carbamate (for example Example compound 1) of about 0.1:1 to about 100:1. For example the ratio may be about 1:1 to about 50:1, for example, about 1:1 to about 30:1, about 1:1 to about 20:1 or about 1:1 to about 10:1, for example about 1.0:1 to about 7.5:1, about 1.0:1 to about 5:1, about 1.0:1 to about 2.0:1.

Preferably, in embodiments wherein the pharmaceutical preparation is a lyophilized powder, the pharmaceutical preparation is free, or substantially free, from any organic solvent. By “free” from any organic solvent is it meant that the lyophilized powder pharmaceutical preparation does not contain any measurable amount of an organic solvent. By “substantially free” from any organic solvent it is meant that the lyophilized powder pharmaceutical preparation comprises only trace amounts of an organic solvent, such as less than about a total of about 0.1% w/v of an organic solvent. In certain embodiments, the pharmaceutical preparation of the invention is a liquid pharmaceutical formulation. That is to say that the formulation is in the form of a liquid when stored at room temperature.

The present invention also provides a composition comprising a pharmaceutical preparation of the invention and a physiologically acceptable aqueous solvent or diluent. Preferably, the composition of the invention comprises a compound of formula (I) as defined herein, or pharmaceutically acceptable salt, ester, amide or carbamate thereof, or salt of such an ester, amide or carbamate, a negatively charged cyclodextrin or a pharmaceutically acceptable derivative of a negatively charged cyclodextrin and a physiologically acceptable aqueous solvent. Preferably, the physiologically acceptable aqueous solvent for use in the compositions of the invention is a glucose solution, a saline solution, or a mixture thereof.

The compositions of the invention may be suitable for parenteral administration to a subject. Injection and infusion solutions may be prepared by diluting a lyophilized powder or a liquid pharmaceutical formulation of the invention with one or more physiologically acceptable solvent or diluents. Exemplary solvents or diluents include mannitol, glucose, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

In certain embodiments, the pharmaceutical preparation of the invention is administered to the subject following dilution, for example, with a physiologically acceptable aqueous solvent or diluent such as a saline solution, a glucose solution, or a mixture thereof. Any such solvent or diluent may optionally be buffered.

Preferably, the physiologically acceptable solvent is an aqueous solution such as a glucose solution (for example, a 5% glucose solution (w/v in water)), a saline solution (for example, a 0.9% sodium chloride solution (w/v in water)), or a mixture thereof. A pharmaceutical preparation of the invention may be diluted with a physiologically acceptable solvent, for example, to a concentration of the compound of formula (I), or a pharmaceutically acceptable salt, ester, amide or carbamate thereof, or a salt of such an ester, amide or carbamate (excluding the mass of any counterion) of about 0.001 mg/mL to about 3 mg/mL (for example, 0.01 mg/mL to 2 mg/mL, 0.05 mg/mL to 1.4 mg/mL, 0.05 mg/mL to 1.2 mg/mL, 0.05 mg/mL to 1 mg/mL, 0.05 mg/mL to 0.9 mg/mL, 0.05 mg/mL to 0.8 mg/mL 0.1 mg/mL to 0.4 mg/mL, or 0.1 mg/mL to 0.3 mg/mL), before administration to a subject.

Compositions suitable for parenteral administration of the invention are preferably suitable for administration by infusion or injection, and especially suitable for administration by intravenous infusion.

In certain embodiments, injection and infusion solutions of a compound of formula (I), or a pharmaceutically acceptable salt, ester, amide or carbamate thereof, or a salt of such an ester, amide or carbamate may be prepared by diluting a lyophilized powder of the compound of formula (I), or a pharmaceutically acceptable salt, ester, amide or carbamate thereof, or a salt of such an ester, amide or carbamate that does not contain a negatively charged cyclodextrin or a pharmaceutically acceptable derivative of a negatively charged cyclodextrin with one or more physiologically acceptable solvents or diluents that contain a negatively charged cyclodextrin or a pharmaceutically acceptable derivative of a negatively charged cyclodextrin. For example, the compound of formula (I), or a pharmaceutically acceptable salt, ester, amide or carbamate thereof, or a salt of such an ester, amide or carbamate may be a lyophilized powder comprising (or consisting essentially of) a compound of formula (I) or a pharmaceutically acceptable salt, ester, amide or carbamate thereof, or a salt of such an ester, amide or carbamate.

In embodiments wherein the one or more physiologically acceptable solvents or diluents contain a negatively charged cyclodextrin or a pharmaceutically acceptable derivative of a negatively charged cyclodextrin, the negatively charged cyclodextrin or pharmaceutically acceptable derivative of a negatively charged cyclodextrin may be present at a concentration of about 0.1 mg/mL to 10 mg/mL. For example, the negatively charged cyclodextrin or pharmaceutically acceptable derivative of a negatively charged cyclodextrin is present at a concentration of about 0.1 mg/mL to 3 mg/mL, for example about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.5 mg/mL, 2 mg/mL, 2.5 mg/mL, 3 mg/mL, 4 mg/mL, or 5 mg/mL.

Alternatively or additionally, a negatively charged cyclodextrin or a pharmaceutically acceptable derivative of a negatively charged cyclodextrin may be added directly to the injection or infusion solution following the reconstitution and/or dilution of a compound of formula (I), or a pharmaceutically acceptable salt, ester, amide or carbamate thereof, or a salt of such an ester, amide or carbamate with a suitable physiologically acceptable solvent or diluent; or a compound of formula (I), or a pharmaceutically acceptable salt, ester, amide or carbamate thereof, or a salt of such an ester, amide or carbamate may be added directly to the injection or infusion solution following the reconstitution and/or dilution of a negatively charged cyclodextrin or a pharmaceutically acceptable derivative of a negatively charged cyclodextrin with a suitable physiologically acceptable solvent or diluent. Thus, the present invention also provides an injection or infusion solution comprising a compound of formula (I), or a pharmaceutically acceptable salt, ester, amide or carbamate thereof, or a salt of such an ester, amide or carbamate, a negatively charged cyclodextrin or a pharmaceutically acceptable derivative of a negatively charged cyclodextrin, one or more physiologically acceptable solvent or diluent, and optionally one or more further excipient.

In embodiments wherein a negatively charged cyclodextrin or a pharmaceutically acceptable derivative of a negatively charged cyclodextrin is added directly to the injection or infusion solution following the reconstitution and/or dilution of a compound of formula (I), or a pharmaceutically acceptable salt, ester, amide or carbamate thereof, or a salt of such an ester, amide or carbamate with a suitable physiologically acceptable solvent or diluent; or a compound of formula (I), or a pharmaceutically acceptable salt, ester, amide or carbamate thereof, or a salt of such an ester, amide or carbamate is added directly to the injection or infusion solution following the reconstitution and/or dilution of a negatively charged cyclodextrin or a pharmaceutically acceptable derivative of a negatively charged cyclodextrin with a suitable physiologically acceptable solvent or diluent, the compound of formula (I), or a pharmaceutically acceptable salt, ester, amide or carbamate thereof, or a salt of such an ester, amide or carbamate may be in the form of a pharmaceutical preparation, for example a lyophilized powder comprising a compound of formula (I), or a pharmaceutically acceptable salt, ester, amide or carbamate thereof, or a salt of such an ester, amide or carbamate.

In embodiments that provide an injection or infusion solution comprising a compound of formula (I), or a pharmaceutically acceptable salt, ester, amide or carbamate thereof, or a salt of such an ester, amide or carbamate, a negatively charged cyclodextrin or a pharmaceutically acceptable derivative of a negatively charged cyclodextrin, one or more physiologically acceptable solvent or diluent, and optionally one or more further excipient, the injection or infusion solution preferably comprise a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 1:1 to about 100:1, for example a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 4:1 to about 80:1, or about 2:1 to about 50:1. In one embodiment, the injection or infusion solution comprises a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or pharmaceutically acceptable salt, ester, amide or carbamate thereof, or salt of such an ester, amide or carbamate of about 4:1 to about 80:1, or about 5:1 to about 20:1. For example, the injection or infusion solution comprises a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 5:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, or about 25:1. In one preferred embodiment, the weight ratio (w/w) of a negatively charged cyclodextrin or a pharmaceutically acceptable derivative of a negatively charged cyclodextrin to a compound of formula (I), or a pharmaceutically acceptable salt, ester, amide or carbamate thereof, or a salt of such an ester, amide or carbamate is about 5:1, about 10:1, or about 20:1.

### Additional Therapeutic Agents

Whilst a compound of formula ( ), or a salt thereof, may be present in the pharmaceutical preparation of the invention as the sole active ingredient, it is also possible for the pharmaceutical preparation of the invention to additionally contain one or more additional therapeutic agent(s). Such agents are known in the art. Examples of further therapeutic agents for use in the invention include steroids (prednisone, prednisolone and dexamethasone), IMiDs (thalidomide, lenalidomide and pomalidomide), PIs (bortezomib, carfilzomib and ixazomib), histone deacetylase (HDAC) inhibitors (panobinostat), conventional chemotherapy (alkylators (e.g. melflufen, melphalan, cyclophosphamide, bendamustine), doxorubicin), checkpoint inhibitors, nuclear transport inhibitors (selinexor), anti-apoptotic inhibitors, adoptive cell therapy, bi-specific T-cell engagers (BiTEs), B-cell targeting agents, and monoclonal antibodies.

Thus, in certain embodiments, the pharmaceutical preparation of the invention comprises a compound of formula (I), or a pharmaceutically acceptable salt, ester, amide or carbamate thereof, or a salt of such an ester, amide or carbamate, a negatively charged cyclodextrin or a pharmaceutically acceptable derivative of a negatively charged cyclodextrin, and one or more additional therapeutic agent(s), optionally selected from steroids (prednisone, prednisolone and dexamethasone), IMiDs (thalidomide, lenalidomide and pomalidomide), PIs (bortezomib, carfilzomib and ixazomib), histone deacetylase (HDAC) inhibitors (panobinostat), conventional chemotherapy (alkylators (e.g. melflufen, melphalan, cyclophosphamide, bendamustine), doxorubicin), checkpoint inhibitors, nuclear transport inhibitors (selinexor), anti-apoptotic inhibitors, adoptive cell therapies, bi-specific T-cell engagers (BiTEs), B-cell targeting agents, and monoclonal antibodies. Preferably, the pharmaceutical preparation of the invention comprises a compound of formula (I), or a pharmaceutically acceptable salt, ester, amide or carbamate thereof, or a salt of such an ester, amide or carbamate, a negatively charged cyclodextrin or a pharmaceutically acceptable derivative of a negatively charged cyclodextrin, and one or more additional therapeutic agent(s), selected from the group consisting of a proteasome inhibitor (PI), an immunomodulatory drug (IMiD), a B-cell targeting agent, a steroid, a histone deacetylase (HDAC) inhibitor, and a monoclonal antibody.

More preferably, the pharmaceutical preparation of the invention comprises a compound of formula (I), or a pharmaceutically acceptable salt, ester, amide or carbamate thereof, or a salt of such an ester, amide or carbamate, a negatively charged cyclodextrin or a pharmaceutically acceptable derivative of a negatively charged cyclodextrin, and one or more additional therapeutic agent(s) selected from the group consisting of a proteasome inhibitor (PI), a steroid, and a monoclonal antibody.

For example, the pharmaceutical preparation of the invention may consist essentially of a compound of formula (I), or a pharmaceutically acceptable salt, ester, amide or carbamate thereof, or a salt of such an ester, amide or carbamate, a negatively charged cyclodextrin or a pharmaceutically acceptable derivative of a negatively charged cyclodextrin, and one or more additional therapeutic agent(s). Or, for example, the pharmaceutical preparation of the invention may consist essentially of a compound of formula (I), or a pharmaceutically acceptable salt, ester, amide or carbamate thereof, or a salt of such an ester, amide or carbamate, a negatively charged cyclodextrin or a pharmaceutically acceptable derivative of a negatively charged cyclodextrin, one or more further excipient, and one or more additional therapeutic agent(s).

The precise quantity and concentration of the additional therapeutic agent that may be present in a pharmaceutical formulation of the invention may vary with the dosing schedule, the potency of the particular agent chosen, the age, size, sex and condition of the subject (typically a mammal, for example a human), the nature and severity of the disease or condition, and other relevant medical and physical factors. The skilled person can readily determine the quantity and concentration of the compound of formula (I), or a pharmaceutically acceptable salt, ester, amide or carbamate thereof, or a salt of such an ester, amide or carbamate, and optional one or more additional therapeutic agent(s) suitable for use according to the invention.

### Treatments

The pharmaceutical preparations, compositions and kits of the invention find use as medicaments.

In certain embodiments, the pharmaceutical preparations, compositions and kits of the invention find use in the treatment and/or prophylaxis of diseases or conditions that may be treated with conventional chemotherapy agents, for example, with an alkylator (e.g. melfufen, melphalan, cyclophosphamide and bendamustine).

In certain embodiments, the pharmaceutical preparations and compositions of the invention find use in the treatment and/or prophylaxis of cancer, reducing tumour growth, killing tumour cells and/or the treatment and/or prophylaxis of amyloidosis. For example, the pharmaceutical preparations and compositions of the invention find use in the treatment and/or prophylaxis of cancer, reducing tumour growth and/or killing tumour cells. Thus, the pharmaceutical preparations and compositions of the invention may be used for curing and/or prolonging the survival of patients afflicted with cancer diseases.

In embodiments, the pharmaceutical preparation, compositions, and kits of the invention find use in the treatment or prophylaxis of cancer. Particular examples of cancers that may be treated or prevented by administering a compound or composition of the invention include carcinoma, a sarcoma, a myeloma, a leukemia, a lymphoma or a mixed type of cancer. Exemplary cancers that may be treated or prevented by administering a compound or composition of the invention include, but are not limited to, hematologic/blood cell cancers such as a leukemia (for example, acute lymphoblastic leukemia including adult and childhood acute lymphoblastic leukemia; acute myeloid leukemia including adult and childhood acute myeloid leukemia; chronic lymphocytic leukemia such as B Cell chronic lymphocytic leukemia; chronic myelogenous leukemia; and hairy cell leukemia); lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; primary mediastinal large B cell lymphoma; mantle cell lymphoma; diffuse large B cell lymphoma; and primary central nervous system lymphoma); and other hematologic cancers (for example, chronic myeloproliferative disorders; multiple myeloma/plasma cell neoplasm; myelodysplastic syndromes; and myelodysplastic/myeloproliferative disorders); osteosarcoma, ovarian cancer, breast cancer; lung cancer; glioblastoma; retinoblastoma and metastases of the aforementioned cancers.

The pharmaceutical preparation, compositions, and kits of the invention are particularly cytotoxic towards hematological cancer cells. Thus, the pharmaceutical preparation, compositions, and kits of the invention are particularly beneficial in the treatment or prophylaxis of one or more of the aforementioned hematological cancers.

In embodiments, when the pharmaceutical preparation, compositions, and kits of the invention are used in the treatment or prophylaxis of cancer (e.g. selected from hematologic/blood cell cancers such as a leukemia (for example, acute lymphoblastic leukemia including adult and childhood acute lymphoblastic leukemia; acute myeloid leukemia including adult and childhood acute myeloid leukemia; chronic lymphocytic leukemia such as B Cell chronic lymphocytic leukemia; chronic myelogenous leukemia; and hairy cell leukemia); lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; primary mediastinal large B cell lymphoma; mantle cell lymphoma; diffuse large B cell lymphoma; and primary central nervous system lymphoma); and other hematologic cancers (for example, chronic myeloproliferative disorders; multiple myeloma/plasma cell neoplasm; myelodysplastic syndromes; and myelodysplastic/myeloproliferative disorders); osteosarcoma, ovarian cancer, breast cancer; lung cancer; glioblastoma; retinoblastoma and metastases of the aforementioned cancers), the preparation or composition is administered (for example simultaneously, sequentially or separately) with one or more additional therapeutic agent(s), for example a steroid, an MiD, a PI, a HDAC inhibitor, conventional chemotherapy, a checkpoint inhibitor, a nuclear transport inhibitor, an anti-apoptotic inhibitor, an adoptive cell therapy, a BiTE, a B-cell targeting agents, and a monoclonal antibody.

The amount of the compound of formula (I), or a pharmaceutically acceptable salt, ester, amide or carbamate thereof, or a salt of such an ester, amide or carbamate which is required to achieve a therapeutic effect will vary with particular route of administration and the characteristics of the subject under treatment, for example the species, age, weight, sex, medical conditions, the particular disease and its severity, and other relevant medical and physical factors. An ordinarily skilled physician can readily determine and administer the effective amount of the compound of formula (I), or a pharmaceutically acceptable salt, ester, amide or carbamate thereof, or a salt of such an ester, amide or carbamate required for treatment or prophylaxis of cancer. The pharmaceutical preparations and compositions of the invention find utility in a method for treating a subject which comprises administering a pharmaceutically effective amount of a pharmaceutical preparation or composition of the invention to a subject. Typically, a pharmaceutical preparation of the invention is administered following dilution into physiologically acceptable solvent or diluent, such as a saline solution or glucose solution.

### Unit Doses

The pharmaceutical preparation of the invention may be provided as unit doses. Preferred unit doses are those containing a requisite dose of a compound of formula (I) or a pharmaceutically acceptable salt, ester, amide or carbamate thereof, or a salt of such an ester, amide or carbamate, suitable for use according to the invention.

In preferred embodiments, the pharmaceutical preparation of the invention may be provided in a vial containing a unit dose of a compound of formula (I) or a pharmaceutically acceptable salt, ester, amide or carbamate thereof, or a salt of such an ester, amide or carbamate. For example, the pharmaceutical preparation of the invention may be provided in a vial containing a unit dose of 1 to 800 mg of a compound of formula (I) or a pharmaceutically acceptable salt, ester, amide or carbamate thereof, or a salt of such an ester, amide or carbamate (excluding the mass of any counterion), for example, the vial may contain a unit dose of 1 mg, 2 mg, 3 mg, 5 mg, 10 mg, 15 mg, 20 mg, 25 mg, 40 mg, 50 mg, 60 mg, 70 mg, 80 mg, 90 mg, 100 mg, 110 mg, 120 mg, 130 mg, 140 mg, 150 mg, 200 mg, 300 mg, 400 mg, 500 mg, 600 mg, 700 mg or 800 mg of a compound of formula (I) or a pharmaceutically acceptable salt, ester, amide or carbamate thereof, or a salt of such an ester, amide or carbamate (excluding the mass of any counterion).

### Kits

The invention provides a kit suitable for the preparation of a pharmaceutical preparation according to the invention. A kit of the invention comprises a compound of formula (I), or pharmaceutically acceptable salt, ester, amide or carbamate thereof, or salt of such an ester, amide or carbamate, and a negatively charged cyclodextrin or a pharmaceutically acceptable derivative of a negatively charged cyclodextrin. The compound of formula (I), or a pharmaceutically acceptable salt, ester, amide or carbamate thereof, or a salt of such an ester, amide or carbamate and a negatively charged cyclodextrin or a pharmaceutically acceptable derivative of a negatively charged cyclodextrin in the kit may be provided together as a mixture or they may be provided separately, for example in separate vials that are later combined prior to use of the kit (for example combined to prepare a pharmaceutical preparation or composition of the invention prior to use in a method or use of the invention defined herein).

In certain embodiments, the kit of the invention additionally comprises one or more physiologically acceptable aqueous solvent or diluent, such as a glucose solution or saline solution. Typically the physiologically acceptable aqueous solvent or diluent is provided separately to the compound of formula (I), or a pharmaceutically acceptable salt, ester, amide or carbamate thereof, or a salt of such an ester, amide or carbamate and negatively charged cyclodextrin or pharmaceutically acceptable derivative of a negatively charged cyclodextrin in the kit; or the physiologically acceptable aqueous solvent or diluent is provided together as a mixture with the negatively charged cyclodextrin or pharmaceutically acceptable derivative of a negatively charged cyclodextrin in the kit and the compound of formula (I), or a pharmaceutically acceptable salt, ester, amide or carbamate thereof, or a salt of such an ester, amide or carbamate is provided separately, for example the compound of formula (I), or a pharmaceutically acceptable salt, ester, amide or carbamate thereof, or a salt of such an ester, amide or carbamate is provided in a separate vial that is later combined with a composition comprising the negatively charged cyclodextrin or pharmaceutically acceptable derivative of a negatively charged cyclodextrin and physiologically acceptable aqueous solvent or diluent prior to use of the kit (for example combined to prepare a composition of the invention prior to use in a method or use of the invention defined herein).

In one embodiment of the invention, the kit of the invention additionally comprises one or more further therapeutic agents as described herein. The one or more one or more further therapeutic agents in the kit may be provided together as a mixture with one or both of a compound of formula (I), or a pharmaceutically acceptable salt, ester, amide or carbamate thereof, or a salt of such an ester, amide or carbamate and a negatively charged cyclodextrin or a pharmaceutically acceptable derivative of a negatively charged cyclodextrin, or may be provided separately.

Preferably, the kit of the invention comprises a compound of formula (I) as described herein, or pharmaceutically acceptable salt, ester, amide or carbamate thereof, or salt of such an ester, amide or carbamate, as a lyophilised preparation comprising (or consisting essentially of) compound of formula (I) as described herein, or pharmaceutically acceptable salt, ester, amide or carbamate thereof, or salt of such an ester, amide or carbamate and sucrose.

For the avoidance of doubt, the compound of formula (I), or pharmaceutically acceptable salt, ester, amide or carbamate thereof, or salt of such an ester, amide or carbamate thereof, negatively charged cyclodextrin or pharmaceutically acceptable derivative of a negatively charged cyclodextrin, optional physiologically acceptable aqueous solvent or diluent, optional one or more pharmaceutically acceptable organic solvent, and optional one or more further therapeutic agent, are present in a kit according to the invention in a form and quantity suitable for the preparation of a pharmaceutical preparation according to the invention. The skilled person can readily determine a quantity of the compound of formula (I), or pharmaceutically acceptable salt, ester, amide or carbamate thereof, or salt of such an ester, amide or carbamate, pharmaceutically acceptable organic solvent, and optional one or more further therapeutic agents, suitable for the use according to the invention.

The invention also provides kits suitable for the preparation of injection and infusion solutions comprising a compound of formula (I), or a pharmaceutically acceptable salt, ester, amide or carbamate thereof, or a salt of such an ester, amide or carbamate and a negatively charged cyclodextrin or a pharmaceutically acceptable derivative of a negatively charged cyclodextrin. Such a kit comprises compound of formula (I), or pharmaceutically acceptable salt, ester, amide or carbamate thereof, or salt of such an ester, amide or carbamate, a negatively charged cyclodextrin or a pharmaceutically acceptable derivative of a negatively charged cyclodextrin, and one or more physiologically acceptable aqueous solvent or diluent, such as a glucose solution or saline solution. The compound of formula (I), or pharmaceutically acceptable salt, ester, amide or carbamate thereof, or salt of such an ester, amide or carbamate, negatively charged cyclodextrin or a pharmaceutically acceptable derivative of a negatively charged cyclodextrin, and one or more physiologically acceptable aqueous solvent or diluent may be provided as a mixture or separately. For example, the compound of formula (I), or a pharmaceutically acceptable salt, ester, amide or carbamate thereof, or a salt of such an ester, amide or carbamate and the negatively charged cyclodextrin or pharmaceutically acceptable derivative of a negatively charged cyclodextrin may be provided together as a mixture and the one or more physiologically acceptable aqueous solvent or diluent may be provided separately. Alternatively, the negatively charged cyclodextrin or pharmaceutically acceptable derivative of a negatively charged cyclodextrin and one or more one or more physiologically acceptable aqueous solvent or diluent may be provided as a mixture (i.e. provided as a composition comprising the negatively charged cyclodextrin or a pharmaceutically acceptable derivative of a negatively charged cyclodextrin and one or more one or more physiologically acceptable aqueous solvent or diluent) and the compound of formula (I), or a pharmaceutically acceptable salt, ester, amide or carbamate thereof, or a salt of such an ester, amide or carbamate may be provided separately.

Preferably the pharmaceutically acceptable aqueous solvent is 5% glucose solution (w/v in water) or a 0.9% sodium chloride solution (w/v in water). Preferably, such solutions are suitable for intravenous injection and/or infusion to a subject.

In such kits, preferably the compound of formula (I), or pharmaceutically acceptable salt, ester, amide or carbamate thereof, or salt of such an ester, amide or carbamate, and a negatively charged cyclodextrin or a pharmaceutically acceptable derivative of a negatively charged cyclodextrin are provided as a pharmaceutical preparation of the invention, for example a lyophilized powder of the invention or a liquid pharmaceutical formulation of the invention. As such, the invention provides a kit suitable for the preparation of injection and infusion solutions comprising a pharmaceutical preparation of the invention (for example a lyophilized powder of the invention), and one or more physiologically acceptable aqueous solvent or diluent, such as a glucose solution or saline solution, as described herein.

Alternatively, the invention provides a kit suitable for the preparation of injection and infusion solutions comprising a pharmaceutical preparation comprising compound of formula (I), or pharmaceutically acceptable salt, ester, amide or carbamate thereof, or salt of such an ester, amide or carbamate (for example a lyophilized pharmaceutical preparation comprising a compound of formula (I), or pharmaceutically acceptable salt, ester, amide or carbamate thereof, or salt of such an ester, amide or carbamate, and optionally sucrose), a composition comprising a negatively charged cyclodextrin or a pharmaceutically acceptable derivative of a negatively charged cyclodextrin, and one or more physiologically acceptable aqueous solvent or diluent, such as a glucose solution or saline solution, as described herein.

Alternatively, the invention provides a kit suitable for the preparation of injection and infusion solutions comprising a pharmaceutical preparation comprising a compound of formula (I), or pharmaceutically acceptable salt, ester, amide or carbamate thereof, or salt of such an ester, amide or carbamate (for example a lyophilized pharmaceutical preparation comprising a compound of formula (I), or pharmaceutically acceptable salt, ester, amide or carbamate thereof, or salt of such an ester, amide or carbamate, and optionally sucrose), and a composition comprising a negatively charged cyclodextrin or a pharmaceutically acceptable derivative of a negatively charged cyclodextrin and one or more physiologically acceptable aqueous solvent or diluent, such as a glucose solution or saline solution, as described herein.

In one embodiment of the invention, the kit of the invention suitable for the preparation of injection and infusion solutions additionally comprises one or more further therapeutic agents as described herein.

Preferably, the kit of the invention comprises instructions, for example instructions that instruct a user to admix a stated amount of a compound of formula (I), or pharmaceutically acceptable salt, ester, amide or carbamate thereof, or salt of such an ester, amide or carbamate with a stated amount of a negatively charged cyclodextrin or a pharmaceutically acceptable derivative of a negatively charged cyclodextrin.

Such instructions may also provide guidance on the storage conditions and/or administration instructions.

For the avoidance of doubt, the compound of formula (I), or pharmaceutically acceptable salt, ester, amide or carbamate thereof, or salt of such an ester, amide or carbamate, negatively charged cyclodextrin or pharmaceutically acceptable derivative of a negatively charged cyclodextrin, one or more physiologically acceptable aqueous solvent or diluent, and optional one or more further therapeutic agent, are present in a kit according to the invention in a form and quantity suitable for the preparation of a solution suitable for use as an injection or infusion solution. The skilled person can readily determine a quantity of the compound of formula (I), or pharmaceutically acceptable salt, ester, amide or carbamate thereof, or salt of such an ester, amide or carbamate, negatively charged cyclodextrin or pharmaceutically acceptable derivative of a negatively charged cyclodextrin, physiologically acceptable aqueous solvent, and optional one or more further therapeutic agents, suitable for the use according to the invention.

Finally, in embodiments of the invention wherein a kit is provided, a kit of the invention may comprise a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 1:1 to about 100:1, for example a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 4:1 to about 80:1, about 2:1 to about 50:1, or about 5:1 to about 20:1, wherein the amount of the compound of formula (I), or a pharmaceutically acceptable salt, ester, amide or carbamate thereof, or a salt of such an ester, amide or carbamate is the amount present in the kit. For example, the kits of the invention may comprise a negatively charged cyclodextrin or a pharmaceutically acceptable derivative of a negatively charged cyclodextrin at a weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate of about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, or about 25:1. In one preferred embodiment, the weight ratio (w/w) of the negatively charged cyclodextrin or pharmaceutically acceptable derivative of a negatively charged cyclodextrin to a compound of formula (I), or a pharmaceutically acceptable salt, ester, amide or carbamate thereof, or a salt of such an ester, amide or carbamate is about 5:1, about 10:1, or about 20:1, wherein the amount of the compound of formula (I), or a pharmaceutically acceptable salt, ester, amide or carbamate thereof, or a salt of such an ester, amide or carbamate is the amount present in the kit.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. The applicant reserves the right physically to incorporate into this application any and all materials and information from any such articles, patents, patent applications, or other physical and electronic documents.

The following Examples illustrate the invention.

### EXAMPLES

The following Example Compounds and Biological Examples are all disclosed in International Patent Application No. PCT/EP2022/066756 which was published under the number WO2022/263679. The Example Preparations illustrate the pharmaceutical preparations as claimed herein.

### Example Compounds

The compounds as described herein may be prepared according to known methods for those skilled in the art. Other reaction schemes, as well as a variety of different solvents, temperatures and other reaction conditions, could be readily devised by those skilled in the art.

Compounds of Formula (I) as described hereinabove may be prepared according to the protocols described in International Patent Application No. PCT/EP2022/066756 which was published under the number WO2022/263679. The synthetic methods for Example compounds 1 to 51 as described on pages 37 to 132 of WO2022/263679 are incorporated herein by reference. For completeness, the structures of the compounds are shown in Table 1 below.

### BIOLOGICAL EXAMPLES

### Biological Example 1—Assessment of In Vitro Cytotoxic Potency in a MM.1S Cell Viability Assay

Methods: MM.1S Cells were cultivated in RPMI medium 1640 (IX) supplemented with 10 μg/μL penicillin/streptomycin, 10% heat inactivated FBS, 0.1 mM Sodium Pyruvate, 1×MEM NEAA (ThermoFisher) and 1 mM HEPES. Cells were grown to reach log phase before being exposed to the test compounds.

Test compounds (Example compounds 1 to 36, 38 to 41 and 45 to 48) and comparative compounds (bendamustine, busulfan, melphalan, chlorambucil and 4-hydroperoxy cyclophosphamide) were dissolved in 100% DMSO and added to a 384 assay plate. Additional DMSO was added to maintain the same final DMSO concentration (0.2%) in all wells. 25 μL cell suspension with a concentration of 100000 cells/mL was added to each well. The cells were cultured in a CO₂ incubator at 37° C. for 72 h. Plates were equilibrated to room temperature for 30 minutes before adding 20 μL CellTiter-Glo® 2.0 (Promega) reagent to all wells. The plate was then allowed to stabilize for 20 minutes before recording the luminescence (emission filter 700 nm) in an Envision plate reader. The plate reader data was normalized to negative controls (no treatment) vs positive controls (cells treated with 400 μM Chlorpromazine for 72 h) and IC₅₀ was derived from a 4 Parameter Logistic regression curve fitting.

Results: Test compounds were evaluated on their ability to inhibit MM.1S cell proliferation and cause cytotoxicity in vitro. Dose responses could be extracted from experiments and are shown in Table 1 below. The test compounds displayed good cytotoxicity towards MM.1S cells. Example compounds 1 to 16, 21 to 32, 34 to 36, 38 and 45 to 48 were found to be more cytotoxic than bendamustine, busulfan, melphalan, chlorambucil and 4-hydroperoxy cyclophosphamide in this assay.

TABLE 1
Cytotoxicity in MM.1S cells

| | | | MM.1S, | |
|---|---|---|---|---|
| | | | IC50, | |
| Example | | MM.1S IC50 | st dev | MM.1S, |
| No | Structure | (μM) | (μM) | n |
| 1 | 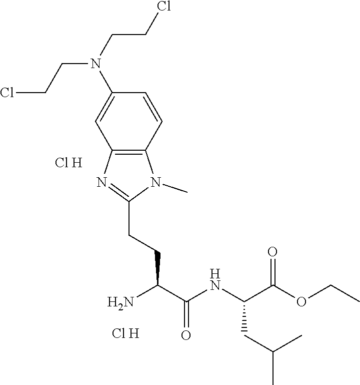 | 0.0068 | 0.0022 | 20 |
| 2 | 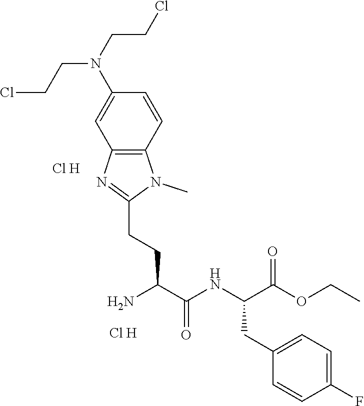 | 0.0078 | 0.0029 | 7 |
| 3 | 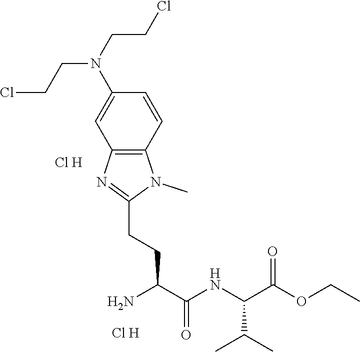 | 0.0068 | 0.0012 | 2 |
| 4 | 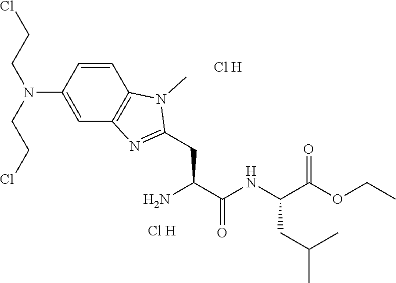 | 0.0066 | 0.0017 | 2 |
| 5 | 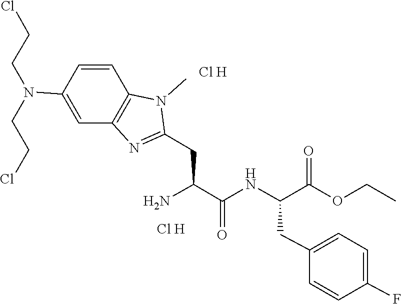 | 0.0226 | 0.0079 | 2 |
| 6 | 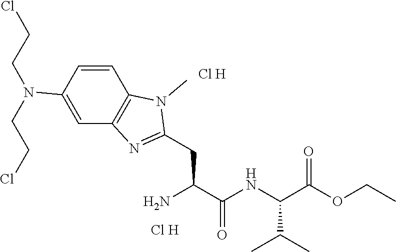 | 0.0987 | 0.0381 | 4 |
| 7 | 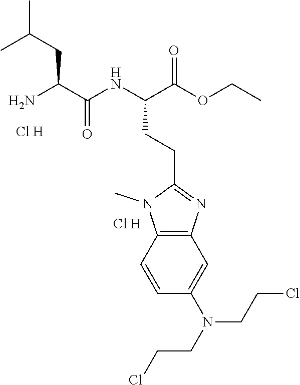 | 0.0229 | 0.0108 | 2 |
| 8 | 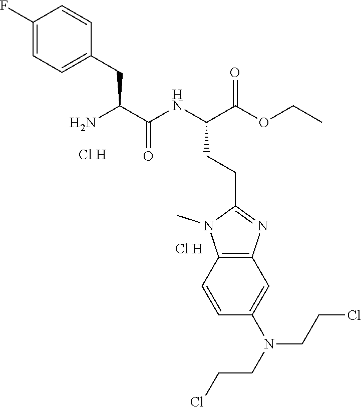 | 0.0144 | 0.0031 | 2 |
| 9 | 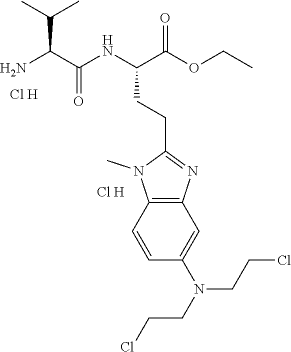 | 0.0225 | 0.0025 | 2 |
| 10 | 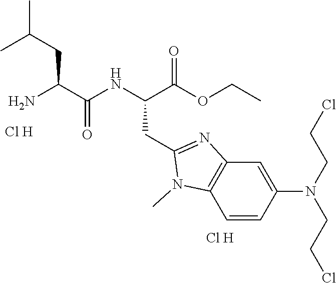 | 0.0192 | 0.0037 | 8 |
| 11 | 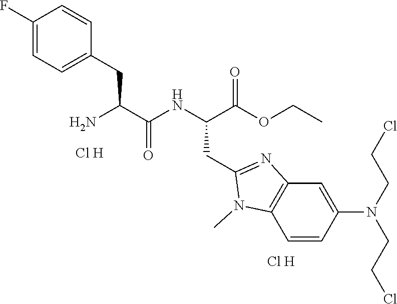 | 0.0147 | 0.0011 | 2 |
| 12 | 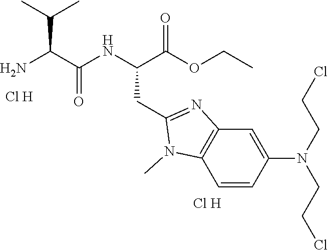 | 0.0125 | 0.0045 | 2 |
| 13 | 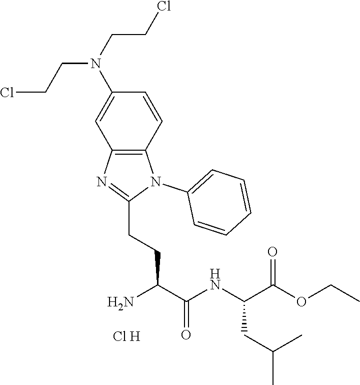 | 0.049 | 0.0249 | 7 |
| 14 | 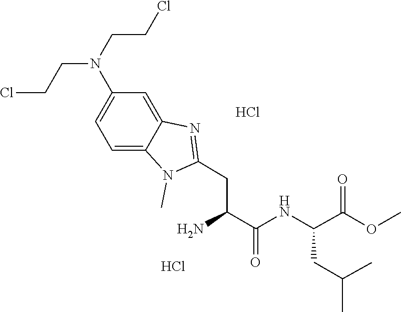 [SMILES: COC(=O)[C@H](CC(C)C)NC(=O)[C@@H](N)Cc1nc2cc(N(CCCl)CCCl)ccc2n1C ; Cl ; Cl] | 0.0087 | 0.0012 | 2 |
| 15 | 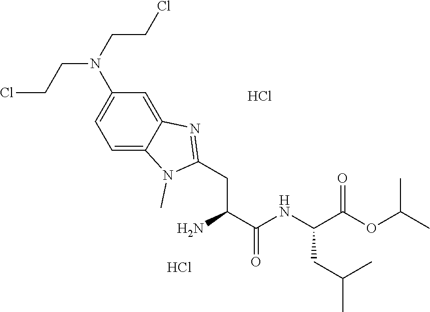 [SMILES: CC(C)C[C@H](NC(=O)[C@@H](N)Cc1nc2cc(N(CCCl)CCCl)ccc2n1C)C(=O)OC(C)C ; Cl ; Cl] | 0.0091 | 0.0007 | 2 |
| 16 | 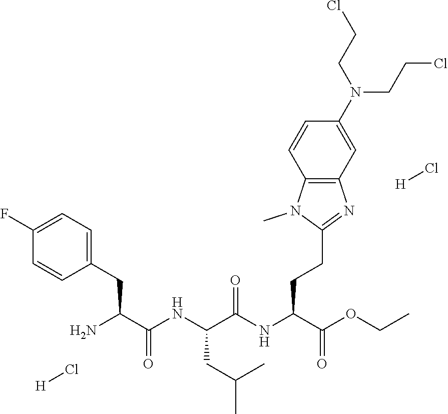 | 0.025 | 0.0076 | 2 |
| 17 | 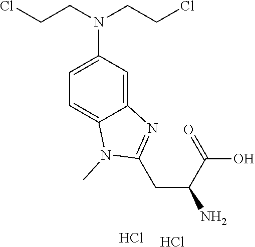 [SMILES: Cn1c(C[C@H](N)C(=O)O)nc2cc(N(CCCl)CCCl)ccc21 ; Cl ; Cl] | 39.967 | 4.5325 | 2 |
| 18 | 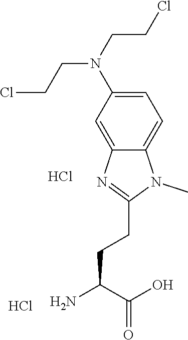 [SMILES: Cn1c(CC[C@H](N)C(=O)O)nc2cc(N(CCCl)CCCl)ccc21 ; Cl ; Cl] | 20.244 | 20.968 | 14 |
| 19 | 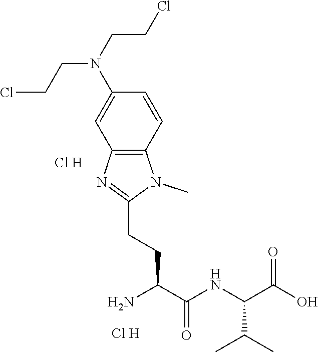 | 4.0199 | 0.6051 | 2 |
| 20 | 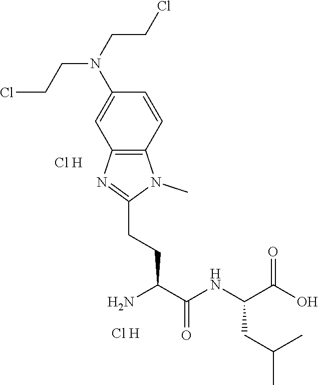 | 5.753 | 1.149 | 6 |
| 21 | 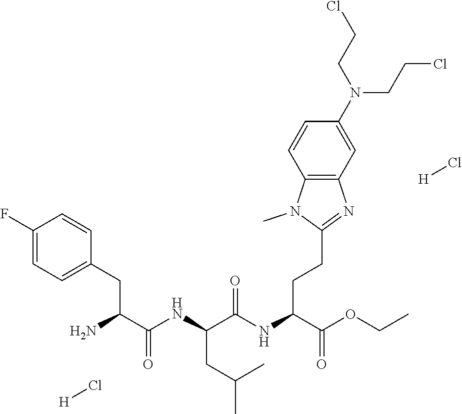 [SMILES: CCOC(=O)[C@H](CCc1nc2cc(N(CCCl)CCCl)ccc2n1C)NC(=O)[C@@H](CC(C)C)NC(=O)[C@@H](N)Cc1ccc(F)cc1 ; Cl ; Cl] | 0.0266 | 0.0016 | 3 |
| 22 | 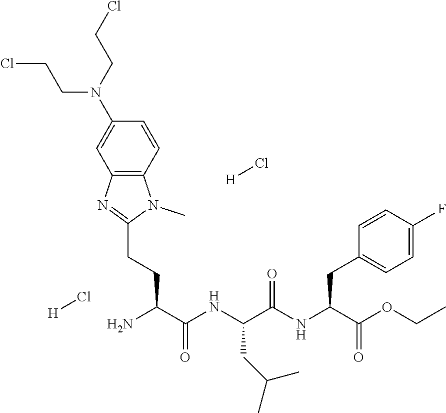 [SMILES: CCOC(=O)[C@H](Cc1ccc(F)cc1)NC(=O)[C@H](CC(C)C)NC(=O)[C@@H](N)CCc1nc2cc(N(CCCl)CCCl)ccc2n1C ; Cl ; Cl] | 0.0313 | 0.0018 | 2 |
| 23 | 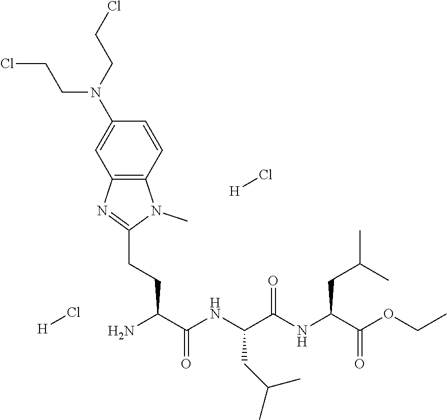 [SMILES: CCOC(=O)[C@H](CC(C)C)NC(=O)[C@H](CC(C)C)NC(=O)[C@@H](N)CCc1nc2cc(N(CCCl)CCCl)ccc2n1C ; Cl ; Cl] | 0.0418 | 0.0017 | 2 |
| 24 | 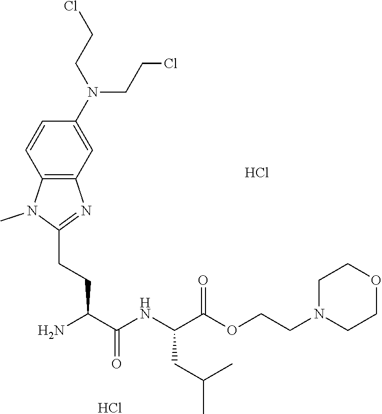 [SMILES: CC(C)C[C@H](NC(=O)[C@@H](N)CCc1nc2cc(N(CCCl)CCCl)ccc2n1C)C(=O)OCCN1CCOCC1 ; Cl ; Cl] | 0.1561 | 0.0247 | 2 |
| 25 | 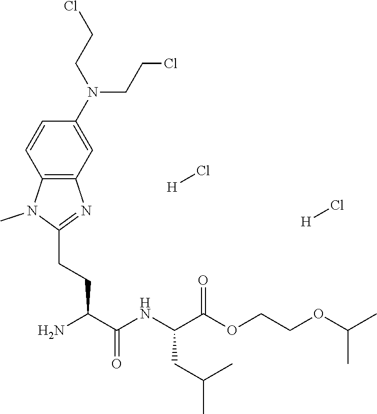 [SMILES: CC(C)C[C@H](NC(=O)[C@@H](N)CCc1nc2cc(N(CCCl)CCCl)ccc2n1C)C(=O)OCCOC(C)C ; Cl ; Cl] | 0.0185 | 0.002 | 3 |
| 26 | 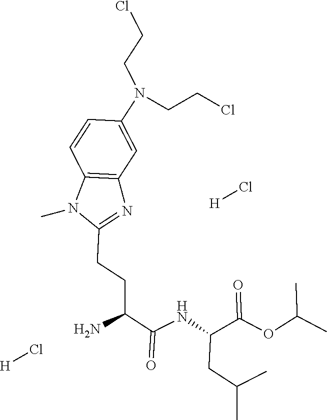 [SMILES: Cl ; CC(C)C[C@H](NC(=O)[C@@H](N)CCc1nc2cc(N(CCCl)CCCl)ccc2n1C)C(=O)OC(C)C ; Cl] | 0.0068 | 0.0019 | 2 |
| 27 | 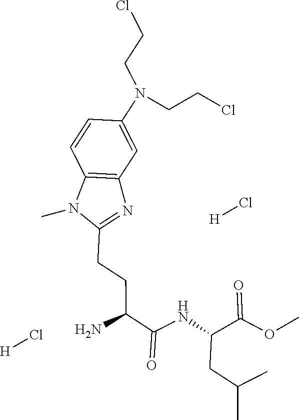 | 0.0082 | 0.0014 | 2 |
| 28 | 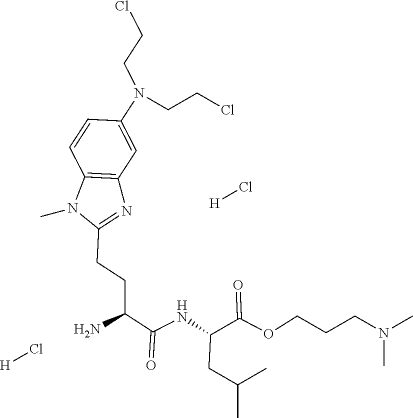 | 0.352 | — | 1 |
| 29 | 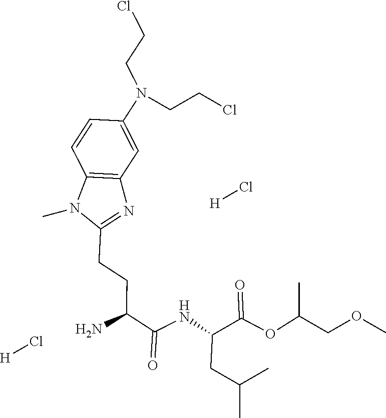 | 0.0014 | — | 1 |
| 30 | 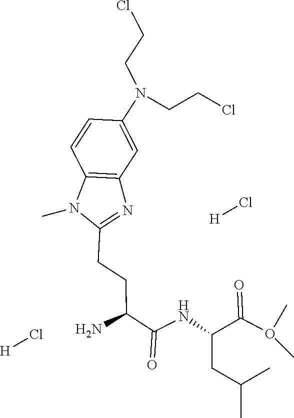 | 0.0397 | 0.0081 | 4 |
| 31 | 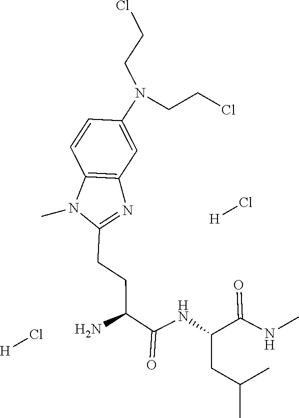 | 0.181 | 0.008 | 2 |
| 32 | 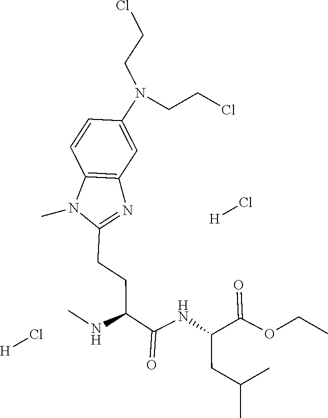 | 0.0143 | 0.002 | 3 |
| 33 | 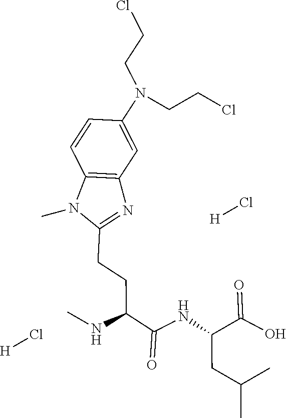 | 79.872 | 13.613 | 2 |
| 34 | 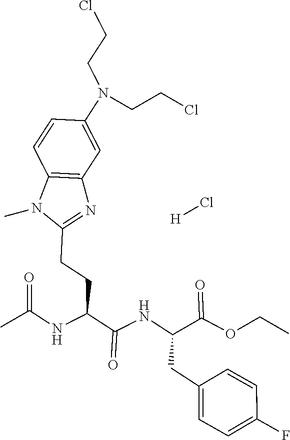 [SMILES: CCOC(=O)[C@H](Cc1ccc(F)cc1)NC(=O)[C@H](CCc1nc2cc(N(CCCl)CCCl)ccc2n1C)NC(C)=O ; Cl] | 0.0683 | 0.0087 | 8 |
| 35 | 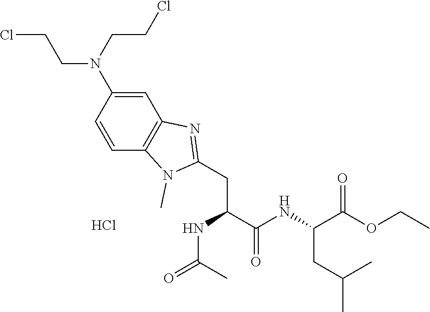 [SMILES: CCOC(=O)[C@H](CC(C)C)NC(=O)[C@H](Cc1nc2cc(N(CCCl)CCCl)ccc2n1C)NC(C)=O ; Cl] | 0.0867 | 0.0087 | 2 |
| 36 | 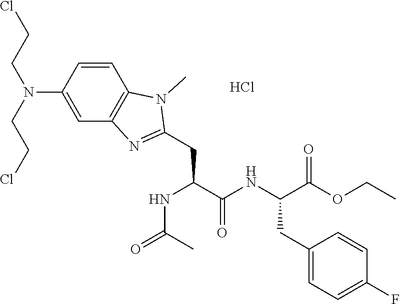 [SMILES: CCOC(=O)[C@H](Cc1ccc(F)cc1)NC(=O)[C@H](Cc1nc2cc(N(CCCl)CCCl)ccc2n1C)NC(C)=O ; Cl] | 0.0575 | 0.0047 | 2 |
| 38 | 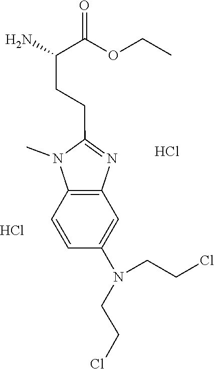 [SMILES: Cl ; CCOC(=O)[C@@H](N)CCc1nc2cc(N(CCCl)CCCl)ccc2n1C ; Cl] | 0.052 | 0.0158 | 7 |
| 39 | 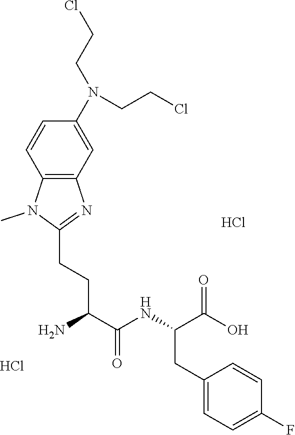 [SMILES: Cn1c(CC[C@H](N)C(=O)N[C@@H](Cc2ccc(F)cc2)C(=O)O)nc2cc(N(CCCl)CCCl)ccc21 ; Cl ; Cl] | >1.996 | — | 1 |
| 40 | 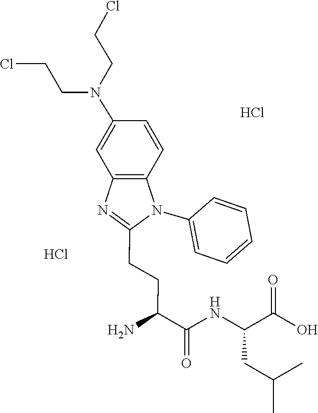 [SMILES: CC(C)C[C@H](NC(=O)[C@@H](N)CCc1nc2cc(N(CCCl)CCCl)ccc2n1-c1ccccc1)C(=O)O ; Cl ; Cl] | 7.177 | 0.709 | 2 |
| 41 | 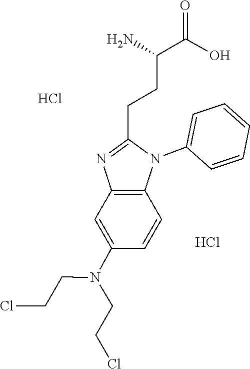 [SMILES: N[C@@H](CCc1nc2cc(N(CCCl)CCCl)ccc2n1-c1ccccc1)C(=O)O ; Cl ; Cl] | 16.507 | 4.3134 | 2 |
| 45 | 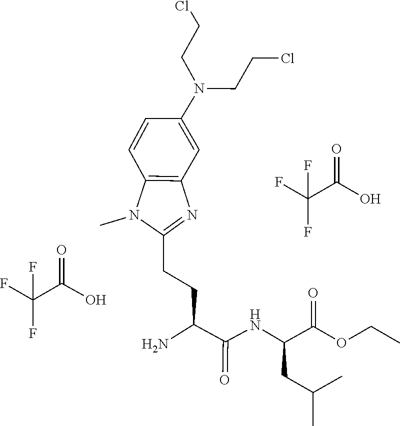 | 0.512 | 0.1113 | 3 |
| 46 | 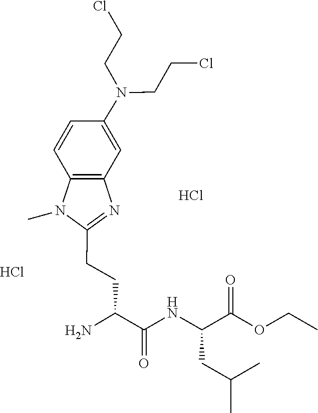 [SMILES: Cl ; CCOC(=O)[C@H](CC(C)C)NC(=O)[C@H](N)CCc1nc2cc(N(CCCl)CCCl)ccc2n1C ; Cl] | 0.0591 | 0.0137 | 3 |
| 47 | 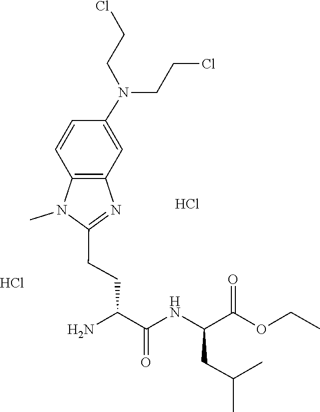 [SMILES: Cl ; CCOC(=O)[C@@H](CC(C)C)NC(=O)[C@H](N)CCc1nc2cc(N(CCCl)CCCl)ccc2n1C ; Cl] | 1.7124 | 0.7595 | 3 |
| 48 | 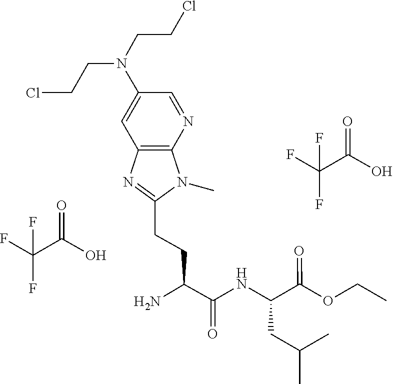 [SMILES: O=C(O)C(F)(F)F ; CCOC(=O)[C@H](CC(C)C)NC(=O)[C@@H](N)CCc1nc2cc(N(CCCl)CCCl)cnc2n1C ; O=C(O)C(F)(F)F] | 0.205 | 0.0262 | 2 |
| Bendamustine | 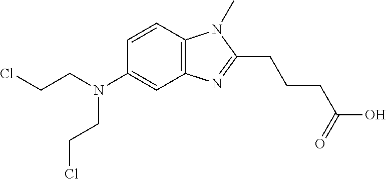 [SMILES: Cn1c(CCCC(=O)O)nc2cc(N(CCCl)CCCl)ccc21] | 23.963 | 6.7392 | 12 |
| Melphalan | 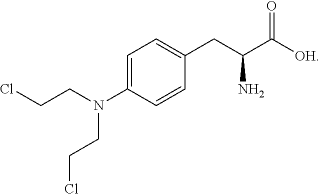 [SMILES: N[C@@H](Cc1ccc(N(CCCl)CCCl)cc1)C(=O)O] | 4.2451 | 0.7017 | 10 |
| Busulfan | 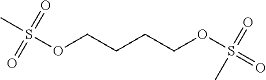 | 180.3 | — | 1 |
| Chlorambucil | 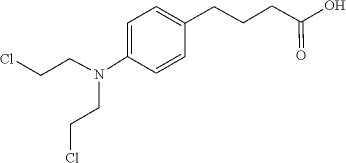 [SMILES: O=C(O)CCCc1ccc(N(CCCl)CCCl)cc1] | 6.873 | 6.4333 | 2 |
| 4-hydroperoxy- cyclophosphamide | 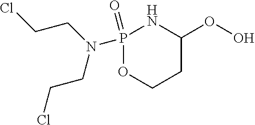 [SMILES: O=P1(N(CCCl)CCCl)NC(OO)CCO1] | 7.2953 | — | 1 |

### Biological Example 2—Assessment of In Vitro Cytotoxicity in Haematological Cell Lines, Human Peripheral Blood Mononuclear Cells and Normal Fibroblasts

Methods: Cell lines used for these experiments are listed in Table 2 below. All cells were cultivated in RPMI medium 1640 (IX). All media was supplemented with 10 μg/μl penicillin/streptomycin, 10% heat inactivated FBS, 0.1 mM Sodium Pyruvate, 1×MEM NEAA (ThermoFisher) and 1 mM HEPES. The DERL-2 cells had higher FBS (20%) and 40 ng/mL IL-2 was added. hPBMCs were thawed and allowed to acclimatise overnight and then stimulated with 10 g/mL PHA-M and 40 ng/mL IL-2 for 4 hours before being exposed to test compounds. All cells, except for hPBMCs, were grown to reach log phase before being exposed to the test compounds.

TABLE 2
Cell lines used in in vitro cytotoxicity experiments

| Cell line ID | Description |
|---|---|
| U937 | Histiocytic Lymphoma |
| THP-1 | Acute Myeloid Leukemia |
| DOHH-2 | B Cell Lymphoma |
| HG-3 | Chronic Lymphocytic Leukemia (CLL) |
| SC-1 | B cell Lymphoma |
| SU-DHL-8 | B-cell non-Hodgkin Lymphoma |
| Toledo | Diffuse Large B Cell Lymphoma |
| DERL-2 | T cell Lymphoma |
| SUP-T1 | T cell Lymphoma |
| AMO-1 | Multiple Myeloma |
| EJM | Multiple Myeloma |
| KMS-12-BM | Multiple Myeloma |
| MM.1S | Multiple Myeloma |
| OPM-2 | Multiple Myeloma |
| RPMI8226 | Multiple Myeloma (resistant) |
| U266 | Multiple Myeloma |
| SK-MM-1 | Plasma Cell Leukemia |
| hPBMC | Human Peripheral blood mononuclear cells |
| BJ | Fibroblasts (normal cells) |

Test compounds were dissolved in 100% DMSO and added to a 384 assay plate. Additional DMSO was added to maintain the same final DMSO concentration (0.2%) in all wells. 25 μL cell suspension with a concentration of 100000 cells/mL was added to each well. The cells were cultured in a CO₂ incubator at 37° C. for 72 h. Plates were equilibrated to room temperature for 30 minutes before adding 20 μL CellTiter-Glo® 2.0 (Promega) reagent to all wells. The plate was then allowed to stabilize for 20 minutes before recording the luminescence (Em filter 700 nm) in an Envision plate reader. The plate reader data was normalized to negative controls (no treatment) vs positive controls (cells treated with 400 μM Chlorpromazine for 72 h) and IC₅₀ was derived from a 4 parameter Logistic regression curve fitting.

Results: Test compounds were evaluated on their ability to inhibit cell proliferation in vitro using several different cell lines of haematological origin. Cytotoxicity was also evaluated in non-malignant human peripheral blood mononuclear cells (hPBMC) and fibroblasts (BJ). Table 3 shows the dose responses extracted from the experiments for all compounds tested. In the table, the results are given as Cytotoxicity IC50 μM+/−standard deviation (number of tests). nd=values not determined.

TABLE 3a
Cytotoxicity of test compounds in normal and cancerous cell lines

| Compound: | 1 | 2 | 10 | 13 |
|---|---|---|---|---|
| THP-1 | 0.016 +/− | nd | nd | nd |
| | 0 (1) | | | |
| U937 | 0.0098 +/− | 0.0142 +/− | 0.0194 +/− | nd |
| | 0.0025 (4) | 0.0042 (2) | 0.0049 (2) | |
| AMO-1 | 0.0497 +/− | nd | nd | 0.9646 +/− |
| | 0.0196 (3) | | | 0.2196 (3) |
| EJM | 0.0271 +/− | nd | nd | 0.356 +/− |
| | 0.0059 (2) | | | 0.0451 (2) |
| KMS-12-BM | 0.0079 +/− | nd | nd | 0.1373 +/− |
| | 0.002 (2) | | | 0.0142 (2) |
| MM.1S | 0.0068 +/− | 0.0078 +/− | 0.0192 +/− | 0.049 +/− |
| | 0.0022 (20) | 0.0029 (7) | 0.0037 (8) | 0.0249 (7) |
| OPM-2 | 0.1734 +/− | nd | nd | 2.1102 +/− |
| | 0.0576 (2) | | | 0.6334 (2) |
| RPMI-8226 | 0.0906 +/− | 0.0415 +/− | 0.3352 +/− | 1.7213 +/− |
| | 0.0549 (6) | 0.004 (2) | 0.0102 (2) | 0.391 (2) |
| SK-MM-1 | 0.0347 +/− | 0.0383 +/− | 0.0624 +/− | nd |
| | 0.024 (5) | 0.0316 (3) | 0.0288 (2) | |
| U266 | 0.0214 +/− | nd | nd | 0.4002 +/− |
| | 0.0085 (2) | | | 0.136 (2) |
| DOHH-2 | 0.0042 +/− | 0.0053 +/− | 0.0639 +/− | nd |
| | 0.0005 (4) | 0.0007 (2) | 0.0237 (2) | |
| HG-3 | 0.0257 +/− | 0.0257 +/− | 0.0713 +/− | nd |
| | 0.0052 (4) | 0.0103 (2) | 0.0069 (2) | |
| SC-1 | 0.0133 +/− | 0.021 +/− | 0.1686 +/− | 0.1344 +/− |
| | 0.0051 (5) | 0.0044 (2) | 0.0874 (2) | 0 (1) |
| SU-DHL-8 | 0.0199 +/− | 0.0204 +/− | 0.0221 +/− | nd |
| | 0.0032 (4) | 0.0009 (2) | 0.0054 (2) | |
| Toledo | 0.0045 +/− | 0.0066 +/− | 0.0085 +/− | nd |
| | 0.0026 (4) | 0.0021 (2) | 0.0003 (2) | |
| DERL-2 | 0.006 +/− | 0.0078 +/− | 0.0226 +/− | nd |
| | 0.0007 (3) | 0.0011 (3) | 0.0058 (3) | |
| SUP-T1 | 0.0127 +/− | 0.0178 +/− | 0.0356 +/− | nd |
| | 0.004 (3) | 0.0053 (3) | 0.0076 (3) | |
| BJ | 1.0745 +/− | nd | nd | >1.996 +/− |
| | 0 (1) | | | (1) |
| hPBMC | 0.0054 +/− | 0.0075 +/− | 0.0264 +/− | nd |
| | 0.0021 (2) | 0.002 (2) | 0.0097 (2) | |

TABLE 3b

| Compound: | 30 | 34 | 38 | Bendamustine | Melphalan |
|---|---|---|---|---|---|
| THP-1 | nd | nd | nd | 40.204 +/− | 4.3058 +/− |
| | | | | 28.995 (2) | 0 (1) |
| U937 | nd | 0.3966 +/− | 0.0253 +/− | 27.061 +/− | nd |
| | | 0.0382 (2) | 0.0008 (2) | 7.409 (3) | |
| AMO-1 | 0.6528 +/− | 0.7576 +/− | nd | 56.977 +/− | 8.3424 +/− |
| | 0.0666 (3) | 0.1497 (3) | | 8.7068 (4) | 1.8996 (3) |
| EJM | 0.1734 +/− | 0.6368 +/− | nd | 64.64 +/− | 40.017 +/− |
| | 0.0191 (2) | 0.0192 (2) | | 2.4078 (3) | 1.2365 (2) |
| KMS-12- | 0.045 +/− | 0.1046 +/− | nd | 27.469 +/− | 9.829 +/− |
| BM | 0.0164 (2) | 0.0047 (2) | | 6.1077 (3) | 1.1877 (2) |
| MM.1S | 0.0397 +/− | 0.0683 +/− | 0.052 +/− | 23.963 +/− | 4.2451 +/− |
| | 0.0081 (4) | 0.0087 (8) | 0.0158 (7) | 6.7392 (12) | 0.7017 (10) |
| OPM-2 | 0.3218 +/− | 0.9609 +/− | nd | 81.878 +/− | 10.176 +/− |
| | 0.0557 (2) | 0.336 (2) | | 7.837 (3) | 1.7559 (2) |
| RPMI-8226 | 0.4924 +/− | 1.4476 +/− | 0.6764 +/− | 111.12 +/− | 32.486 +/− |
| | 0.0701 (2) | 0.2263 (2) | 0.1445 (2) | 34.705 (10) | 3.8841 (2) |
| SK-MM-1 | nd | 0.6039 +/− | 0.1752 +/− | 73.498 +/− | nd |
| | | 0.4592 (3) | 0.0525 (2) | 21.704 (3) | |
| U266 | 0.1314 +/− | 0.2661 +/− | nd | 83 +/− | 18.85 +/− |
| | 0.0024 (2) | 0.1386 (2) | | 41.993 (3) | 3.4229 (2) |
| DOHH-2 | nd | 0.5428 +/− | 0.0281 +/− | 10.701 +/− | nd |
| | | 0.1497 (2) | 0.0099 (2) | 5.9963 (2) | |
| HG-3 | nd | 0.8877 +/− | 0.0498 +/− | 25.787 +/− | nd |
| | | 0.1628 (2) | 0.0236 (2) | 13.589 (2) | |
| SC-1 | 0.0537 +/− | 1.0624 +/− | 0.0772 +/− | 25.141 +/− | 8.6422 +/− |
| | 0 (1) | 0.2814 (3) | 0.0116 (2) | 7.3961 (3) | 0 (1) |
| SU-DHL-8 | nd | 0.2022 +/− | 0.0714 +/− | 14.264 +/− | nd |
| | | 0.1099 (2) | 0.0244 (2) | 3.6275 (3) | |
| Toledo | nd | 0.3276 +/− | 0.0162 +/− | 12.519 +/− | nd |
| | | 0.0368 (2) | 0.0047 (2) | 6.2427 (3) | |
| DERL-2 | nd | nd | nd | 21.071 +/− | 1.8822 +/− |
| | | | | 3.1768 (3) | 0.3506 (3) |
| SUP-T1 | nd | nd | nd | 33.527 +/− | 2.9645 +/− |
| | | | | 11.372 (3) | 0.371 (3) |
| BJ | >1.996 +/− | >1.996 +/− | nd | >598.8 +/− | 103.64 +/− |
| | (1) | (1) | | (2) | 38.599 (2) |
| hPBMC | nd | nd | nd | 15.803 +/− | 3.6018 +/− |
| | | | | 6.6175 (2) | 1.8923 (2) |

It is seen that the compounds of formula (I) are consistently more cytotoxic than the two compounds of the prior art.

### Biological Example 3—Assessment of Cellular Retention/Efflux of Example 18 (after Treatment with Example 1), Bendamustine and Melphalan

Methods: MM.1S cells at 2×10⁶ cells/mL were seeded at 250 μL per well in a 96-well plate. Cells were incubated in the presence of a test compound for 5, 15, and 60 min before pelleting cells by centrifugation at +4° C. for 5 min. Cell media was immediately harvested to a second plate and stored at −80° C. until further analysis. Pelleted cells were washed once with ice cold PBS before storage at −80° C. until further analysis.

Cells were lysed and protein in the media were precipitated using acetonitrile dimethylformamide, 9:1 containing internal standard. Samples were centrifuged and the supernatant was analyzed with LC-MS/MS (ACQUITY UPLC-Xevo TQ-S micro) to determine compound concentration.

Example compound 1, bendamustine and melphalan were used as test compounds.

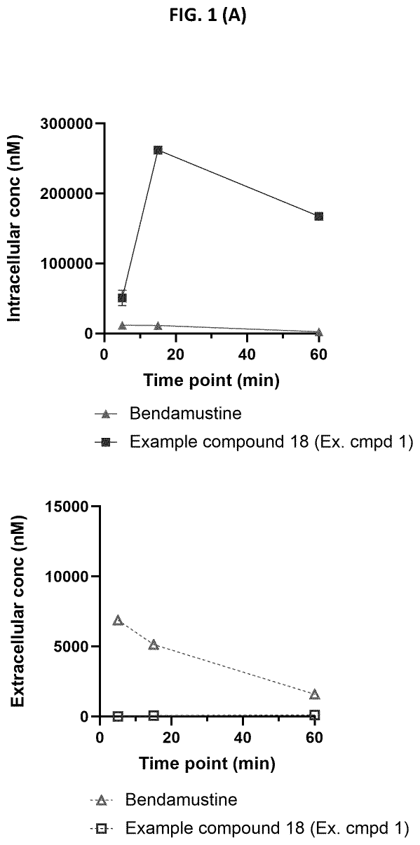

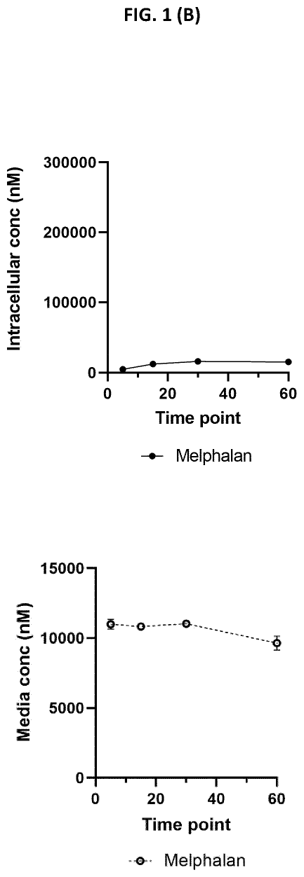

Results: The results are shown in FIG. 1. FIG. 1(A) shows the intracellular and extracellular concentrations of bendamustine and the metabolite compound formed from Example compound 1 (Example compound 18). It is seen that Example compound 18 is rapidly formed inside the cells after treatment with Example compound 1 and low levels of it are found in the media outside the cells. These results indicate that the hydrolysis of Example compound 1 took place inside the cells and that the metabolite of Example compound 1 was retained in the cells. Low intracellular concentrations could be detected of bendamustine and the level decreases over time. The extracellular concentration of bendamustine was quite high and decreased over time.

In FIG. 1(B), there are shown the intracellular and extracellular concentrations of melphalan over the time course of the experiment. Following in vitro treatment of MM.1S cells with melphalan, a low intracellular concentration of melphalan was detected. The extracellular concentration of melphalan was high and constant throughout the experiment.

### Biological Example 4—In Vivo Assessment of Test Compound Cytotoxicity in a Chicken Embryo Xenograft Model

Methods: Inoculation of Fertilized White Leghorn eggs was performed with 3×10⁶ SU-DHL-4 tumour cells on day 9 (post-fertilisation). Viable eggs were injected with 100 μL of either Example compound 1 (8.4 μM (0.008 mg/kg), 33.5 μM (0.033 mg/kg), 167.5 μM (0.164 mg/kg)), bendamustine (8.4 μM (0.005 mg/kg), 33.5 μM (0.02 mg/kg), 167.5 μM (0.1 mg/kg)) or vehicle on day 11, 13, 15 and 17. On day 18, tumours were removed and weighed.

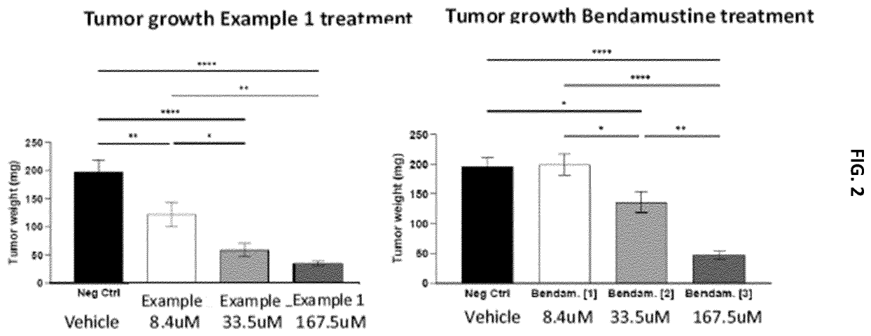

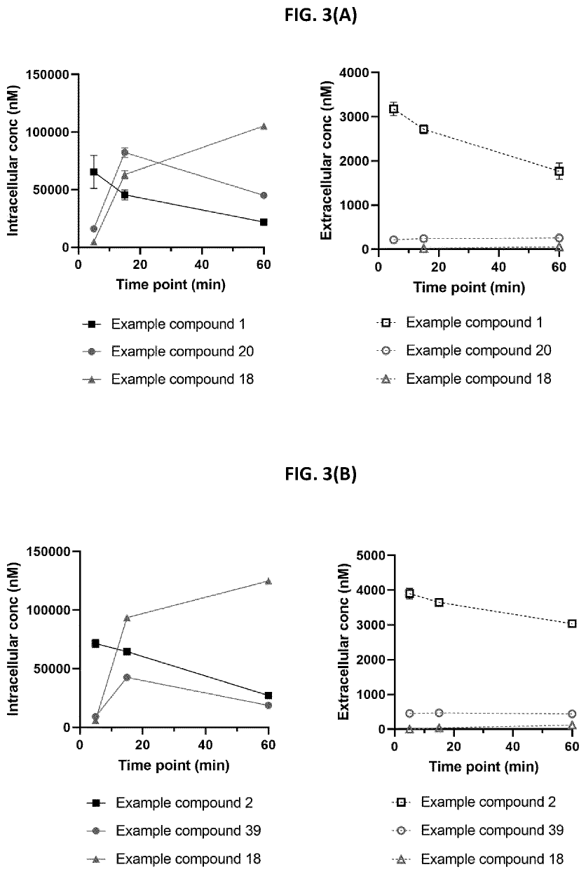

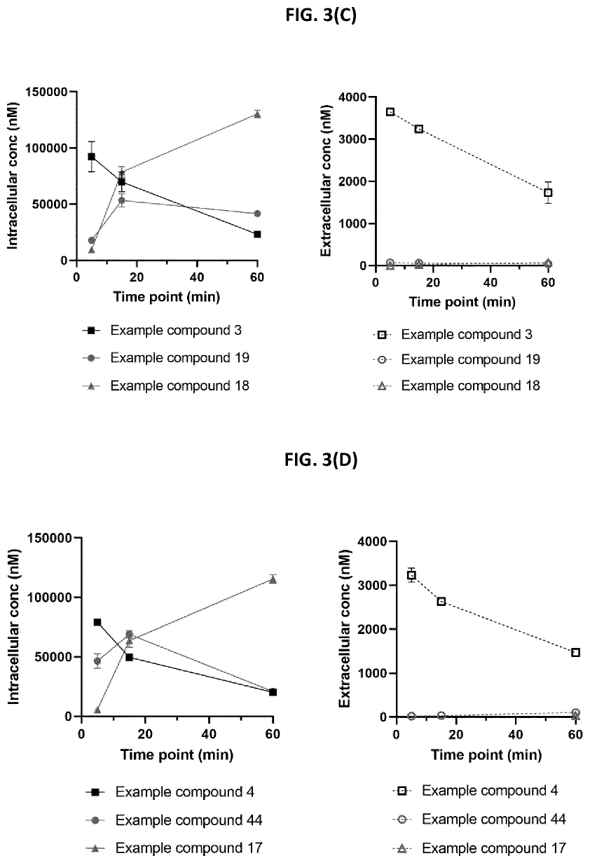

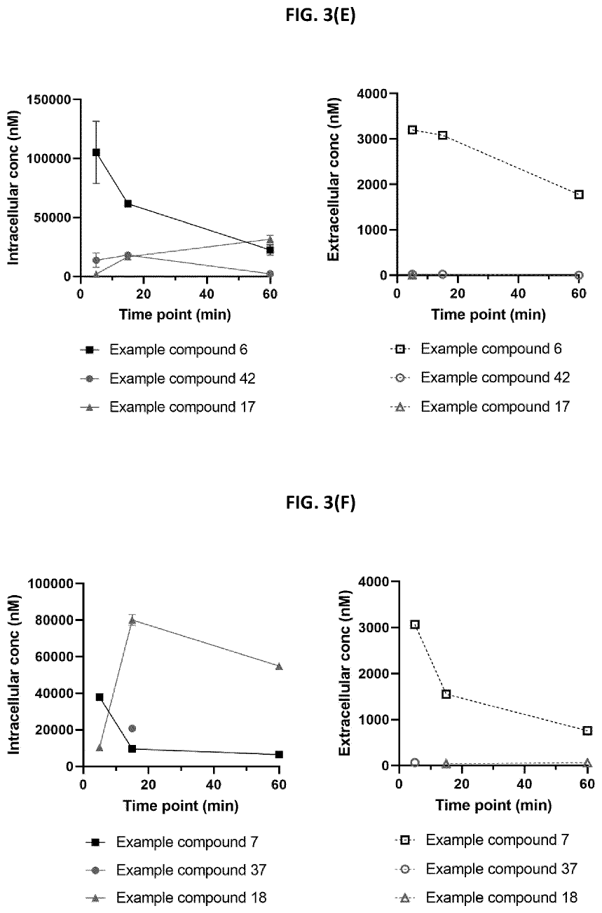

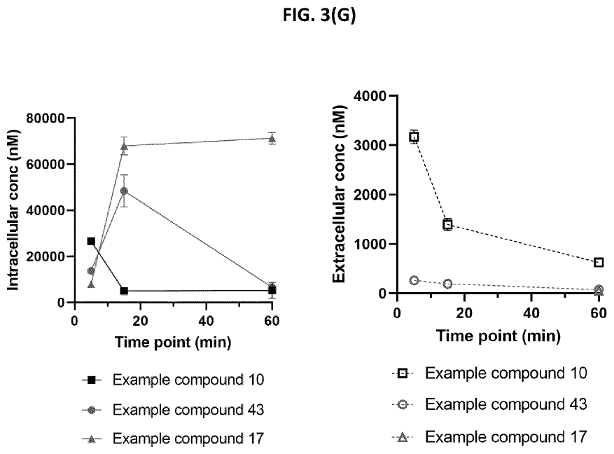

Results: A dose dependent effect on tumour weight was apparent for Example compound 1 (FIG. 2). For Example compound 1, tumour reduction compared to vehicle was 38%, 70% and 82% at 8.4 μM, 33.5 μM and 167.5 μM, respectively. For bendamustine, tumour reduction compared to vehicle was only apparent at 33.5 μM and 167.5 μM, at 30% and 76% reduction respectively. Thus, Example compound 1 shows potent tumour cytotoxicity in this model.

### Biological Example 5—Assessment of Cellular Retention/Efflux of PDC Compounds and Metabolites

Method: Method described in Biological example 3.

Example compounds 1, 2, 3, 4, 6, 7 and 10 were investigated. Intracellular metabolites are formed from all compounds tested. The compound used for cell treatment was metabolised to the ester-hydrolysed compound (here called Metabolite A) and to the amide-hydrolysed compound (called Metabolite B). The example compound structures and the structures of the two metabolites for the respective compounds were as shown in Table 4. The Metabolites B formed under the conditions tested were compounds with structure as Examples 17 and 18:

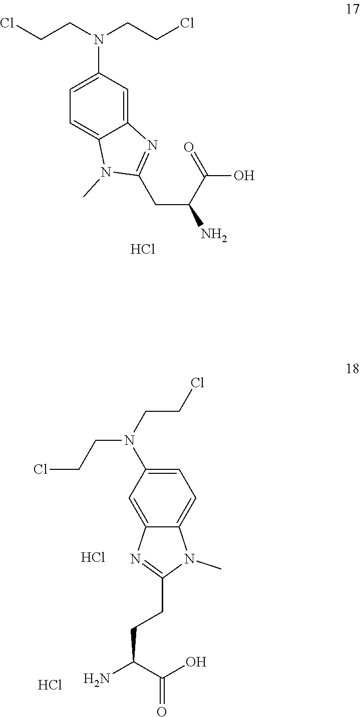 [SMILES: Cn1c(C[C@H](N)C(=O)O)nc2cc(N(CCCl)CCCl)ccc21 ; Cl ; Cl ; Cn1c(CC[C@H](N)C(=O)O)nc2cc(N(CCCl)CCCl)ccc21 ; Cl]

TABLE 4

| Ex | Structure | Metabolite A | Metabolite B |
|---|---|---|---|
| 1 | 1 | 20 | 18 |
| | 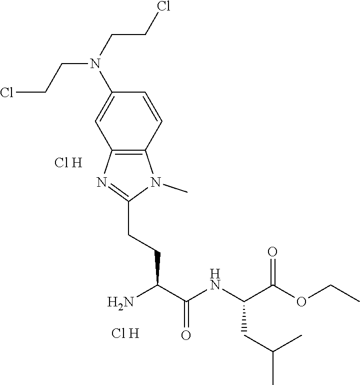 | 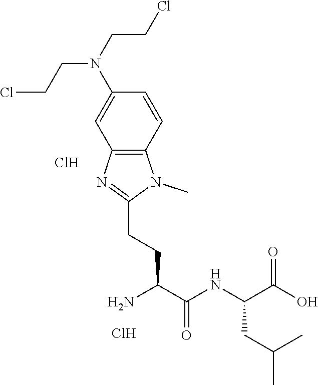 [SMILES: CC(C)C[C@H](NC(=O)[C@@H](N)CCc1nc2cc(N(CCCl)CCCl)ccc2n1C)C(=O)O ; Cl ; Cl] | |
| 2 | 2 | 39 | 18 |
| | 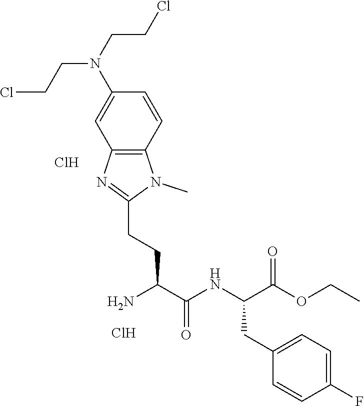 [SMILES: CCOC(=O)[C@H](Cc1ccc(F)cc1)NC(=O)[C@@H](N)CCc1nc2cc(N(CCCl)CCCl)ccc2n1C ; Cl ; Cl] | 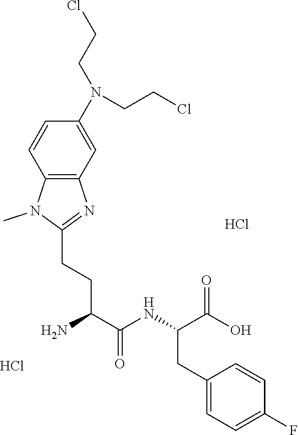 [SMILES: Cn1c(CC[C@H](N)C(=O)N[C@@H](Cc2ccc(F)cc2)C(=O)O)nc2cc(N(CCCl)CCCl)ccc21 ; Cl ; Cl] | |
| 3 | 3 | 19 | 18 |
| | 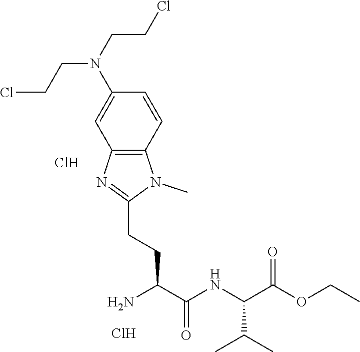 [SMILES: CCOC(=O)[C@@H](NC(=O)[C@@H](N)CCc1nc2cc(N(CCCl)CCCl)ccc2n1C)C(C)C ; Cl ; Cl] | 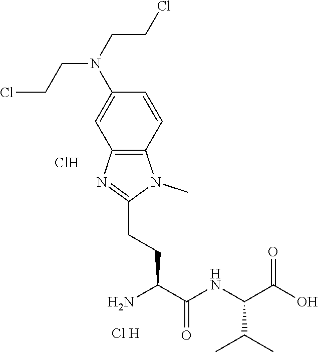 | |
| 4 | 4 | 44 | 17 |
| | 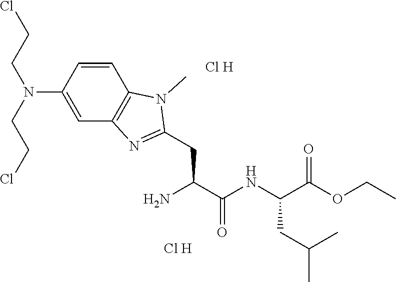 | 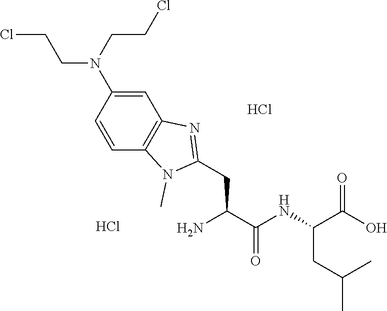 [SMILES: CC(C)C[C@H](NC(=O)[C@@H](N)Cc1nc2cc(N(CCCl)CCCl)ccc2n1C)C(=O)O ; Cl ; Cl] | |
| 6 | 6 | 42 | 17 |
| | 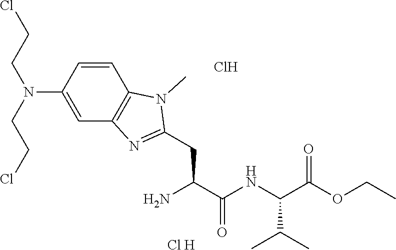 [SMILES: CCOC(=O)[C@@H](NC(=O)[C@@H](N)Cc1nc2cc(N(CCCl)CCCl)ccc2n1C)C(C)C ; "Cl H" ; Cl] | 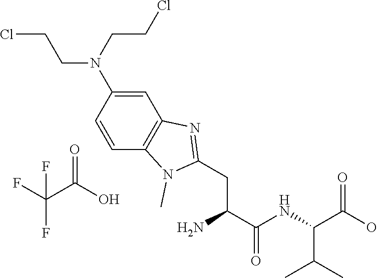 | |
| 7 | 7 | 37 | 18 |
| | 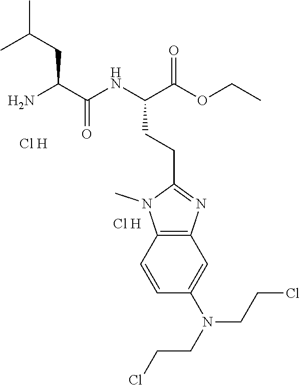 | 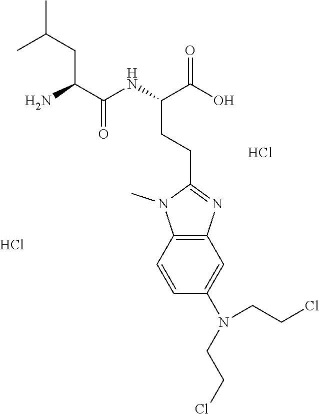 [SMILES: Cl ; CC(C)C[C@H](N)C(=O)N[C@@H](CCc1nc2cc(N(CCCl)CCCl)ccc2n1C)C(=O)O ; Cl] | |
| 10 | 10 | 43 | 17 |
| | 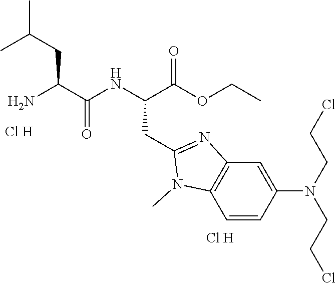 | 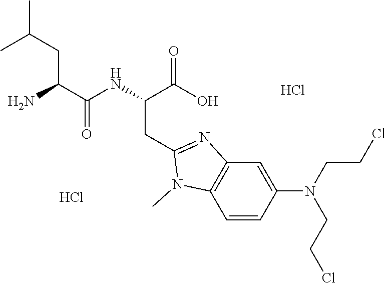 [SMILES: CC(C)C[C@H](N)C(=O)N[C@@H](Cc1nc2cc(N(CCCl)CCCl)ccc2n1C)C(=O)O ; Cl ; Cl] | |

The intracellular concentration of the metabolites slowly decreased over time. Little or undetectable extracellular concentrations of the metabolites can be detected in the media outside cells. These results indicate that hydrolysis of the Example compounds took place inside the cells and that the metabolites of the Example compounds were retained in the cells.

### Biological Example 6—Assessment of Direct DNA Damage by Test Compounds

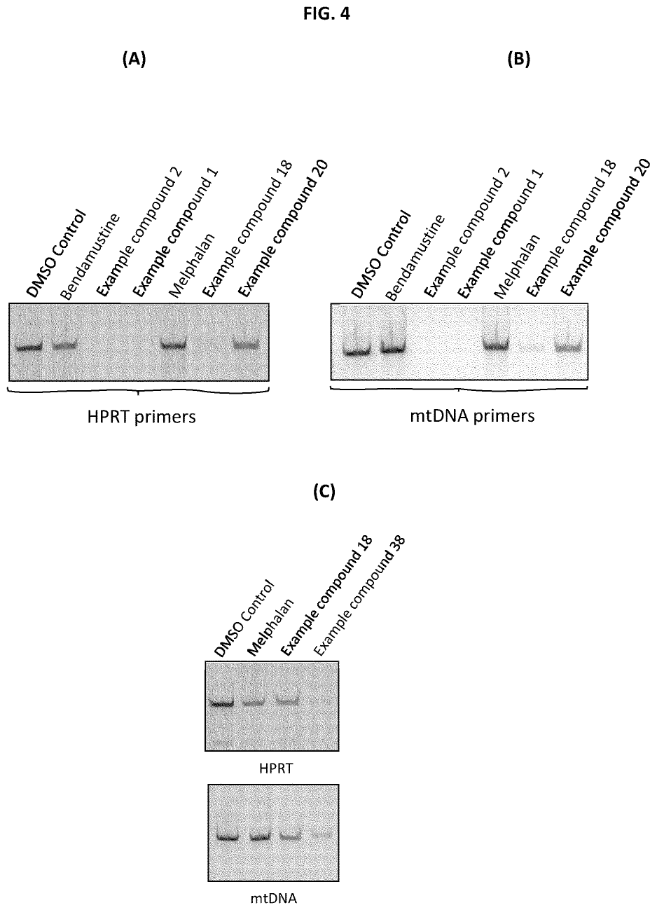

Method: DNA was prepared from MM.1S cells by QIAGEN genomic tip 20/G according to protocol described in Furda et al., Methods Mol Biol, 2012. 1 μg of DNA was treated with 0.25 μM compound for 30 minutes at 37° C. DMSO was used as control treatment. 20 ng of the treated DNA was PCR amplified using Phusion Hot Start II High Fidelity PCR Master Mix (Thermo Scientific) and primers directed to either HPRT or primers directed to mitochondrial DNA (mtDNA) was used, generating 10.4 or 8.9 kb fragments, respectively. The PCR reactions were separated on a 1% agarose gel and visualized by ChemiDoc™ MP Imaging System (BioRad). Example compounds 1, 2 and 38 were investigated, as were compounds 18 and 20, which are metabolites of compound 1. They were compared with bendamustine, melphalan and DMSO control. The results for compounds 1, 2, 18 and 20 with the HPRT primers are shown in FIG. 4(A); the results with the mtDNA primers are shown in FIG. 4(B). The results for compounds 18 and 38 with the HPRT primers and the mtDNA primers are shown in FIG. 4(C).

Results: It is seen in FIG. 4 that Melphalan and Bendamustine had no or very minor effect on DNA damage on both nuclear and mitochondrial DNA at the concentration tested. Example compounds 1 and 2 had a strong DNA damaging effect on both nuclear and mitochondrial DNA, as shown by the fact that no PCR bands could be detected on the gel. Example compounds 18 and 38 effectively damaged both nuclear and mitochondrial DNA and only a weak PCR band could be detected. Example compound 20 also had a DNA damaging effect but to a lesser degree compared to Example compounds 1, 2 and 18, but greater than Bendamustine or Melphalan.

### Biological Example 7—Assessment of DNA Fragmentation Caused by Test Compounds

Method: 1×10⁶ MM.1S cells (CRL-2974, ATCC) were treated with Example compound 1 or Bendamustine at 0.006, 0.06, 0.6 or 6 μM for 24 h. After incubation, the cells were washed with PBS (10010-015, Gibco), fixed in Flow Cytometry Fixation Buffer (FC004, R&D Systems) for 10 min and stored in 70% ethanol at −20° C. overnight. The deoxythymidine analog (BrdUTP) was present in the experiment and served to label the DNA break sites. Next day, the cells were washed and stained using an APO-BrdU™ TUNEL Assay Kit, according to the manufacturer's protocol (A23210, Invitrogen), followed by the analysis using BD FACSCanto™ II. The percentage ofTUNEL positive cells indicates the percentage of cells with fragmented DNA.

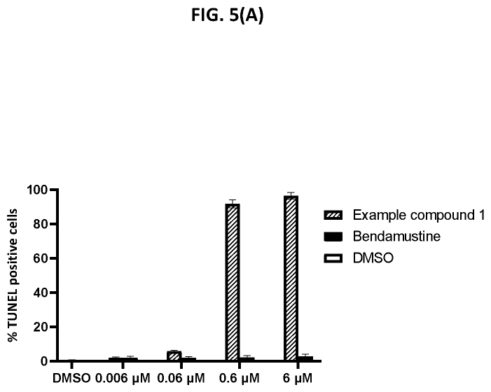

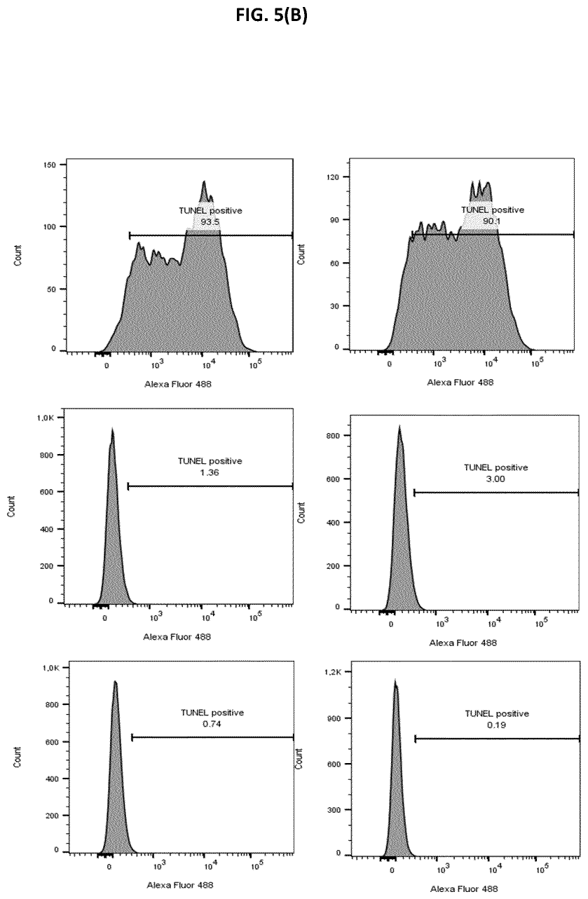

Results: The dose response effect of Example compound 1 and Bendamustine on DNA damage at 24-h incubation is shown in FIG. 5(A). All data were expressed as mean with SD (n=2). 24-h exposure to Example compound 1 induced DNA damage in a dose-dependent manner, while no obvious effect of Bendamustine on DNA damage was found upon 24-h incubation. Representative FACS histograms of Alexa fluor 488 intensity after 24-h treatment of 0.6 μM test compound or Bendamustine (n=2) are shown in FIG. 5(B). The top pair of FACS histograms are for Example compound 1; the middle pair are for Bendamustine and the bottom pair are for the DMSO control. Noticeably, the treatment with 0.6 μM Example compound 1 for 24 h caused DNA breaks in >90% of tested cells, but that was not the case for Bendamustine. Example compound 1 is more potent in causing DNA damage.

### PREPARATION EXAMPLES

### Example Preparation 1

Example compound 1*: 20 mg (free base)
Betadex Sulfobutyl Ether Sodium: 200 mg
Water for injection 10 mL
*Ethyl (2S)-2-[[(2S)-2-amino-4-[5-[bis(2-chloroethyl)amino]-1-methyl-benzimidazol-2-yl]butanoyl]amino]-4-methyl-pentanoate

### Example Preparation 2 (pH 4.5)

Example compound 1*: 20 mg (free base)
Betadex Sulfobutyl Ether Sodium: 200 mg
Citric acid: 11.0 mg
Tri sodium citrate dihydrate: 17.2 mg
Water for injection 10 mL

### Example Preparation 3 (pH 4.9)

Example compound 1*: 20 mg (free base)
Betadex Sulfobutyl Ether Sodium: 200 mg
Citric acid: 8.2 mg
Tri sodium citrate dihydrate: 21.8 mg
Water for injection 10 mL

### Stability of Example Compound 1 with Various Excipients

Ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1; 19.2 mg, 16.7 mg free base) was dissolved in dimethylacetamide (419 μL) to give a 40 mg/mL stock solution.

Solutions of 0.4 mg/mL of Example compound 1 were prepared by diluting the stock solution in dimethylacetamide (40 mg/mL, 10 μL) with different solutions (990 μL) according to the Table 5 and the stability after 4 h at room temperature was analyzed by HPLC (Analytical HPLC was carried out on an Agilent Series 1100 system using a Kinetex XB C18 (2.6 μm, 3.0×50 mm) column with 0.1% TFA in H₂O/CH₃CN as mobile phase (acid conditions) (flow 1 mL/min). The excipients were reported in weight ratio excipient:Example compound 1 (free base).

TABLE 5

| | | | Ex 1 remaining |
|---|---|---|---|
| | | Weight | after 4 h |
| | | ratio of | at room |
| Excipient | Solvent | excipient:Ex 1 | temp (%) |
| None | Dimethylacetamide | 0 | 103 |
| None | Ultrapure | 0 | 85 |
| | water (MilliQ) | | |
| None | 0.9% Saline | 0 | 93 |
| None | 5% Glucose | 0 | 80 |
| None | DPBS¹ | 0 | 10 |
| 2-Hydroxypropyl-β- | Ultrapure | 5 | 78 |
| cyclodextrin (Kleptose) | water (MilliQ) | | |
| 2-Hydroxypropyl-β- | Ultrapure | 10 | 84 |
| cyclodextrin (Kleptose) | water (MilliQ) | | |
| 2-Hydroxypropyl-β- | Ultrapure | 20 | 85 |
| cyclodextrin (Kleptose) | water (MilliQ) | | |
| Betadex Sulfobutyl Ether | Ultrapure | 5 | 99 |
| Sodium (Dexolve) | water (MilliQ) | | |
| Betadex Sulfobutyl Ether | Ultrapure | 10 | 101 |
| Sodium (Dexolve) | water (MilliQ) | | |
| Betadex Sulfobutyl Ether | Ultrapure | 20 | 98 |
| Sodium (Dexolve) | water (MilliQ) | | |
| γ-Cyclodextrin | Ultrapure | 5 | 78 |
| | water (MilliQ) | | |
| γ-Cyclodextrin | Ultrapure | 10 | 82 |
| | water (MilliQ) | | |
| γ-Cyclodextrin | Ultrapure | 20 | 86 |
| | water (MilliQ) | | |
| 2-Hydroxypropyl-γ- | Ultrapure | 5 | 82 |
| cyclodextrin | water (MilliQ) | | |
| 2-Hydroxypropyl-γ- | Ultrapure | 10 | 86 |
| cyclodextrin | water (MilliQ) | | |
| 2-Hydroxypropyl-γ- | Ultrapure | 20 | 79 |
| cyclodextrin | water (MilliQ) | | |

¹Dulbecco's phosphate-buffered saline: poor solubility.

The experiment shows that in ultrapure water the best excipient was Betadex Sulfobutyl Ether Sodium, where very little material had degraded after four hours at room temperature.

1. A pharmaceutical preparation comprising a negatively charged cyclodextrin or a pharmaceutically acceptable derivative of a negatively charged cyclodextrin, and a compound according to formula (I), or a pharmaceutically acceptable salt, ester, amide or carbamate thereof, or a salt of such an ester, amide or carbamate 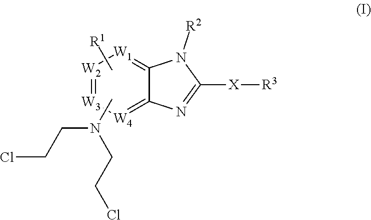
wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is a group according to formula (II): 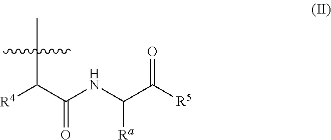
or formula (III): 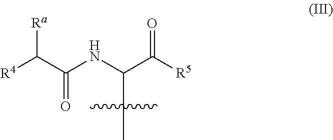
wherein
R⁴ is selected from the group consisting of N(R^{c})(R^{d}) and formula (IV): 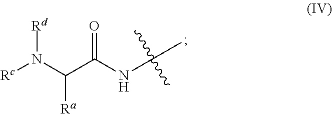
when R⁴ is formula (IV), R⁵ is R^{b}; and when R⁴ is N(R^{c})(R^{d}), R⁵ is selected from the group consisting of R^{b} and formula (V): 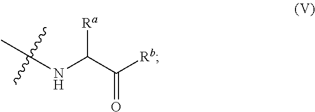
wherein each R^{a} is independently selected from the group consisting of H; C₁₋₆alkyl; —CH₂-phenyl; or —CH₂-3 to 12-membered heterocyclyl comprising 1, 2, 3 or 4 heteroatoms selected from N, O and S; wherein said C₁₋₆alkyl is optionally substituted by 1, 2 or 3 substituents independently selected from the group consisting of —OH, —OC₁₋₆alkyl, —NH₂, —NHC(═NH)NH₂, —C(O)OH, —C(O)NH₂, —SH, —SCH₃, and halogen; and said phenyl or heterocyclyl is optionally substituted by 1, 2 or 3 substituents independently selected from the group consisting of halogen, —NH₂, —OH, —OC₁₋₆alkyl and —NO₂;
R^{b} is selected from the group consisting of —OH; —N(R^{e})(R^{f}); and —OC₁₋₆alkyl optionally substituted by one or more groups selected from halogen, —OH, —CN, —N(R^{e})(R^{f}), —C₆₋₁₀aryl, or 3 to 12 membered heterocycle comprising one or more O, N or S atoms and optionally substituted by 1, 2 or 3 halogens, and/or wherein said alkyl is optionally interrupted by 1, 2 or 3 O, N or S atoms;
R^{c} and R^{d} are each independently selected from the group consisting of H; —C₁₋₆alkyl, C(O)C₁₋₆alkyl; and —CH₂-phenyl, wherein said alkyl or said phenyl is optionally substituted by 1, 2 or 3 groups selected from halogen; and
R^{e} and R^{f} are each independently selected from the group consisting of H and —C₁₋₆alkyl, wherein said alkyl is optionally substituted by 1, 2 or 3 groups selected from halogen; or R^{e} and R^{f} together with the nitrogen atom to which they are attached form a 4-, 5- or 6-membered heterocycle which is optionally substituted by 1, 2 or 3 groups selected from halogen.

2. The pharmaceutical preparation of claim 1, wherein the compound of formula (I) or the pharmaceutically acceptable salt, ester, amide or carbamate thereof, or a salt of such an ester, amide or carbamate has the structure (Ia): 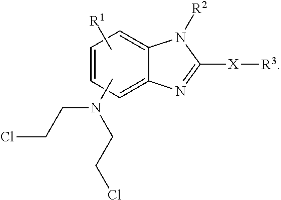
or structure (Ib):

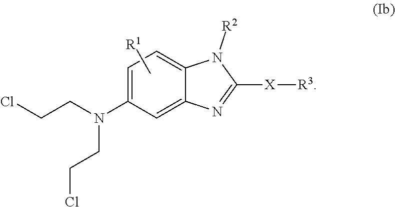

3. The pharmaceutical preparation of claim 1, wherein R¹ is H.

4. The pharmaceutical preparation of claim 1, wherein R² is selected from the group consisting of phenyl optionally substituted by 1, 2 or 3 halogens; and —C₁₋₆alkyl optionally substituted by 1, 2 or 3 halogens; for example —C₁₋₄alkyl optionally substituted by 1, 2 or 3 halogens, for example methyl.

5. The pharmaceutical preparation of claim 1, wherein X is —CH₂— or —CH₂—CH₂—.

6. The pharmaceutical preparation of claim 1, wherein R³ is a group according to formula (IIa): 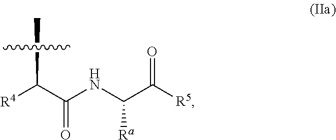
or formula (IIIa):

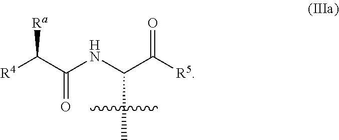

7. The pharmaceutical preparation of claim 1, wherein R⁴ is selected from the group consisting of N(R^{c})(R^{d}) and formula (IVa):

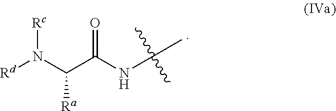

8. The pharmaceutical preparation of claim 1 wherein, when R⁴ is formula (IV) or (IVa), R⁵ is R^{b}; and when R⁴ is N(R^{c})(R^{d}), R⁵ is selected from the group consisting of R^{b} and formula (Va), wherein formula (Va) has the following structure,

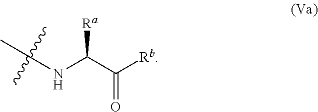

9. The pharmaceutical preparation of claim 1, wherein R⁴ is NH₂.

10. The pharmaceutical preparation of claim 1 wherein each R^{a} is independently selected from the group consisting of C₁₋₆alkyl and —CH₂-phenyl; wherein said C₁₋₆alkyl is optionally substituted by —OH; —NH₂; —NHC(═NH)NH₂; —C(O)OH; —C(O)NH₂; —SH; —SCH₃; or halogen (for example, F or Cl); and said phenyl is optionally substituted by 1, 2 or 3 substituents independently selected from the group consisting of halogen (for example, F or Cl); —NH₂; —OH; —O—C₁₋₆alkyl; and —NO₂.

11. The pharmaceutical preparation of claim 10, wherein each R^{a} is selected from the group consisting of —C₁₋₆alkyl and —CH₂-phenyl; wherein said phenyl is optionally substituted by 1, 2 or 3 halogen substituents.

12. The pharmaceutical preparation of claim 1, wherein when R⁴ is formula (IV) or (IVa), R⁵ is —OC₁₋₆alkyl; and when R⁴ is NH₂, R⁵ is —OC₁₋₆alkyl or formula (V) or (Va).

13. The pharmaceutical preparation of claim 12, wherein when R^{b} is —OC₁₋₆alkyl, it is selected from the group consisting of methoxy, ethoxy and isopropoxy.

14. The pharmaceutical preparation of claim 1, wherein the compound of formula (I) is selected from the group consisting of:
Ethyl (2S)-2-[[(2S)-2-amino-4-[5-[bis(2-chloroethyl)amino]-1-methyl-benzimidazol-2-yl]butanoyl]amino]-4-methyl-pentanoate;
Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate;
Ethyl (2S)-2-[[(2S)-2-amino-4-[5-[bis(2-chloroethyl)amino]-1-methyl-benzimidazol-2-yl]butanoyl]amino]-3-methyl-butanoate;
Ethyl (2S)-2-[[(2S)-2-amino-3-[5-[bis(2-chloroethyl)amino]-1-methyl-benzimidazol-2-yl]propanoyl]amino]-4-methyl-pentanoate;
Ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate;
Ethyl (2S)-2-[[(2S)-2-amino-3-[5-[bis(2-chloroethyl)amino]-1-methyl-benzimidazol-2-yl]propanoyl]amino]-3-methyl-butanoate;
Ethyl (2S)-2-[[(2S)-2-amino-4-methyl-pentanoyl]amino]-4-[5-[bis(2-chloroethyl)amino]-1-methyl-benzimidazol-2-yl]butanoate;
Ethyl (2S)-2-[[(2S)-2-amino-3-(4-fluorophenyl)propanoyl]amino]-4-[5-[bis(2-chloroethyl)amino]-1-methyl-benzimidazol-2-yl]butanoate;
Ethyl (2S)-2-[[(2S)-2-amino-3-methyl-butanoyl]amino]-4-[5-[bis(2-chloroethyl)amino]-1-methyl-benzimidazol-2-yl]butanoate;
Ethyl (2S)-2-[[(2S)-2-amino-4-methyl-pentanoyl]amino]-3-[5-[bis(2-chloroethyl)amino]-1-methyl-benzimidazol-2-yl]propanoate;
Ethyl (2S)-2-[[(2S)-2-amino-3-(4-fluorophenyl)propanoyl]amino]-3-[5-[bis(2-chloroethyl)amino]-1-methyl-benzimidazol-2-yl]propanoate;
Ethyl (2S)-2-[[(2S)-2-amino-3-methyl-butanoyl]amino]-3-[5-[bis(2-chloroethyl)amino]-1-methyl-benzimidazol-2-yl]propanoate;
Ethyl (2S)-2-[[(2S)-2-amino-4-[5-[bis(2-chloroethyl)amino]-1-phenyl-benzimidazol-2-yl]butanoyl]amino]-4-methyl-pentanoate; Methyl (2S)-2-[[(2S)-2-amino-3-[5-[bis(2-chloroethyl)amino]-1-methyl-benzimidazol-2-yl]propanoyl]amino]-4-methyl-pentanoate;
Isopropyl (2S)-2-[[(2S)-2-amino-3-[5-[bis(2-chloroethyl)amino]-1-methyl-benzimidazol-2-yl]propanoyl]amino]-4-methyl-pentanoate; ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate;
Ethyl (2S)-2-[[(2R)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate;
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-3-(4-fluorophenyl)propanoate;
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-4-methyl-pentanoate;
2-Morpholinoethyl (2S)-2-[[(2S)-2-amino-4-[5-[bis(2-chloroethyl)amino]-1-methyl-benzimidazol-2-yl]butanoyl]amino]-4-methyl-pentanoate;
2-Isopropoxyethyl (2S)-2-[[(2S)-2-amino-4-[5-[bis(2-chloroethyl)amino]-1-methyl-benzimidazol-2-yl]butanoyl]amino]-4-methyl-pentanoate;
Isopropyl (2S)-2-[[(2S)-2-amino-4-[5-[bis(2-chloroethyl)amino]-1-methyl-benzimidazol-2-yl]butanoyl]amino]-4-methyl-pentanoate;
Methyl (2S)-2-[[(2S)-2-amino-4-[5-[bis(2-chloroethyl)amino]-1-methyl-benzimidazol-2-yl]butanoyl]amino]-4-methyl-pentanoate;
3-(Dimethylamino)propyl (2S)-2-[[(2S)-2-amino-4-[5-[bis(2-chloroethyl)amino]-1-methyl-benzimidazol-2-yl]butanoyl]amino]-4-methyl-pentanoate;
(2-Methoxy-1-methyl-ethyl) (2S)-2-[[(2S)-2-amino-4-[5-[bis(2-chloroethyl)amino]-1-methyl-benzimidazol-2-yl]butanoyl]amino]-4-methyl-pentanoate;
(2S)-2-[[(2S)-2-Amino-4-[5-[bis(2-chloroethyl)amino]-1-methyl-benzimidazol-2-yl]butanoyl]amino]-N,N,4-trimethyl-pentanamide;
(2S)-2-[[(2S)-2-amino-4-[5-[bis(2-chloroethyl)amino]-1-methyl-benzimidazol-2-yl]butanoyl]amino]-N,4-dimethyl-pentanamide;
Ethyl (2S)-2-[[(2S)-4-[5-[bis(2-chloroethyl)amino]-1-methyl-benzimidazol-2-yl]-2-(methylamino)butanoyl]amino]-4-methyl-pentanoate;
Ethyl (2S)-2-[[(2S)-2-acetamido-4-[5-[bis(2-chloroethyl)amino]-1-methyl-benzimidazol-2-yl]butanoyl]amino]-3-(4-fluorophenyl)propanoate;
Ethyl (2S)-2-[[(2S)-2-acetamido-3-[5-[bis(2-chloroethyl)amino]-1-methyl-benzimidazol-2-yl]propanoyl]amino]-4-methyl-pentanoate;
Ethyl (2S)-2-[[(2S)-2-acetamido-3-[5-[bis(2-chloroethyl)amino]-1-methyl-benzimidazol-2-yl]propanoyl]amino]-3-(4-fluorophenyl)propanoate;
Ethyl (2S)-2-amino-4-[5-[bis(2-chloroethyl)amino]-1-methyl-benzimidazol-2-yl]butanoate;
Ethyl (2R)-2-[[(2S)-2-amino-4-[5-[bis(2-chloroethyl)amino]-1-methyl-benzimidazol-2-yl]butanoyl]amino]-4-methyl-pentanoate;
Ethyl (2S)-2-[[(2R)-2-amino-4-[5-[bis(2-chloroethyl)amino]-1-methyl-benzimidazol-2-yl]butanoyl]amino]-4-methyl-pentanoate;
Ethyl (2R)-2-[[(2R)-2-amino-4-[5-[bis(2-chloroethyl)amino]-1-methyl-benzimidazol-2-yl]butanoyl]amino]-4-methyl-pentanoate; Ethyl (2S)-2-[[(2S)-2-amino-4-[6-[bis(2-chloroethyl)amino]-3-methyl-imidazo[4,5-b]pyridin-2-yl]butanoyl]amino]-4-methyl-pentanoate;
Ethyl (2S)-2-[[(2S)-2-amino-4-[5-[bis(2-chloro-1,1,2,2-tetradeuterio-ethyl)amino]-1-methyl-benzimidazol-2-yl]butanoyl]amino]-4-methyl-pentanoate;
2S)-2-[[(2S)-2-amino-4-[5-[bis(2-chloroethyl)amino]-1-methyl-benzimidazol-2-yl]butanoyl]amino]-3-methyl-butanoic acid; and
(2S)-2-[[(2S)-2-amino-4-[5-[bis(2-chloroethyl)amino]-1-methyl-benzimidazol-2-yl]butanoyl]amino]-4-methyl-pentanoic acid;
(2S)-2-[2S)-4-[5-[bis(2-chloroethyl)amino-1-methyl-benzimidazol-2-yl]-2-(methylamino)butanoyl]amino]-4-methyl-pentanoic acid;
(2S)-2-[2S)-2-Amino-4-methyl-pentanoylamino]-4-[5-[bis(2-chloroethyl)amino]-1-methyl-benzimidazol-2-yl]butanoic acid;
(2S)-2-[(2S)-2-Amino-4-[5-[bis(2-chloroethyl)amino-1-methyl-benzimidazol-2-yl]butanol]amino]~3-(4-fluorophenylpropanoic acid;
(2S)-2-[[2S)-2-Anino-4-[5-[bis(2-chloroethyl)amino]-1-phenyl-benzimidazol-2-yl]butanoyl]amino]-4-methyl-pentanoic acid:
2S)-2-[[(2S)-2-amino-3-[5-[bis(2-chloroethyl)amino]-1-methyl-benzimidazol-2-yl]propanoyl]amino]-3-methyl-butanoic acid;
(2S)-2-[[(2S)-2-amino-4-methyl-pentanoyl]amino]-3-[5-bis 2-chloroethyl)amino-1-methyl-benzimidazol-2-yl]propanoic acid:
(2S)-2-[[(2S)-2-amino-3-[5-[bis(2-chloroethyl)amino]-methyl-benzimidazol-2-yl]propanoyl]amino]-4-methyl-pentanoic acid; and
(2S)-2-[[(2S)-2-amino-4-[5-[bis(2-chloro-1,1,2,2-tetradeuterio-ethyl)amino]-1-methyl-benzimidazol-2-yl]butanoyl]amino]-4-methyl-pentanoic acid;
or a pharmaceutically acceptable salt, ester, amide or carbamate thereof, or a salt of such an ester, amide or carbamate.

15. (canceled)

16. A pharmaceutical preparation comprising a negatively charged cyclodextrin or a pharmaceutically acceptable derivative of a negatively charged cyclodextrin, and a compound according to formula (I), or a pharmaceutically acceptable salt, ester, amide or carbamate thereof, or a salt of such an ester, amide or carbamate 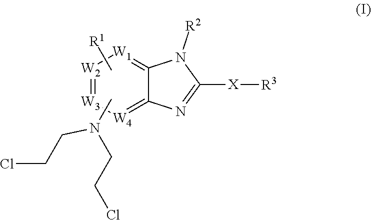
wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is formula (VIa): 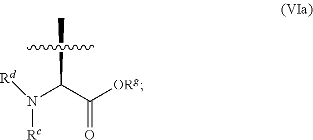
wherein, R^{c} and R^{d} are each independently selected from the group consisting of H, —C₁₋₆alkyl, C(O)C₁₋₆alkyl and —CH₂-phenyl, wherein said alkyl or said phenyl is optionally substituted by 1, 2 or 3 groups selected from halogen; and
R^{g} is selected from the group consisting of H and —C₁₋₆alkyl, wherein said alkyl is optionally substituted by 1, 2 or 3 groups selected from halogen.

17. The pharmaceutical preparation of claim 16 wherein:
(a) the compound of formula (I) or the pharmaceutically acceptable salt, ester, amide or carbamate thereof, or a salt of such an ester, amide or carbamate has the structure (Ia); 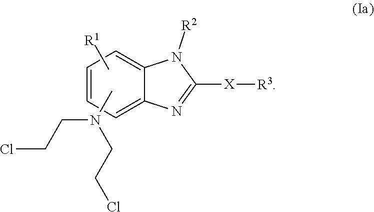
the structure (Ib): 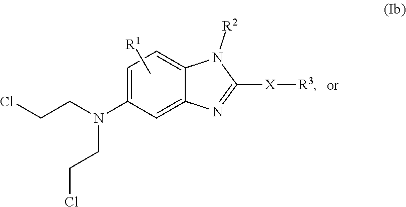
or R¹ is H, R² is selected from the group consisting of phenyl optionally substituted by 1, 2 or 3 halogens and —C1-6alkyl optionally substituted by 1, 2 or 3 halogens, or X is —CH₂— or —CH₂—CH₂—;
and
(b) R^{c} and R^{d} are each H; and R^{g} is H.

18. The pharmaceutical preparation of claim 16 wherein the compound of formula (I) is selected from the group consisting of:
(2S)-2-amino-3-[5-[bis(2-chloroethyl)amino]-1-methyl-benzimidazol-2-yl]propanoic acid;
(S)-2-amino-4-(5-(bis(2-chloroethyl)amino)-1-methyl-1H-benzo[d]imidazol-2-yl)butanoic acid;
Ethyl (2S)-2-amino-4-[5-[bis(2-chloroethyl)amino]-1-methyl-benzimidazol-2-yl]butanoate;
(2S)-2-Amino-4-[5-[bis(2-chloroethyl)amino]-1-phenyl-benzimidazol-2-yl]butanoic acid; and
(2S)-2-amino-4-[5-[bis(2-chloro-1,1,2,2-tetradeuterio-ethyl)amino]-1-methyl-benzimidazol-2-yl]butanoic acid;
or a pharmaceutically acceptable salt, amide or carbamate thereof, or a salt of such an ester, amide or carbamate.

19. (canceled)

20. The pharmaceutical preparation of claim 1 which comprises a hydrochloride salt of the compound of formula (I) or an ester, amide or carbamate thereof.

21. The pharmaceutical preparation of claim 1 comprising a pharmaceutically acceptable derivative of a negatively charged cyclodextrin.

22. The pharmaceutical preparation of claim 21, wherein the pharmaceutically acceptable derivative of a negatively charged cyclodextrin is a sulfoalkyl ether derivatized cyclodextrin or sulfobutyl ether derivatized cyclodextrin.

23. (canceled)

24. The pharmaceutical preparation of claim 22, wherein the average number of sulfoalkyl groups per cyclodextrin ring, expressed as average degree of substitution is from 5 to 8.

25. The pharmaceutical preparation of claim 1, wherein the negatively charged cyclodextrin or pharmaceutically acceptable derivative of a negatively charged cyclodextrin is a negatively charged β-cyclodextrin or a pharmaceutically acceptable derivative of a negatively charged β-cyclodextrin.

26. The pharmaceutical preparation of claim 1, wherein the negatively charged cyclodextrin or pharmaceutically acceptable derivative of a negatively charged cyclodextrin is Betadex Sulfobutyl Ether Sodium.

27. The pharmaceutical preparation of claim 1, comprising citric acid and/or one or more salts of citric acid (such as trisodium citrate dihydrate).

28. The pharmaceutical preparation of claim 1, wherein the weight ratio (w/w) between the negatively charged cyclodextrin or the pharmaceutically acceptable derivative of a negatively charged cyclodextrin and the compound of formula (I) or the pharmaceutically acceptable salt, ester, amide or carbamate thereof, or a salt of such an ester, amide or carbamate, excluding the mass of any counterion, is from 1:1 to 100:1, from 2:1 to 50:1, or from 5:1 to 20:1.

29. The pharmaceutical preparation of claim 1, wherein said pharmaceutical preparation is a lyophilized powder.

30. The pharmaceutical preparation of claim 29, wherein the pharmaceutical preparation comprises 1 mg to 150 mg of the compound of formula (I) or the pharmaceutically acceptable salt, ester, amide or carbamate thereof, or a salt of such an ester, amide or carbamate, excluding the mass of any counterion.

31. The pharmaceutical preparation of claim 29, which is substantially free from organic solvent.

32. The pharmaceutical preparation of claim 1, comprising one or more additional therapeutic agent(s) selected from the group consisting of steroids, immunomodulatory drugs (IMiDs), proteasome inhibitors (PIs), histone deacetylase (HDAC) inhibitors, conventional chemotherapy, checkpoint inhibitors, nuclear transport inhibitors, anti-apoptotic inhibitors, adoptive cell therapy, bi-specific T-cell engagers (BiTEs), B-cell targeting agents, and monoclonal antibodies.

33. (canceled)

34. A composition comprising the pharmaceutical preparation of claim 1 and a physiologically acceptable aqueous solvent.

35. The composition of claim 34, wherein the physiologically acceptable aqueous solvent is a glucose solution, a saline solution, or a mixture thereof.

36. A kit comprising the pharmaceutical preparation of claim 1 and a physiologically acceptable aqueous solvent or diluent.

37-43. (canceled)

44. A method for the treatment or prophylaxis of cancer, comprising administering an effective amount of a pharmaceutical preparation of claim 1 to a subject in need thereof, for example wherein said cancer is selected from the group consisting of hematologic/blood cell cancers such as a leukemia (for example, acute lymphoblastic leukemia including adult and childhood acute lymphoblastic leukemia; acute myeloid leukemia including adult and childhood acute myeloid leukemia; chronic lymphocytic leukemia such as B Cell chronic lymphocytic leukemia; chronic myelogenous leukemia; and hairy cell leukemia); lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; primary mediastinal large B cell lymphoma; mantle cell lymphoma; diffuse large B cell lymphoma; and primary central nervous system lymphoma); and other hematologic cancers (for example, chronic myeloproliferative disorders; multiple myeloma/plasma cell neoplasm; myelodysplastic syndromes; and myelodysplastic/myeloproliferative disorders); osteosarcoma, ovarian cancer, breast cancer; lung cancer; glioblastoma; retinoblastoma and metastases of the aforementioned cancers.

45. (canceled)

46. The method of claim 44, wherein the pharmaceutical preparation is administered with one or more additional therapeutic agents selected from the group consisting of a steroid, an IMiD, a PI, a HDAC inhibitor, conventional chemotherapy, a checkpoint inhibitor, a nuclear transport inhibitor, an anti-apoptotic inhibitor, an adoptive cell therapy, a BiTE, a B-cell targeting agents, and a monoclonal antibody.
